(19) United States
(12) Patent Application Publication
(10) Pub. No.: US 2004/0241717 A1
(43) Pub. Date: Dec. 2, 2004

(54) Oligomeric compounds for the modulation of thioredoxin expression

(75) Inventors: Bo Hansen, Copenhagen K (DK); Charlotte Albaek Thrue, Copenhagen K (DK); Majken Westergaard, Birkered (DK); Kamille Dumong Petersen, Lejre (DK); Margit Wissenbach, Fredensborg (DK)

Correspondence Address: Peter F. Corless, EDWARDS & ANGELL, LLP, P. O. Box 55874, Boston, MA, 02205, US

(73) Assignee: Santaris Pharma A/S

(21) Appl. No.: 10/776,933

(22) Filed: Feb. 10, 2004

Related U.S. Application Data
(60) Provisional application No. 60/446,374, filed on Feb. 10, 2003.

(51) Int. Cl. C12Q 1/68; C07H 21/02

(52) U.S. Cl. 435/6; 536/23.1; 544/244

(57) ABSTRACT

Oligonucleotides directed against the TRX gene are provided for modulating the expression of TRX. The compositions comprise oligonucleotides, particularly antisense oligonucleotides, targeted to nucleic acids encoding the TRX. Methods of using these compounds for modulation of TRX expression and for the treatment of diseases associated with either overexpression of TRX, expression of mutated TRX or both are provided. Examples of diseases are cancer such as lung, breast, colon, prostate, pancreas, lung, liver, thyroid, kidney, brain, testes, stomach, intestine, bowel, spinal cord, sinuses, bladder, urinary tract or ovaries cancers. The oligonucleotides may be composed of deoxyribonucleosides or a nucleic acid analogue such as for example locked nucleic acid or a combination thereof.

### FIELD OF THE INVENTION

The present invention provides compositions and methods for modulating the expression of TRX. In particular, this invention relates to oligomeric compounds and preferred such compounds are oligonucleotides, which are specifically hybridisable with nucleic acids encoding TRX. The oligonucleotide compounds have been shown to modulate the expression of TRX and pharmaceutical preparations thereof and their use as treatment of cancer diseases are disclosed.

### BACKGROUND OF THE INVENTION

This invention relates to oligonucleotides (e.g. containing LNA) that are complementary to the human thioredoxin (TRX) putative oncogene, which has been found to modulate tumor cell growth and apoptosis inhibition in a variety of human cancers. TRX has also been closely linked with drug resistance in cancer treatments (Yokomizo et al. 1995. Cancer Res. 55:4293-4296; Kahlos et al. 2001.Int.J.Cancer 20;95:198-204). T. C. Laurent first described TRX in 1964 from Escherichia Coli. It is a ubiquitous and relatively conserved approximately 12 kDa oxireductant enzyme found in both prokaryotes and eukaryots (Holmgren. 1989. J.Biol.Chem. 264:13963-13966). TRX contains a dithiol disulfide active site which is involved in redox reactions through the formation of reversible disulfide bonds and which undergoes reversible thiol reduction by the NADPH-dependant enzyme thioredoxin reductase. The active site is highly conserved and contains a Cys-Gly-Pro-Cys sequence (Holmgren 1985. Annu.Rev.Biochem. 54:237-71.:237-271). Mammalian thioredoxin family comprises TRX-1 and TRX-2. The first is the cytosolic and nuclear form and the later is the mitochondrial form. TRX-1 is the most extensively described and is a 104 amino acid protein that has been suggested to be represented in several mutated forms in the cell (Powis, et al.. 2001.Annu.Rev.Biophys.Biomol.Struct. 30:421-55.:421-455). Human TRX/TRX-1 (11.5-kDa) which is also known as Adult T-cell Leukaemia-derived Factor (ADF) (Gasdaska et al. 1994. Biochim.Biophys.Acta 1218:292-296) or Eosinophil cytotoxicity stimulating factor (Silberstein, et al. 1993. J.Biol.Chem. 268:9138-9142) has 5 cysteine residues which is 3 more than found in bacteria. These extra cysteines are responsible for the unique properties of human TRX (Gasdaska et al. 1994. Biochim.Biophys.Acta 1218:292-296). It has been shown that TRX modulates the DNA binding of transcription factors by redox control and hereby regulate gene transcription. Transcription factors described to be under TRX control are NF-κB (Matthews, et al. 1992. Nucleic Acids Res. 20:3821-3830), TFIIIC (Cromlish et al. J.Biol.Chem. 264:18100-18109), BZLF1(Bannister et al. 1991. Oncogene 6:1243-1250), p53 (Ueno, et al 1999. J.Biol.Chem. 274:35809-35815), the glucocorticoid receptor (Grippo, et al. 1983. J.Biol.Chem. 258:13658-13664) and indirectly AP-1 (Fos/Jun heterodimer)) (Abate et al. 1990. Science 249:1157-1161). TRX also increases DNA binding of AP-2, the estrogen receptor and PEBP2/CBF (Powis, et al. 2001.Annu.Rev.Biophys.Biomol.Struct. 30:421-55.:421-455). Hypoxia-inducible factor 1 alpha (HIF-1α) has been shown to increase upon TRX elevation (Welsh et al. 2002. Cancer Res. 62:5089-5095), which could potentiate TRX as a anti-tumor-angiogenisis target. Furthermore it is involved in catalysing the conversion of nucleotides to deoxynucleotides, the first step in DNA synthesis that is essential for proliferation. TRX can serve as a signal for cancer cell growth probably by enhancing the autocrine activity of growth factors (Gasdaska et al. 1995. Cell Growth Differ. 6:1643-1650). It has been suggested that TRX up-regulates the alpha subunit of the high affinity IL-2 receptor in HTLV-1 transformed T-cells (Schenk et al. 1996. J.Immunol. 156:765-771) where IL-2 might be enhanced up to 1000 fold (Powis, et al. 2001.Annu.Rev.Biophys.Biomol.Struct. 30:421-55.:421-455). TRX also increases cytokines like IL-1, IL-6, IL-8 and TNF-α (Schenk et al. 1996. J.Immunol. 156:765-771), thus influencing on immunologic disorders e.g. human rheumatoid arthritis. Stresses (e.g. hypoxia, lipopolysaccharide, O₂, hydrogen peroxide, phorbol ester, viral infection and infectious agents, X-ray radiation and UV irradiation, hormones and chemicals) induce TRX (Powis, et al. 2001.Annu.Rev.Biophys.Biomol.Struct. 30:421-55.:421-455). The promoter region of the gene encoding TRX contains a series of stress responsive elements (Taniguchi et al. 1996. Nucleic Acids Res. 24:2746-2752). TRX-1 has been found over-expressed in a number of human primary tumors, and cancer cells secrete TRX-1 by a leaderless secretory pathway through an ER-Golgi independent manner (Rubartelli et al. 1992. J.Biol.Chem. 267:24161-24164). Human TRX has been suggested to be a potential target for anti-apoptosis and anti-proliferative treatment in various cancers as well as it may play a role in a variety of human disorders (Powis, et al. 2001.Annu.Rev.Biophys.Biomol.Struct. 30:421-55.:421-455). Apoptosis has been inhibited through over-expression of TRX both in vitro and in vivo (Baker et al. 1997. Cancer Res. 57:5162-5167). Recombinant human TRX stimulates proliferation of normal cells and cultured cancer cells from a variety of solid tumors (Gasdaska et al. 1995. Cell Growth Differ. 6:1643-1650.; Oblong et al. J.Biol.Chem. 269:11714-11720) and TRX mRNA has been found to be over-expressed in human tumor cells. Redox inactive TRX on the other hand does not stimulate cell proliferation (Oblong et al. J.Biol.Chem. 269:11714-11720). Surprisingly it has been found that malignancies of certain human primary tumor cells either express or over-express TRX compared to normal tissue. Examples are found within Gastric carcinoma (Grogan et al. 2000. Hum.Pathol. 31:475-481), malignant pleural mesothelioma (Kahlos et al. 2001.Int.J.Cancer 20;95:198-204), non-small cell lung carcinoma (Soini, et al. Clin.Cancer Res. 7:1750-1757), carcinoma of liver (Nakamura et al. Cancer 69:2091-2097), uterine cervix (Fujii et al.Cancer 68: 1583-1591), pancreas cancer (Nakamura et al. Cancer Detect.Prev. 24:53-60), Colon cancer, Non-Hodgkin's lymphoma, Acute lymphocytic leukaemia and myeloma (Powis, et al. 2001.Annu.Rev.Biophys.Biomol.Struct. 30:421-55.:421-455).

The growth-stimulating and anti-apoptotic effects of TRX-1 caused by a number of anticancer drugs (for review see Powis, et al. 2001.Annu.Rev.Biophys.Biomol.Struct. 30:421-55.:421-455.) added to the findings that TRX is over-expressed and involved in a number of primary tumors makes modulation of TRX with TRX specific drugs an attractive target for drug development. Phosphorothioate antisense oligo nucleotides have been shown to specifically modulate TRX mRNA and protein (Saitoh et al. EMBO J. 17:2596-2606). (WO9938963). These phosphorthioates were all 20 or 23 bp in length (with one exception being a 17-mer).

Most of the oligonucleotides currently in clinical trials are based on the phosphorothioate chemistry from 1988, which was the first useful antisense chemistry to be developed. However, as it has become clear in recent years this chemistry has serious shortcomings that limit its clinical use. These include low affinity for their target mRNA, which negatively affects potency and puts restrictions on how small active oligonucleotides can be thus complicating manufacture and increasing treatment costs. Also, their low affinity translate into poor accessibility to the target mRNA thus complicating identification of active compounds. Finally, phosphorothioate oligonucleotides suffer from a range of side effects that narrow their therapeutic window.

To deal with these and other problems much effort has been invested in creating novel analogues with improved properties. As depicted in the scheme 1 below, these include wholly artificial analogues such as PNA and Morpholino and more conventional DNA analogues such as boranosphosphates, N3′-P5′phosphoroamidates and several 2′ modified analogues, such as 2′-F, 2′-O-Me, 2′-O-methoxyethyl (MOE) and 2′-O-(3-aminopropyl)(AP). More recently hexitol nucleic acid (HNA), 2′-F-arabino nucleic acid (2′-F-ANA) and D-cyclohexenyl nucleoside (CeNA) have been introduced.

Many of these analogues exhibit improved binding to complementary nucleic acids, improvements in bio-stability or they retain the ability to recruit a cellular enzyme, RNAseH, which is involved in the mode-of-action of many antisense compounds. None of them, however, combine all of these advantages and in many cases improvements in one of the properties compromise one or more of the other properties. Also, in many cases new complications have been noted which seriously limits the commercial value of some of the analogues. These include low solubility, complex oligomerisation chemistries, very low cellular up-take, incompatibility with other chemistries, etc. The MOE chemistry has several limitations. It has only modest affinity, which only manifests when several MOE's are inserted en block into the oligo. MOE belongs to the family of 2′-modifications and it is well known, for this group of compound, that the antisense activity is directly correlated with RNA binding affinity in vitro. A MOE 20 bp gapmer (5MOE/PO-10PS-5MOE/PO) targeting c-raf has been reported to have an IC₅₀ of about 20 nm in T24 cells and an MOE gapmer targeting PKC-a has been reported to have an IC₅₀ of 25 nm in A549 cells. In comparison, phosphorthioate compounds used in antisense experiments typically exhibit IC₅₀ in the 150 nm range. (Stein, Kreig, Applied Antisense Oligonucleotide Technology, Wiley-Liss, 1988, p 87-90)

It is a principal object of the present invention to provide novel oligomeric compounds, against the survivin mRNA. The compounds of the invention have been found to exhibit an decreased IC₅₀ (thus increased activity), thereby facilitating an effective treatment of a variety of cancer diseases in which the expression of survivin is implied as a causative or related agent. As explained in the following, this objective is best achieved through the utilisation of a super high affinity chemistry termed LNA (Locked Nucleic Acid).

The present invention is directed to oligomeric compounds, particularly LNA antisense oligonucleotides, which are targeted to a nucleic acid encoding survivin and which modulate the expression of the survivin. This modulation was particularly a very potent down regulation survivin mRNA as well as elicitation of apoptotic response. The LNA-containing oligomeric compounds can be as low as an 8-mer and certainly highly active as a 16-mers, which is considerably shorter than the reported antisense compounds targeting survivin. These 16-mer oligomeric compounds have an IC₅₀ in the sub-nanomolar range. The invention enables a considerable shortening of the usual length of an antisense oligomers (from 20-25 mers to, e.g., 8-16 mers) without compromising the affinity required for pharmacological activity. As the intrinsic specificity of an oligo is inversely correlated to its length, such a shortening will significantly increase the specificity of the antisense compound towards its RNA target. Furthermore, it is anticipated that shorter oligomeric compounds have a higher biostability and cell permeability than longer oligomeric compounds. For at least these reasons, the present invention is a considerable contribution to the art.

### SUMMARY OF THE INVENTION

The present invention is directed to oligomeric compounds, particularly LNA antisense oligonucleotides, which are targeted to a nucleic acid encoding TRX and which modulate the expression of the TRX. Pharmaceutical and other compositions comprising the oligomeric compounds of the invention are also provided.

A central aspect of the invention to provide a compound consisting of a total of 8-50 nucleotides and/or nucleotidee analogues, wherein said compound comprises a subsequence of at least 8 nucleotides or nucleotide analogues, said subsequence being located within a sequence selected from the group consisting of SEQ ID NO: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56 or 57.

Further provided are methods of modulating the expression of TRX in cells or tissues comprising contacting said cells or tissues with one or more of the oligomeric compounds or compositions of the invention. Also disclosed are methods of treating an animal or a human, suspected of having or being prone to a disease or condition, associated with expression of TRX by administering a therapeutically or prophylactically effective amount of one or more of the oligomeric compounds or compositions of the invention. Further, methods of using oligomeric compounds for the inhibition of expression of TRX and for treatment of diseases associated with TRX activity are provided. Examples of such diseases are different types of cancer, such as for instance lung, breast, colon, prostate, pancreas, lung, liver, thyroid, kidney, brain, testes, stomach, intestine, bowel, spinal cord, sinuses, bladder, urinary tract or ovaries.

### BRIEF DESCRIPTION OF THE FIGURES

FIG. 1. Illustration of the different designs of the invention: Gapmers, Head- and Tailmers and Mixmers of different composition. For the mixmer, the numbers designate the alternate contiguous stretch of DNA, β-D-oxy-LNA or α-L-LNA. In the drawing, the line is DNA, the gray shadow corresponds to α-L-LNA residues and the rectangle is β-D-oxy-LNA. TRX Northern blot of total RNA from 15PC3 that have been

FIG. 2 shows TRX Northern Blot of total RNA from 15PC3 cells treated treated with 0.2, 1, 5, 25 nM CUR2675, CUR2676, CUR2677, CUR2681 respectively. RNA samples in duplo from each 3 transefections were pooled and 2 μg total RNA was loaded on the gel. All compounds show to be effective inhibitors. It should also be noted that the inhibition occurs at very low compound concentration.

FIG. 3 shows TRX Northern Blot of total RNA from MCF7cells treated with 4 oligomeric compounds of the invention. RNA samples in duplo from each 3 transefections were pooled and 2 μg total RNA was loaded on the gel. All compounds show to be effective inhibitors. It should also be noted that the inhibition occurs at very low compound concentration.

FIG. 4 General scheme of the synthesis of thio LNA

FIG. 5 Target sequences according to the invention; GenBank accession number, BD132005 incorporated herein as SEQ ID NO: 1, NM 003329 incorporated herein as SEQ ID NO: 2, D28376 incorporated herein as SEQ ID NO: 3, AF 548001 incorporated herein as SEQ ID NO: 4.

FIG. 6 The time course of thioredoxin protein reduction (Western blotting) in CUR2675 transfected 15PC3 cells shows constant low levels of protein, while the mock transfected cells show a strong increase of thioredoxin (upper panel). After transfection, cells were incubated in serum-containing medium for 24, 48 and 72 hours. Lower panel shows relative quantification of the thioredoxin forom the Western blotting signals. Thioredoxin data were normalised with the corresponding tubulin data.

FIG. 7 The time course of thioredoxin protein reduction (Western blotting) in CUR2676 transfected 15PC3 cells shows constant low levels of protein, while the mock transfected cells show a strong increase of thioredoxin (upper panel). Western blotting of protein extracts from transfected 15PC3 cells. After transfection, cells were incubated in serum-containing medium for 24, 48 and 72 hours. Lower panel shows relative quantification of the thioredoxin forom the Western blotting signals. Thioredoxin data were normalised with the corresponding tubulin data.

FIG. 8 Specificity of LNA oligomeric compounds targeting TRX. 15PC3 cells were transfected with LNA oligos targeting either human survivin (4LNA/PS+8PS+4LNA/PS) (named LNA survivin) or human thioredoxin (CUR2766) at 5 nM and 25 nM. The transcript steady states for TRX and Survivin. Transcript steady state was monitored by Real-time PCR and normalised to the GAPDH transcript steady state. This showed no effect of the antisense oligos targeting survivin on the TRX expression and vice versa.

FIG. 9 Apoptosis induction by LNA antisense oligomeric compounds CUR2675, CUR2768, CUR2766 and CUR2766 targeting Trx

FIG. 10 In vivo inhibition of tumour growth by CUR2681 administered 10 and 20 mg/kg s.c. day 7-20 by osmotic mini pumps. HT29, human colon cancer xenograft, BALB/c female nude mice. Mean/SEM.

### DESCRIPTION OF THE INVENTION

As used herein, the terms “target nucleic acid” encompass DNA encoding the thioredoxin or thioredoxin reductase, preferably human thioredoxin 1 (TRX1) hereafter only called TRX, and RNA (including pre-mRNA and mRNA) transcribed from such DNA, and also cDNA derived from such RNA.

As used herein, the term “gene” means the gene including exons, introns, non-coding 5′ and 3′ regions and regulatory elements and all currently known variants thereof and any further variants, which may be elucidated.

In the present context, the term “nucleoside” is used in its normal meaning, i.e. it contains a 2-deoxyribose unit or a ribose unit which is bonded through its number one carbon atom to one of the nitrogenous bases adenine (A), cytosine (C), thymine (T), uracil (U) or guanine (G).

In a similar way, the term “nucleotide” means a 2-deoxyribose unit or RNA unit which is bonded through its number one carbon atom to one of the nitrogenous bases adenine (A), cytosine (C), thymine (T) or guanine (G), uracil (U) and which is bonded through its number five carbon atom to an internucleoside phosphate group, or to a terminal group.

When used herein, the term “nucleotide analogue” refers to a non-natural occurring nucleotide wherein either the ribose unit is different from 2-deoxyribose or RNA and/or the nitrogenous base is different from A, C, T and G and/or the internucleoside phosphate linkage group is different. Specific examples of nucleoside analogues are described by e.g. Freier & Altmann; Nucl. Acid Res., 1997, 25, 4429-4443 and Uhlmann; Curr. Opinion in Drug Development, 2000, 3(2), 293-213.

The terms “corresponding nucleoside analogue” and “corresponding nucleoside” are intended to indicate that the nucleobase in the nucleoside analogue and the nucleoside is identical. For example, when the 2-deoxyribose unit of the nucleotide is linked to an adenine, the “corresponding nucleoside analogue” contains a pentose unit (different from 2-deoxyribose) linked to an adenine.

The term “nucleic acid” is defined as a molecule formed by covalent linkage of two or more nucleotides. The terms “nucleic acid” and “polynucleotide” are used interchangeable herein

The term “nucleic acid analogue” refers to a non-natural nucleic acid binding compound.

Nucleotide analogues and nucleic acid analogues are described in e.g. Freier & Altmann (Nucl. Acid Res., 1997, 25, 4429-4443) and Uhlmann (Curr. Opinion in Drug & Development (2000, 3(2): 293-213). Scheme 1 illustrates selected examples of nucleotide analogues suitable for making nucleic acids.

The term “LNA” refers to a nucleotide containing one bicyclic nucleoside analogue, also referred to as a LNA monomer, or an oligonucleotide containing one or more bicyclic nucleoside analogues. LNA monomers are described in WO 9914226 and subsequent applications, WO0056746, WO0056748, WO0066604, WO00125248, WO0228875, WO2002094250 and PCT/DK02/00488. One particular example of a thymidine LNA monomer is the (1S, 3R, 4R, 7S)-7-hydroxy-1-hydroxymethyl-5-methyl-3-(thymin-1-yl)-2,5-dioxa-bicyclo[2:2:1]heptane.

The term “oligonucleotide” refers, in the context of the present invention, to an oligomer (also called oligo) or nucleic acid polymer (e.g. ribonucleic acid (RNA) or deoxyribonucleic acid (DNA)) or nucleic acid analogue of those known in the art, preferably Locked Nucleic Acid (LNA), or a mixture thereof. This term includes oligonucleotides composed of naturally occurring nucleobases, sugars and internucleoside (backbone) linkages as well as oligonucleotides having non-naturally-occurring portions which function similarly or with specific improved functions. A fully or partly modified or substituted oligonucleotides are often preferred over native forms because of several desirable properties of such oligonucleotides such as for instance, the ability to penetrate a cell membrane, good resistance to extra- and intracellular nucleases, high affinity and specificity for the nucleic acid target. The LNA analogue is particularly preferred exhibiting the above-mentioned properties.

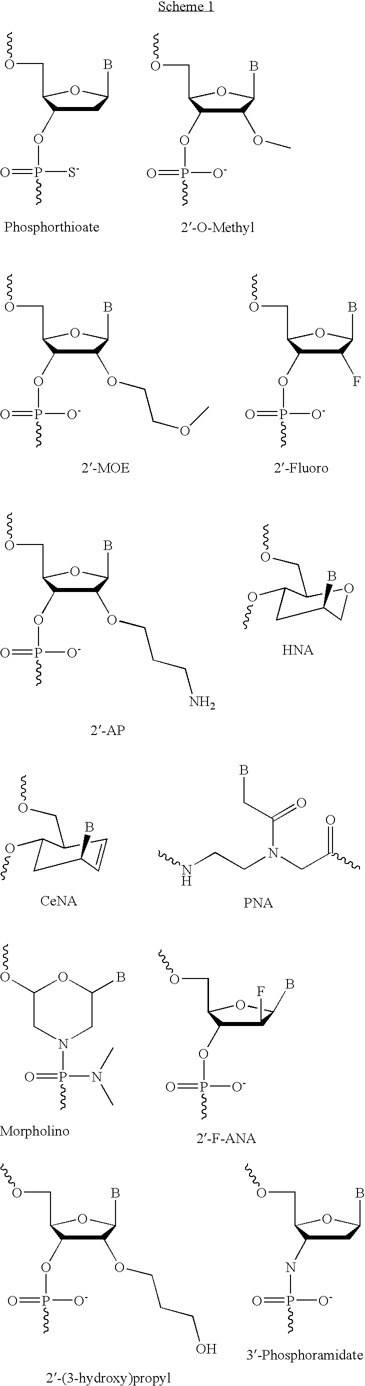

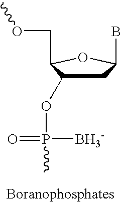

By the term “unit” is understood a monomer.

The term “at least one” comprises the integers larger than or equal to 1, such as 1, 2, 3, 4, 5, 6, 7, 8, 9, 10, 11, 12, 13, 14, 15, 16, 17 and so forth.

The term “thio-LNA” comprises a locked nucleotide in which at least one of X or Y in Scheme 2 is selected from S or —CH₂—S—. Thio-LNA can be in both beta-D and alpha-L-configuration.

The term “amino-LNA” comprises a locked nucleotide in which at least one of X or Y in Scheme 2 —N(H)—, N(R)—, CH₂—N(H)—, —CH₂—N(R)— where R is selected form hydrogen and C₁₋₄-alkyl. Amino-LNA can be in both beta-D and alpha-L-configuration.

The term “oxy-LNA” comprises a locked nucleotide in which at least one of X or Y in Scheme 2 represents —O— or —CH₂—O—. Oxy-LNA can be in both beta-D and alpha-L-configuration.

The term “ena-LNA” comprises a locked nucleotide in which Y in Scheme 2 is —CH₂—O—.

By the term “alpha-L-LNA” comprises a locked nucleotide represented as shown in Scheme 3 (structure to the right).

By the term “LNA derivatives” comprises all locked nucleotide in Scheme 2 except beta-D-methylene LNA e.g. thio-LNA, amino-LNA, alpha-L-oxy-LNA and ena-LNA.

The term “linkage group” is intended to mean a group capable of covalently coupling together two nucleosides, two nucleoside analogues, a nucleoside and a nucleoside analogue, etc. Specific and preferred examples include phosphate groups and phosphorothioate groups.

In the present context the term “conjugate” is intended to indicate a heterogenous molecule formed by the covalent attachment of a compound as described herein (i.e. a compound comprising a sequence of nucleosides or nucleoside analogues) to one or more non-nucleotide or non-polynucleotide moieties. Examples of non-nucleotide or non-polynucleotide moieties include macromolecular agents such as proteins, fatty acid chains, sugar residues, glycoproteins, polymers, or combinations thereof. Typically proteins may be antibodies for a target protein. Typical polymers may be polyethelene glycol.

The term “carcinoma” is intended to indicate a malignant tumor of epithelial origin. Epithelial tissue covers or lines the body surfaces inside and outside the body. Examples of epithelial tissue are the skin and the mucosa and serosa that line the body cavities and internal organs, such as intestines, urinary bladder, uterus, etc. Epithelial tissue may also extend into deeper tissue layers to from glands, such as mucus-secreting glands.

The term “sarcoma” is intended to indicate a malignant tumor growing from connective tissue, such as cartilage, fat, muscles, tendons and bones.

The term “glioma”, when used herein, is intended to cover a malignant tumor originating from glial cells

The term “a” as used about a nucleoside, a nucleoside analogue, a SEQ ID NO, etc. is intended to mean one or more. In particular, the expression “a component (such as a nucleoside, a nucleoside analogue, a SEQ ID NO or the like) selected from the group consisting of . . . ” is intended to mean that one or more of the cited components may be selected. Thus, expressions like “a component selected from the group consisting of A, B and C” is intended to include all combinations of A, B and C, i.e. A, B, C, A+B, A+C, B+C and A+B+C.

In the present context, the term “C₁₋₄-alkyl” is intended to mean a linear or branched saturated hydrocarbon chain wherein the chain has from one to four carbon atoms, such as methyl, ethyl, n-propyl, isopropyl, n-butyl, isobutyl, sec-butyl and tert-butyl.

As used herein, the terms “target nucleic acid” encompass DNA encoding the survivin, RNA (including pre-mRNA and mRNA) transcribed from such DNA, and also cDNA derived from such RNA.

As used herein, the terms “oligomeric compound” refers to an oligonucleotide which can induce a desired therapeutic effect in humans through for example binding by hydrogen bonding to either a target gene “Chimeraplast” and “TFO”, to the RNA transcript(s) of the target gene “antisense inhibitors”, “siRNA”, “ribozymes” and oligozymes” or to the protein(s) encoding by the target gene “aptamer”, spiegelmer” or “decoy”.

As used herein, the term “mRNA” means the presently known mRNA transcript(s) of a targeted gene, and any further transcripts, which may be identified.

As used herein, the term “modulation” means either an increase (stimulation) or a decrease (inhibition) in the expression of a gene. In the present invention, inhibition is the preferred form of modulation of gene expression and mRNA is a preferred target.

As used herein, the term “targeting” an antisense compound to a particular target nucleic acid means providing the antisense oligonucleotide to the cell, animal or human in such a way that the antisense compound are able to bind to and modulate the function of its intended target.

As used herein, “hybridisation” means hydrogen bonding, which may be Watson-Crick, Holstein, reversed Holstein hydrogen bonding, etc. between complementary nucleoside or nucleotide bases. Watson and Crick showed approximately fifty years ago that deoxyribo nucleic acid (DNA) is composed of two strands which are held together in a helical configuration by hydrogen bonds formed between opposing complementary nucleobases in the two strands. The four nucleobases, commonly found in DNA are guanine (G), adenine (A), thymine (T) and cytosine (C) of which the G nucleobase pairs with C, and the A nucleobase pairs with T. In RNA the nucleobase thymine is replaced by the nucleobase uracil (U), which similarly to the T nucleobase pairs with A. The chemical groups in the nucleobases that participate in standard duplex formation constitute the Watson-Crick face. Hoogsteen showed a couple of years later that the purine nucleobases (G and A) in addition to their Watson-Crick face have a Hoogsteen face that can be recognised from the outside of a duplex, and used to bind pyrimidine oligonucleotides via hydrogen bonding, thereby forming a triple helix structure.

In the context of the present invention “complementary” refers to the capacity for precise pairing between two nucleotides or nucleoside sequences with one another. For example, if a nucleotide at a certain position of an oligonucleotide is capable of hydrogen bonding with a nucleotide at the corresponding position of a DNA or RNA molecule, then the oligonucleotide and the DNA or RNA are considered to be complementary to each other at that position. The DNA or RNA and the oligonucleotide are considered complementary to each other when a sufficient number of nucleotides in the oligonucleotide can form hydrogen bonds with corresponding nucleotides in the target DNA or RNA to enable the formation of a sTable complex. To be stable in vitro or in vivo the sequence of an antisense compound need not be 100% complementary to its target nucleic acid. The terms “complementary” and “specifically hybridisable” thus imply that the antisense compound binds sufficiently strongly and specifically to the target molecule to provide the desired interference with the normal function of the target whilst leaving the function of non-target mRNAs unaffected.

The present invention employs oligomeric compounds, particularly antisense oligonucleotides, for use in modulating the function of nucleic acid molecules encoding TRX. The modulation is ultimately a change in the amount of TRX produced. In one embodiment this is accomplished by providing antisense compounds, which specifically hybridise with nucleic acids encoding TRX. The modulation is preferably an inhibition of the expression of TRX, which leads to a decrease in the number of functional proteins produced.

Antisense and other oligomeric compounds of the invention, which modulate expression of the target, are identified through experimentation or though rational design based on sequence information on the target and know-how on how best to design an oligomeric compound against a desired target. The sequences of these compounds are preferred embodiments of the invention. Likewise, the sequence motifs in the target to which these preferred oligomeric compounds are complementary (referred to as “hot spots”) are preferred sites for targeting.

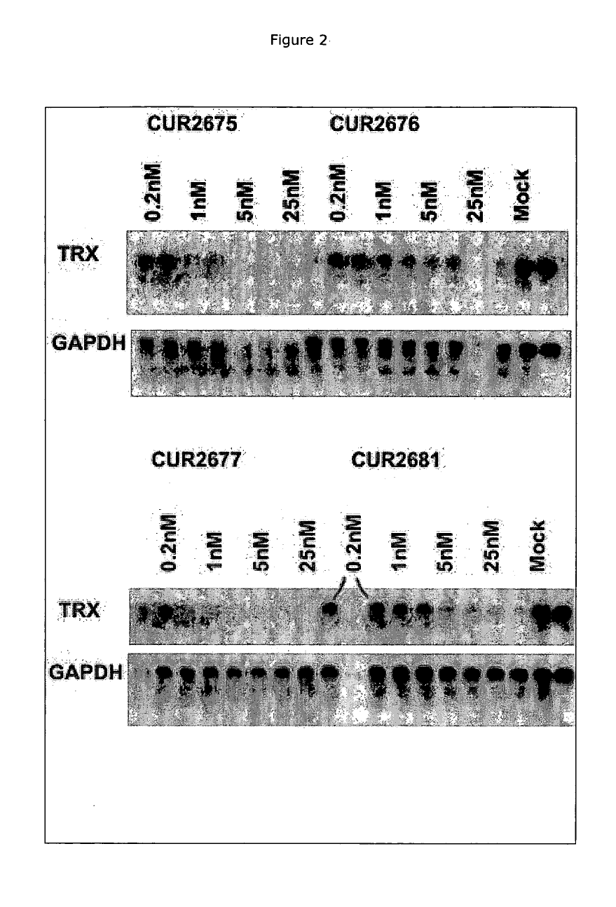

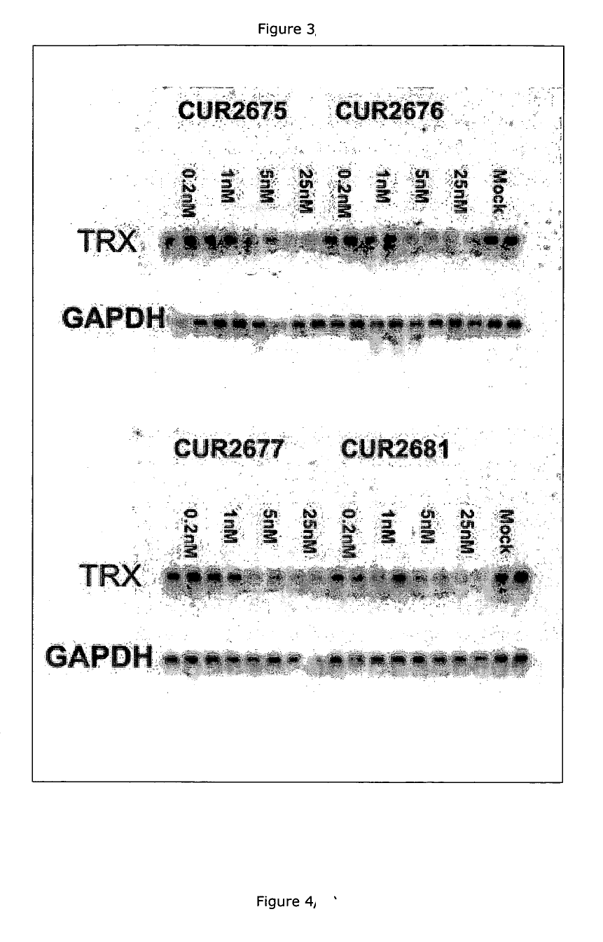

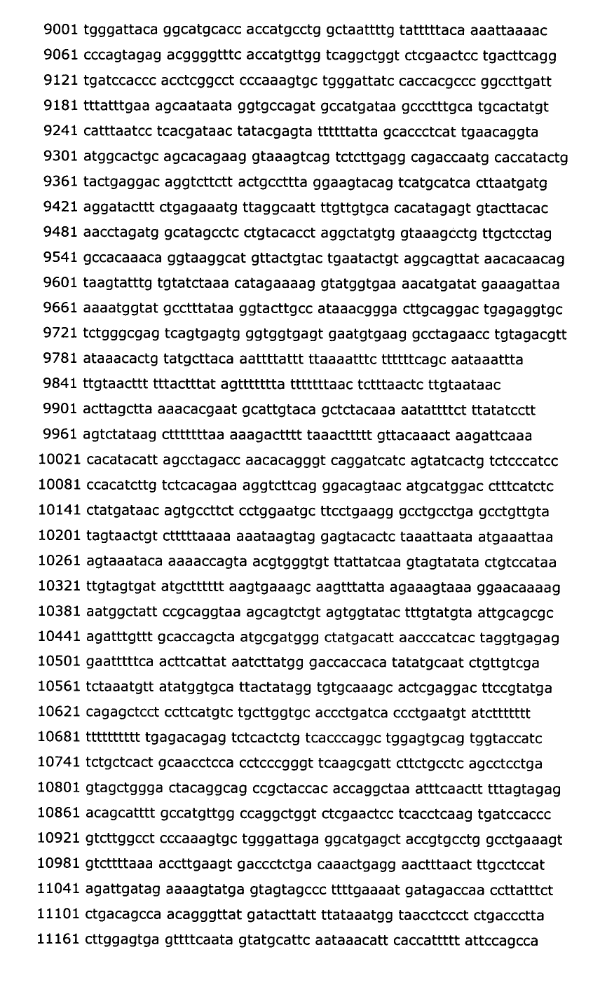

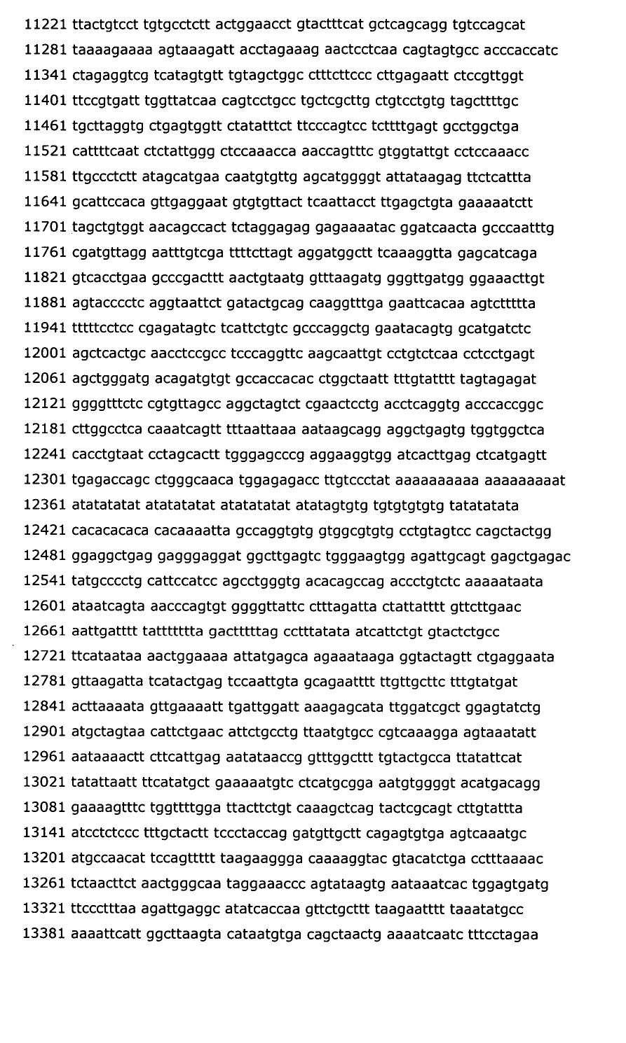

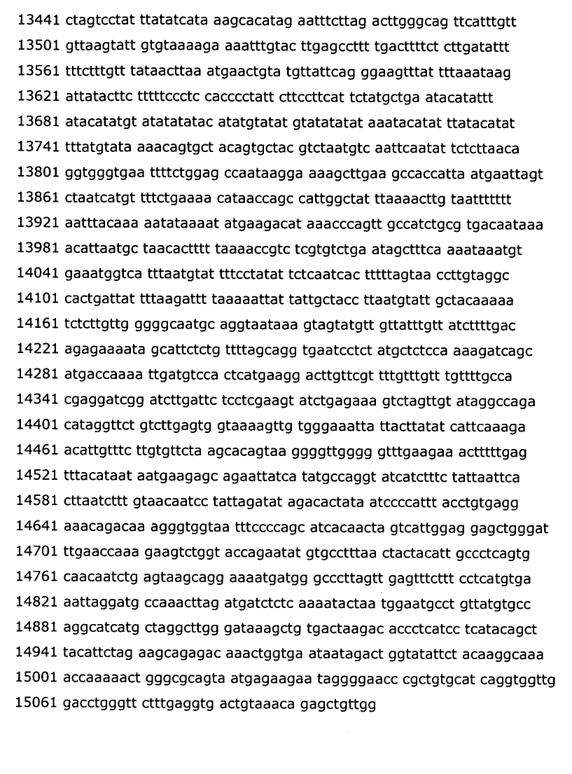

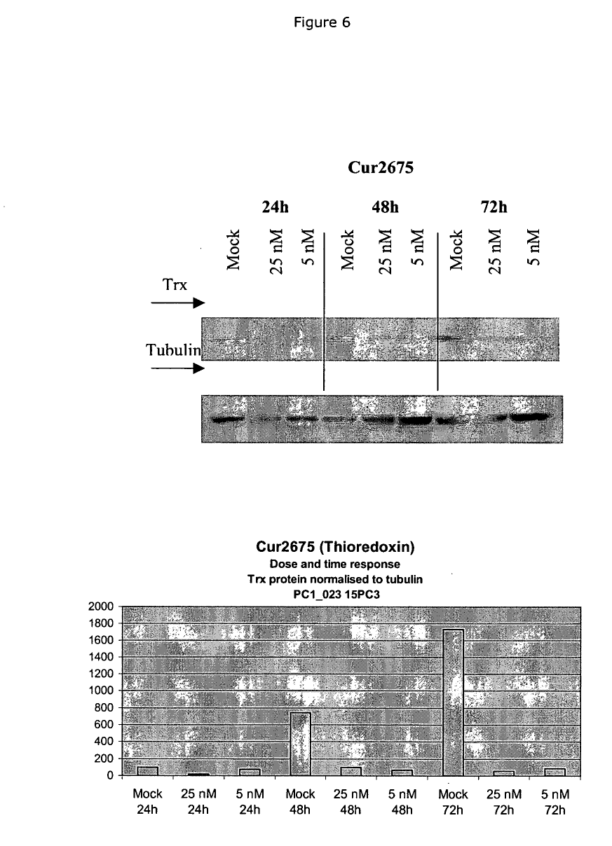

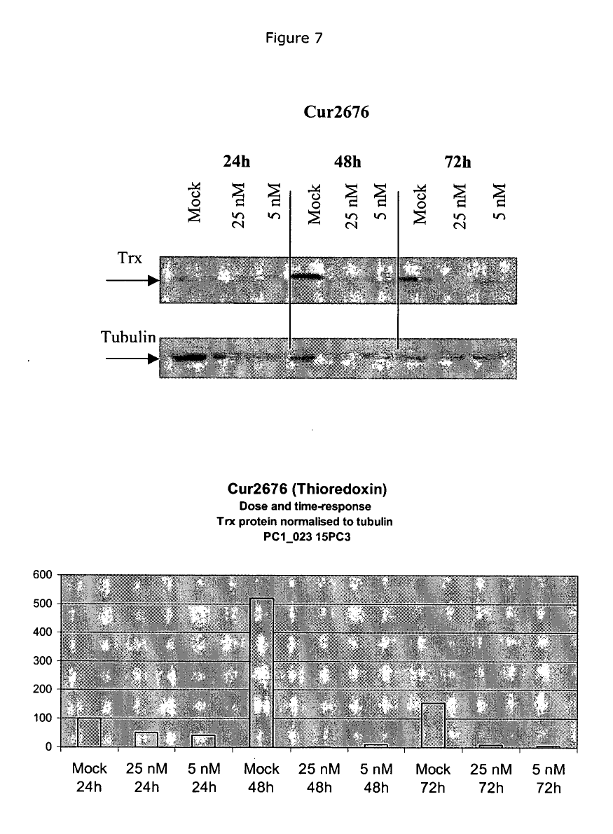

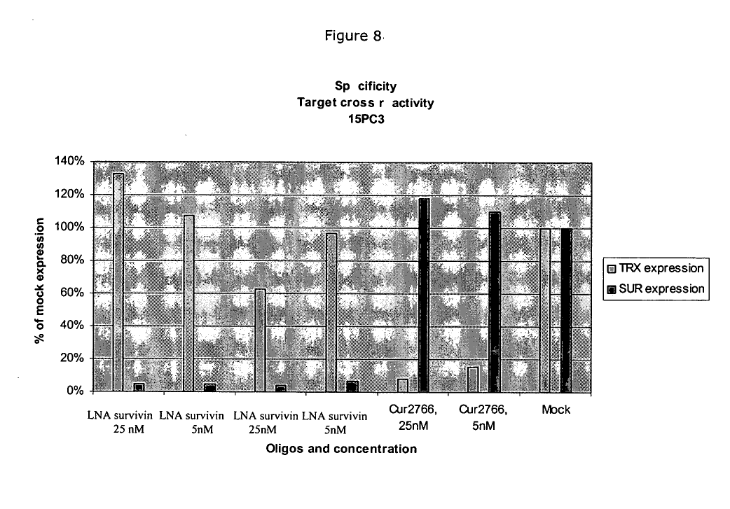

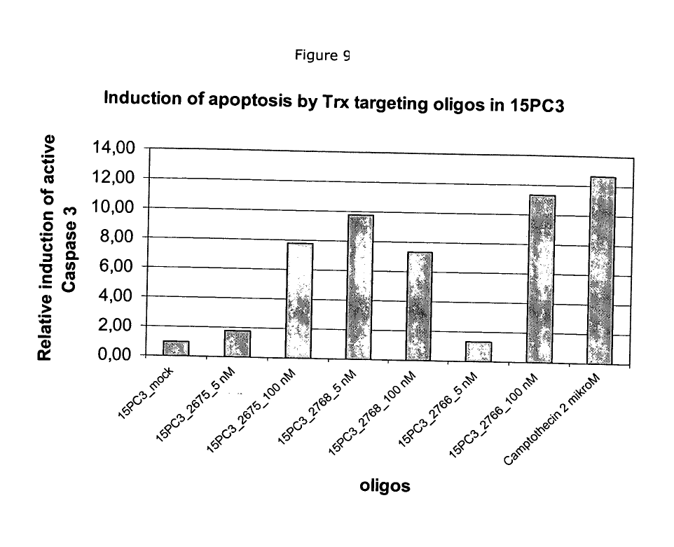

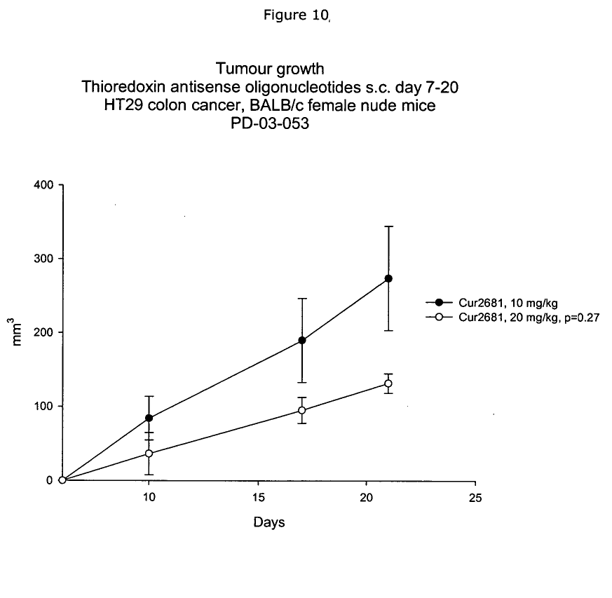

Preferred oligomeric compounds comprises at least a 8-nucleobase portion, said subsequence being selected from SEQ ID NO 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56 or 57 and their sequences are presented in table 2. The oligomeric compounds according to the invention are potent modulators of target. For example, in vitro inhibition of target is shown in Table 2 measured by Real time PCR. Low IC50 of oligomeric compounds is shown in table 3. FIG. 2 and 3 shows in vitro potency of oligomeric compounds according to the invention measured by Northern Blot. FIG. 6 and 7 shows in vitro potency of oligomeric compounds according to the invention measured by Western Blotting. FIG. 8 shows specific inhibition of a LNA oligomeric compound when monitored with another target. The compound of the invention also induces apoptosis (FIG. 9). FIG. 10 show in vivo potency of the oligomeric compounds of the invention. All the above-mentioned experimental observations show that the compounds according to the invention can constitute the active compound in a pharmaceutical composition.

In one embodiment the nucleobase portion is selected from at least 9, least 10, least 11, least 12, least 13, least 14 and least 15.

Preferred oligomeric compounds according to the invention are SEQ ID NO 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, and 57 and their sequences are presented in Table 2.

In another embodiment of the invention, said nucleosides are linked to each other by means of a phosphorothioate group. An interesting embodiment of the invention is directed to compounds of SEQ NO 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, and 57 wherein each linkage group within each compound is a phosphorothioate group. Such modifications is denoted by the subscript S. Alternatively stated, one aspect of the invention is directed to compounds of SEQ NO 5_{A}, 6_{S}, 7_{S}, 8_{S}, 9_{A}, 10_{A}, 11_{A}, 12_{A}, 13_{A}, 14_{A}, 15_{A}, 16_{A}, 17_{A}, 18_{A}, 19_{A}, 20_{A}, 21_{A}, 22_{A}, 23_{A}, 24_{A}, 25_{A}, 26_{A}, 27_{A}, 28_{A}, 29_{A}, 30_{A}, 31_{A}, 32_{A}, 33_{A}, 34_{A}, 35_{A}, 36_{A}, 37_{A}, 38_{A}, 39_{A}, 40_{A}, 41_{A}, 42_{A}, 43_{A}, 44_{A}, 45_{A}, 46_{A}, 47_{A}, 48_{A}, 49_{A}, 50_{A}, 51_{A}, 52_{A}, 53_{A}, 54_{A}, 55_{A}, 56_{A} and 57_{A}.

A further aspect of the invention is directed to compounds of SEQ NO 5_{B}, 6_{S}, 7_{S}, 8_{B}, 9_{B}, 10_{B}, 11_{B}, 12_{B}, 13_{B}, 14_{B}, 15_{B}, 16_{B}, 17_{B}, 18_{B}, 19_{B}, 20_{B}, 21_{B}, 22_{B}, 23_{B}, 24_{B}, 25_{B}, 26_{B}, 27_{B}, 28_{B}, 29_{B}, 30_{B}, 31_{B}, 32_{B}, 33_{B}, 34_{B}, 35_{B}, 36_{B}, 37_{S}, 38_{B}, 39_{B}, 40_{B}, 41_{B}, 42_{B}, 43_{B}, 44_{B}, 45_{B}, 46_{B}, 47_{B}, 48_{B}, 49_{B}, 50_{B}, 51_{B}, 52_{B}, 53_{B}, 54_{B}, 55_{B}, 56_{B}, and 57_{B}.

A further aspect of the invention is directed to compounds of SEQ NO 5_{C}, 6_{S}, 7_{S}, 8_{C}, 9_{C}, 10_{C}, 11_{C}, 12_{C}, 13_{C}, 14_{C}, 15_{C}, 16_{C}, 17_{C}, 18_{C}, 19_{C}, 20_{C}, 21_{C}, 22_{C}, 23_{C}, 24_{C}, 25_{C}, 26_{C}, 27_{C}, 28_{C}, 29_{C}, 30_{C}, 31_{C}, 32_{C}, 33_{C}, 34_{C}, 35_{C}, 36_{C}, 37_{C}, 38_{S}, 39_{C}, 40_{C}, 41_{C}, 42_{C}, 43_{C}, 44_{C}, 45_{C}, 46_{C}, 47_{C}, 48_{C}, 49_{C}, 50_{C}, 51_{C}, 52_{C}, 53_{C}, 54_{C}, 55_{C}, 56_{C}, and 57_{C}.

In one embodiment of the invention the oligomeric compounds are containing at least on unit of chemistry termed LNA (Locked Nucleic Acid).

LNA monomer typically refers to a bicyclic nucleoside analogue, as described in the International Patent Application WO 99/14226 and subsequent applications, WO0056746, WO0056748, WO0066604, WO00125248, WO0228875, WO2002094250 and PCT/DK02/00488 all incorporated herein by reference. Preferred LNA monomers structures are exemplified in Scheme 2

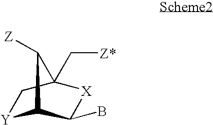

X and Y are independently selected among the groups —O—, —S—, —N(H)—, N(R)—, —CH₂— or —CH— (if part of a double bond), —CH₂—O—, —CH₂—S—, —CH₂—N(H)—, —CH₂—N(R)—, —CH₂—CH₂— or —CH₂—CH— (if part of a double bond), —CH═CH—, where R is selected form hydrogen and C₁₋₄-alkyl. The asymmetric groups may be found in either orientation. In Scheme 2, the 4 chiral centers are shown in a fixed configuration. However, the configuarations in Scheme 2 are not necessarily fixed. Also comprised in this invention are compounds of the general Scheme 2 in which the chiral centers are found in different configurations, such as those represented in Scheme 3 or 4. Thus, the intention in the illustration of Scheme 2 is not to limit the configuration of the chiral centre. Each chiral center in Scheme 2 can exist in either R or S configuration. The definition of R (rectus) and S (sinister) are described in the IUPAC 1974 Recommendations, Section E, Fundamental Stereochemistry: The rules can be found in Pure Appl. Chem. 45, 13-30, (1976) and in “Nomenclature of organic Chemistry” pergamon, New York, 1979.

Z and Z* are independently absent, selected among an internucleoside linkage, a terminal group or a protecting group.

The internucleoside linkage may be —O—P(O)₂—O—, —O—P(O,S)—O—, —O—P(S)₂—O—, —S—P(O)₂—, O—, —S—P(O,S)—O—, —S—P(S)₂—O—, —O—P(O)₂—S—, —O—P(O,S)—S—, —S—P(O)₂—S—, —O—PO(R^{H})—O—, O—PO(OCH₃)—O—, —O—PO(NR^{H})—O—, —O—PO(OCH₂CH₂S—R)—O—, —O—PO(BH₃)—O—, —O—PO(NHR^{H})—O—, —O—P(O)₂—NR^{H}, —NR^{H}—P(O)₂—O—, —NR^{H}—CO—O—, —NR^{H}—CO—NR^{H}—, —O—CO—O—, —O—CO—NR^{H}—, —NR^{H}—CO—CH₂—, —O—CH₂—CO—NR^{H}—, —O—CH₂—CH₂—NR^{H}—, —CO—NR^{H}—CH₂—, —CH₂—NR^{H}—CO—, —O—CH₂—CH₂—S—, —S—CH₂—CH₂—O—, —S—CH₂—CH₂—S—, —CH₂—SO₂—CH₂—, —CH₂—CO—NR^{H}—, —O—CH₂—CH₂—NR^{H}—CO—, —CH₂—NCH₃—O—CH₂—, where R^{H} is selected form hydrogen and C₁₋₄-alkyl,

The terminal groups are selected independently among from hydrogen, azido, halogen, cyano, nitro, hydroxy, Prot-O—, Act-O—, mercapto, Prot-S—, Act-S—, C₁₋₆-alkylthio, amino, Prot-N(R^{H})—, Act-N(R^{H})—, mono- or di(C₁₋₆-alkyl)amino, optionally substituted C₁₋₆-alkoxy, optionally substituted C₁₋₆-alkyl, optionally substituted C₂₋₆-alkenyl, optionally substituted C₂₋₆-alkenyloxy, optionally substituted C₂₋₆-alkynyl, optionally substituted C₂₋₆-alkynyloxy, monophosphate, monothiophosphate, diphosphate, dithiophosphate triphosphate, trithiophosphate, DNA intercalators, photochemically active groups, thermochemically active groups, chelating groups, reporter groups, ligands, carboxy, sulphono, hydroxymethyl, Prot-O—CH₂—, Act-O—CH₂—, aminomethyl, Prot-N(R^{H})—CH₂—, Act-N(R^{H})—CH₂—, carboxymethyl, sulphonomethyl, where Prot is a protection group for —OH, —SH, and —NH(R^{H}), respectively, Act is an activation group for —OH, —SH, and —NH(R^{H}), respectively, and R^{H} is selected from hydrogen and C₁₋₆-alkyl.

The protection groups of hydroxy substituents comprises substituted trityl, such as 4,4′-dimethoxytrityloxy (DMT), 4-monomethoxytrityloxy (MMT), and trityloxy, optionally substituted 9-(9-phenyl)xanthenyloxy (pixyl), optionally substituted methoxytetrahydro-pyranyloxy (mthp), silyloxy such as trimethylsilyloxy (TMS), triisopropylsilyloxy (TIPS), tert-butyldimethylsilyloxy (TBDMS), triethylsilyloxy, and phenyidimethylsilyloxy, tert-butylethers, acetals (including two hydroxy groups), acyloxy such as acetyl or halogen substituted acetyls, e.g. chloroacetyloxy or fluoroacetyloxy, isobutyryloxy, pivaloyloxy, benzoyloxy and substituted benzoyls, methoxymethyloxy (MOM), benzyl ethers or substituted benzyl ethers such as 2,6-dichlorobenzyloxy (2,6-Cl₂Bzl). Alternatively when Z or Z* is hydroxyl they may be protected by attachment to a solid support optionally through a linker.

When Z or Z* is amino groups illustrative examples of the amino protection protections are fluorenylmethoxycarbonylamino (Fmoc), tert-butyloxycarbonylamino (BOC), trifluoroacetylamino, allyloxycarbonylamino (alloc, AOC), Z benzyloxycarbonylamino (Cbz), substituted benzyloxycarbonylaminos such as 2-chloro benzyloxycarbonylamino (2-ClZ), monomethoxytritylamino (MMT), dimethoxytritylamino (DMT), phthaloylamino, and 9-(9-phenyl)xanthenylamino (pixyl).

In the embodiment above, Act designates an activation group for —OH, —SH, and —NH(R^{H}), respectively. Such activation groups are, e.g., selected from optionally substituted O-phosphoramidite, optionally substituted O-phosphortriester, optionally substituted O-phosphordiester, optionally substituted H-phosphonate, and optionally substituted O-phosphonate.

In the present context, the term “phosphoramidite” means a group of the formula —P(OR^{x})—N(R^{y})₂, wherein R^{x} designates an optionally substituted alkyl group, e.g. methyl, 2-cyanoethyl, or benzyl, and each of R^{y} designate optionally substituted alkyl groups, e.g. ethyl or isopropyl, or the group —N(R^{y})₂ forms a morpholino group (—N(CH₂CH₂)₂O). R^{x} preferably designates 2-cyanoethyl and the two R^{y} are preferably identical and designate isopropyl. Thus, an especially relevant phosphoramidite is N,N-diisopropyl-O-(2-cyanoethyl)phosphoramidite.

B constitutes a natural or non-natural nucleobase and selected among adenine, cytosine, 5-methylcytosine, isocytosine, pseudoisocytosine, guanine, thymine, uracil, 5-bromouracil, 5-propynyluracil, 5-propyny-6-fluoroluracil, 5-methylthiazoleuracil, 6-aminopurine, 2-aminopurine, inosine, diaminopurine, 7-propyne-7-deazaadenine, 7-propyne-7-deazaguanine, 2-chloro-6-aminopurine.

Particularly preferred bicyclic structures are shown in Scheme 3 below:

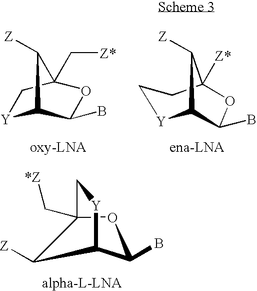

Where Y is independently selected from —O—, —S—, —NH—, and N(R^{H}), Z and Z* are independently selected among an internucleoside linkage, a terminal group or a protecting group.

The internucleoside linkage may be —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —S—P(O)₂—S—, —O—PO(R^{H})—O—, O—PO(OCH₃)—O—, —O—PO(NR^{H})—O—, —O—PO(OCH₂CH₂S—R)—O—, —O—PO(BH₃)—O—, —O—PO(NHR^{H})—O—, —O—P(O)₂—NR^{H}—, —NR^{H}—P(O)₂—O—, —NR^{H}—CO—O—, where R^{H} is selected form hydrogen and C₁₋₄-alkyl.

The terminal groups are selected independently among from hydrogen, azido, halogen, cyano, nitro, hydroxy, Prot-O—, Act-O—, mercapto, Prot-S—, Act-S—, C₁₋₆-alkylthio, amino, Prot-N(R^{H})—, Act-N(R^{H})—, mono- or di(C₁₋₆-alkyl)amino, optionally substituted C₁₋₆-alkoxy, optionally substituted C₁₋₆-alkyl, optionally substituted monophosphate, monothiophosphate, diphosphate, dithiophosphate triphosphate, trithiophosphate, where Prot is a protection group for —OH, —SH, and —NH(R^{H}), respectively, Act is an activation group for —OH, —SH, and —NH(R^{H}), respectively, and R^{H} is selected from hydrogen and C₁₋₆-alkyl.

The protection groups of hydroxy substituents comprises substituted trityl, such as 4,4′-dimethoxytrityloxy (DMT), 4-monomethoxytrityloxy (MMT), optionally substituted 9-(9-phenyl)xanthenyloxy (pixyl), optionally substituted methoxytetrahydropyranyloxy (mthp), silyloxy such as trimethylsilyloxy (TMS), triisopropylsilyloxy (TIPS), tert-butyl-dimethylsilyloxy (TBDMS), triethylsilyloxy, and phenyldimethylsilyloxy, tert-butylethers, acetals (including two hydroxy groups), acyloxy such as acetyl Alternatively when Z or Z* is hydroxyl they may be protected by attachment to a solid support optionally through a linker.

When Z or Z* is amino groups illustrative examples of the amino protection protections are fluorenylmethoxycarbonylamino (Fmoc), tert-butyloxycarbonylamino (BOC), trifluoroacetylamino, allyloxycarbonylamino (alloc, AOC), monomethoxytritylamino (MMT), dimethoxytritylamino (DMT), phthaloylamino.

In the embodiment above, Act designates an activation group for —OH, —SH, and —NH(R^{H}), respectively. Such activation groups are, e.g., selected from optionally substituted O-phosphoramidite, optionally substituted O-phosphortriester, optionally substituted O-phosphordiester, optionally substituted H-phosphonate, and optionally substituted O-phosphonate.

In the present context, the term “phosphoramidite” means a group of the formula —P(OR^{x})—N(R^{y})₂, wherein R^{x} designates an optionally substituted alkyl group, e.g. methyl, 2-cyanoethyl, and each of R^{y} designate optionally substituted alkyl groups, R^{x} preferably designates 2-cyanoethyl and the two R^{y} are preferably identical and designate isopropyl. Thus, an especially relevant phosphoramidite is N,N-diisopropyl-O-(2-cyanoethyl)-phosphoramidite.

B constitutes a natural or non-natural nucleobase and selected among adenine, cytosine, 5-methylcytosine, isocytosine, pseudoisocytosine, guanine, thymine, uracil, 5-bromouracil, 5-propynyluracil, 6-aminopurine, 2-aminopurine, inosine, diaminopurine, 2-chloro-6-aminopurine.

Specifically preferred LNA units are shown in scheme 4.

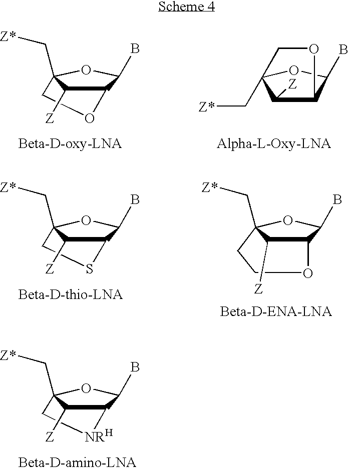

Therapeutic Principle

A person skilled in the art will appreciate that oligomeric compounds containing LNA can be used to combat TRX linked diseases by many different principles, which thus falls within the spirit of the present invention.

For instance, LNA oligomeric compounds may be designed as antisense inhibitors, which are single stranded nucleic acids that prevent the production of a disease causing protein, by intervention at the mRNA level. Also, they may be designed as Ribozymes or Oligozymes which are antisense oligonucleotides which in addition to the target binding domain(s) comprise a catalytic activity that degrades the target mRNA (ribozymes) or comprise an external guide sequence (EGS) that recruit an endogenous enzyme (RNase P) which degrades the target mRNA (oligozymes).

Equally well, the LNA oligomeric compounds may be designed as siRNA's which are small double stranded RNA molecules that are used by cells to silence specific endogenous or exogenous genes by an as yet poorly understood “antisense-like” mechanism.

LNA oligomeric compounds may also be designed as Aptamers (and a variation thereof, termed Spiegelmers) which are nucleic acids that through intra-molecular hydrogen bonding adopt three-dimensional structures that enable them to bind to and block their biological targets with high affinity and specificity. Also, LNA oligomeric compounds may be designed as Decoys, which are small double-stranded nucleic acids that prevent cellular transcription factors from transactivating their target genes by selectively blocking their DNA binding site.

Furthermore, LNA oligomeric compounds may be designed as Chimeraplasts, which are small single stranded nucleic acids that are able to specifically pair with and alter a target gene sequence. LNA containing oligomeric compounds exploiting this principle therefore may be particularly useful for treating TRX linked diseases that are caused by a mutation in the TRX gene.

Finally, LNA oligomeric compounds may be designed as TFO's (triplex forming oligonucleotides), which are nucleic acids that bind to double stranded DNA and prevent the production of a disease causing protein, by intervention at the RNA transcription level.

Dictated in part by the therapeutic principle by which the oligonucleotide is intended to operate, the LNA oligomeric compounds in accordance with this invention preferably comprise from about 8 to about 60 nucleobases i.e. from about 8 to about 60 linked nucleosides. Particularly preferred compounds are antisense oligonucleotides comprising from about 12 to about 30 nucleobases and most preferably are antisense compounds comprising about 12-20 nucleobases.

Referring to the above principles by which an LNA oligomeric compound can elicit its therapeutic action the target of the present invention may be the TRX gene, the mRNA or the protein. In the most preferred embodiment the LNA oligomeric compounds is designed as an antisense inhibitor directed against the TRX pre-mRNA or TRX mRNA. The oligonucleotides may hybridize to any site along the TRX pre-mRNA or mRNA such as sites in the 5′ untranslated leader, exons, introns and 3′ untranslated tail.

In a preferred embodiment, the oligonucleotide hybridizes to a portion of the human TRX pre-mRNA or mRNA that comprises the translation-initiation site. More preferably, the TRX oligonucleotide comprises a CAT sequence, which is complementary to the AUG initiation sequence of the TRX pre-mRNA or RNA. In another embodiment, the TRX oligonucleotide hybridizes to a portion of the splice donor site of the human TRX pre-mRNA. In yet another embodiment, TRX oligonucleotide hybridizes to a portion of the splice acceptor site of the human TRX pre-mRNA. In another embodiment, the TRX oligonucleotide hybridizes to portions of the human TRX pre-mRNA or mRNA involved in polyadenylation, transport or degradation.

The skilled person will appreciate that preferred oligonucleotides are those that hybridize to a portion of the TRX pre-mRNA or mRNA whose sequence does not commonly occur in transcripts from unrelated genes so as to maintain treatment specificity.

The oligomeric compound of the invention are designed to be sufficiently complementary to the target to provide the desired clinical response e.g. the oligomeric compound must bind with sufficient strength and specificity to its target to give the desired effect. In one embodiment, said compound modulating TRX is designed so as to also modulate other specific nucleic acids which do not encode TRX.

It is preferred that the oligomeric compound according to the invention is designed so that intra- and intermolecular oligonucleotide hybridisation is avoided.

In many cases the identification of an LNA oligomeric compound effective in modulating TRX activity in vivo or clinically is based on sequence information on the target gene. However, one of ordinary skill in the art will appreciate that such oligomeric compounds can also be identified by empirical testing. As such TRX oligomeric compounds having, for example, less sequence homology, greater or fewer modified nucleotides, or longer or shorter lengths, compared to those of the preferred embodiments, but which nevertheless demonstrate responses in clinical treatments, are also within the scope of the invention.

Antisense Drugs

In one embodiment of the invention the oligomeric compounds are suitable antisense drugs. The design of a potent and safe antisense drug requires the fine-tuning of diverse parameters such as affinity/specificity, stability in biological fluids, cellular uptake, mode of action, pharmacokinetic properties and toxicity.

Affinity & specificity: LNA with an oxymethylene 2′-O, 4′-C linkage (β-D-oxy-LNA), exhibits unprecedented binding properties towards DNA and RNA target sequences. Likewise LNA derivatives, such as amino-, thio- and α-L-oxy-LNA display unprecedented affinities towards complementary RNA and DNA and in the case of thio-LNA the affinity towards RNA is even better than with the □-D-oxy-LNA.

In addition to these remarkable hybridization properties, LNA monomers can be mixed and act cooperatively with DNA and RNA monomers, and with other nucleic acid analogues, such as 2′-O-alkyl modified RNA monomers. As such, the oligonucleotides of the present invention can be composed entirely of β-D-oxy-LNA monomers or it may be composed of β-D-oxy-LNA in any combination with DNA, RNA or contemporary nucleic acid analogues which includes LNA derivatives such as for instance amino-, thio- and α-L-oxy-LNA. The unprecedented binding affinity of LNA towards DNA or RNA target sequences and its ability to mix freely with DNA, RNA and a range of contemporary nucleic acid analogues has a range of important consequences according to the invention for the development of effective and safe antisense compounds.

Firstly, in one embodiment of the invention it enables a considerable shortening of the usual length of an antisense oligo (from 20-25 mers to, e.g., 12-15 mers) without compromising the affinity required for pharmacological activity. As the intrinsic specificity of an oligo is inversely correlated to its length, such a shortening will significantly increase the specificity of the antisense compound towards its RNA target. One embodiment of the invention is to, due to the sequence of the humane genome is available and the annotation of its genes rapidly progressing, identify the shortest possible, unique sequences in the target mRNA.

In another embodiment, the use of LNA to reduce the size of oligos significantly eases the process and prize of manufacture thus providing the basis for antisense therapy to become a commercially competitive treatment offer for a diversity of diseases.

In another embodiment, the unprecedented affinity of LNA can be used to substantially enhance the ability of an antisense oligo to hybridize to its target mRNA in-vivo thus significantly reducing the time and effort required for identifying an active compound as compared to the situation with other chemistries.

In another embodiment, the unprecedented affinity of LNA is used to enhance the potency of antisense oligonucleotides thus enabling the development of compounds with more favorable therapeutic windows than those currently in clinical trials.

When designed as an antisense inhibitor, the oligonucleotides of the invention bind to the target nucleic acid and modulate the expression of its cognate protein. Preferably, such modulation produces an inhibition of expression of at least 10% or 20% compared to the normal expression level, more preferably at least a 30%, 40%, 50%, 60%, 70%, 80%, or 90% inhibition compared to the normal expression level.

Typically, the LNA oligonucleotides of the invention will contain other residues than □-D-oxy-LNA such as native DNA monomers, RNA monomers, N3′-P5′ phosphoroamidates, 2′-F, 2′-O-Me, 2′-O-methoxyethyl (MOE), 2′-O-(3-aminopropyl) (AP), hexitol nucleic acid (HNA), 2′-F-arabino nucleic acid (2′-F-ANA) and D-cyclohexenyl nucleoside (CeNA). Also, the β-D-oxy-LNA-modified oligonucleotide may also contain other LNA units in addition to or in place of an oxy-LNA group. In particular, preferred additional LNA units include thio-LNA or amino-LNA monomers in either the D-β or L-α configurations or combinations thereof or ena-LNA. In general, an LNA-modified oligonucleotide will contain at least about 5, 10, 15 or 20 percent LNA units, based on total nucleotides of the oligonucleotide, more typically at least about 20, 25, 30, 40, 50, 60, 70, 80 or 90 percent LNA units, based on total bases of the oligonucleotide.

Stability in biological fluids: One embodiment of the invention includes the incorporation of LNA monomers into a standard DNA or RNA oligonucleotide to increase the stability of the resulting oligomeric compound in biological fluids e.g. through the increase of resistance towards nucleases (endonucleases and exonucleases). The extent of stability will depend on the number of LNA monomers used, their position in the oligonucleotide and the type of LNA monomer used. Compared to DNA and phosphorothioates the following order of ability to stabilize an oligonucleotide against nucleolytic degradation can be established: DNA<<phosphorothioates˜oxy-LNA<α-L-LNA<amino-LNA<thio-LNA.

Given the fact that LNA is compatible with standard DNA synthesis and mixes freely with many contemporary nucleic acid analogues nuclease resistance of LNA-oligomeric compounds can be further enhanced according to the invention by either incorporating other analogues that display increased nuclease stability or by exploiting nuclease-resistant internucleoside linkages e.g. phosphoromonothioate, phosphorodithioate, and methylphosphonate linkages, etc.

Mode of action: Antisense compounds according to the invention may elicit their therapeutic action via a variety of mechanisms and may be able to combine several of these in the same compound. In one scenario, binding of the oligonucleotide to its target (pre-mRNA or mRNA) acts to prevent binding of other factors (proteins, other nucleic acids, etc.) needed for the proper function of the target i.e. operate by steric hindrance. For instance, the antisense oligonucleotide may bind to sequence motifs in either the pre-mRNA or mRNA that are important for recognition and binding of transacting factors involved in splicing, poly-adenylation, cellular transport, post-transcriptional modifications of nucleosides in the RNA, capping of the 5′-end, translation, etc. In the case of pre-mRNA splicing, the outcome of the interaction between the oligonucleotide and its target may be either suppression of expression of an undesired protein, generation of alternative spliced mRNA encoding a desired protein or both.

In another embodiment, binding of the oligonucleotide to its target disables the translation process by creating a physical block to the ribosomal machinery, i.e. tranlational arrest.

In yet another embodiment, binding of the oligonucleotide to its target interferes with the RNAs ability to adopt secondary and higher order structures that are important for its proper function, i.e. structural interference. For instance, the oligonucleotide may interfere with the formation of stem-loop structures that play crucial roles in different functions, such as providing additional stability to the RNA or adopting essential recognition motifs for different proteins.

In still another embodiment, binding of the oligonucleotide inactivates the target toward further cellular metabolic processes by recruiting cellular enzymes that degrades the mRNA. For instance, the oligonucleotide may comprise a segment of nucleosides that have the ability to recruit ribonuclease H (RNaseH) that degrades the RNA part of a DNA/RNA duplex. Likewise, the oligonucleotide may comprise a segment which recruits double stranded RNAses, such as for instance RNAseIII or it may comprise an external guide sequence (EGS) that recruit an endogenous enzyme (RNase P) which degrades the target mRNA Also, the oligonucleotide may comprise a sequence motif which exhibit RNAse catalytic activity or moieties may be attached to the oligonucleotides which when brought into proximity with the target by the hybridization event disables the target from further metabolic activities.

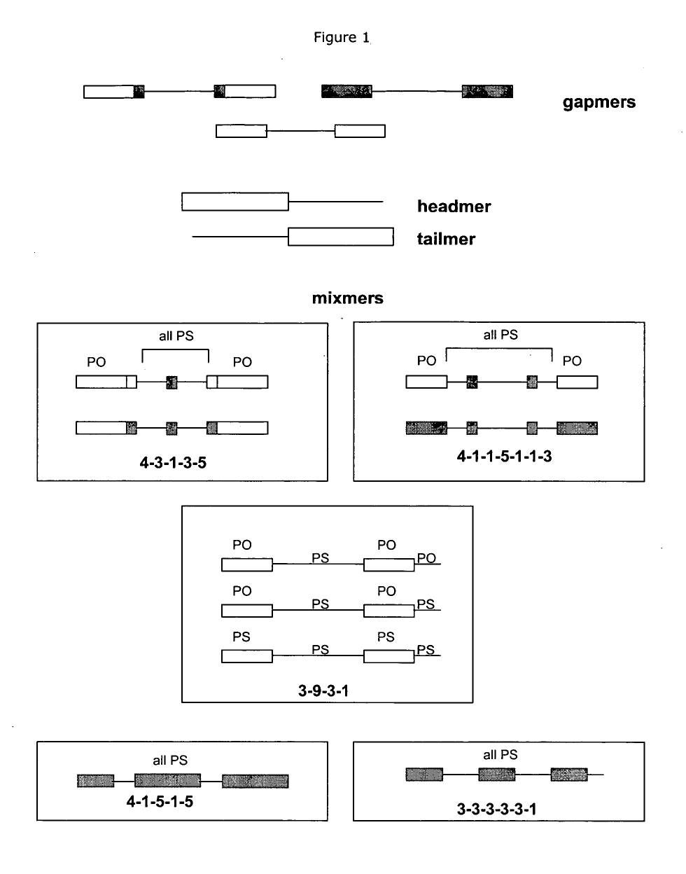

It has been shown that β-D-oxy-LNA does not support RNaseH activity. However, this can be changed according to the invention by creating chimeric oligonucleotides composed of β-D-oxy-LNA and DNA, called gapmers. A gapmer is based on a central stretch of 4-12 nt DNA or modified monomers recognizable and cleavable by the RNaseH (the gap) typically flanked by 1 to 6 residues of β-D-oxy-LNA (the flanks). The flanks can also be constructed with LNA derivatives. There are other chimeric constructs according to the invention that are able to act via an RNaseH mediated mechanism. A headmer is defined by a contiguous stretch of β-D-oxy-LNA or LNA derivatives at the 5′-end followed by a contiguous stretch of DNA or modified monomers recognizable and cleavable by the RNaseH towards the 3′-end, and a tailmer is defined by a contiguous stretch of DNA or modified monomers recognizable and cleavable by the RNaseH at the 5′-end followed by a contiguous stretch of β-D-oxy-LNA or LNA derivatives towards the 3′-end. Other chimeras according to the invention, called mixmers consisting of an alternate composition of DNA or modified monomers recognizable and cleavable by RNaseH and β-D-oxy-LNA and/or LNA derivatives might also be able to mediate RNaseH binding and cleavage. Since α-L-LNA recruits RNaseH activity to a certain extent, smaller gaps of DNA or modified monomers recognizable and cleavable by the RNaseH for the gapmer construct might be required, and more flexibility in the mixmer construction might be introduced. FIG. 1 shows an outline of different designs according to the invention.

Pharmacokinetic Properties

The clinical effectiveness of antisense oligonucleotides depends to a significant extent on their pharmacokinetics e.g. absorption, distribution, cellular uptake, metabolism and excretion. In turn these parameters are guided significantly by the underlying chemistry and the size and three-dimensional structure of the oligonucleotide.

As mentioned earlier LNA according to the invention is not a single, but several related chemistries, which although molecularly different all exhibit stunning affinity towards complementary DNA and RNA, Thus, the LNA family of chemistries are uniquely suited of development oligos according to the invention with tailored pharmacokinetic properties exploiting either the high affinity of LNA to modulate the size of the active compounds or exploiting different LNA chemistries to modulate the exact molecular composition of the active compounds. In the latter case, the use of for instance amino-LNA rather than oxy-LNA will change the overall charge of the oligo and affect uptake and distribution behavior. Likewise the use of thio-LNA instead of oxy-LNA will increase the lipophilicity of the oligonucleotide and thus influence its ability to pass through lipophilic barriers such as for instance the cell membrane.

Modulating the pharmacokinetic properties of an LNA oligonucleotide according to the invention may further be achieved through attachment of a variety of different moieties. For instance, the ability of oligonucleotides to pass the cell membrane may be enhanced by attaching for instance lipid moieties such as a cholesterol moiety, a thioether, an aliphatic chain, a phospholipid or a polyamine to the oligonucleotide. Likewise, uptake of LNA oligonucleotides into cells may be enhanced by conjugating moieties to the oligonucleotide that interacts with molecules in the membrane, which mediates transport into the cytoplasm.

Pharmacodynamic Properties

The pharmacodynamic properties can according to the invention be enhanced with groups that improve oligomer uptake, enhance biostability such as enhance oligomer resistance to degradation, and/or increase the specificity and affinity of oligonucleotides hybridisation characteristics with target sequence e.g. a mRNA sequence.

Toxicology

There are basically two types of toxicity associated with antisense oligos: sequence-dependant toxicity, involving the base sequence, and sequence-independent, class-related toxicity. With the exception of the issues related to immunostimulation by native CpG sequence motifs, the toxicities that have been the most prominent in the development of antisense oligonucleotides are independent of the sequence, e.g. related to the chemistry of the oligonucleotide and dose, mode, frequency and duration of administration. The phosphorothioates class of oligonucleotides have been particularly well characterized and found to elicit a number of adverse effects such as complement activation, prolonged PTT (partial thromboplastin time), thrombocytopenia, hepatotoxicity (elevation of liver enzymes), cardiotoxicity, splenomegaly and hyperplasia of reticuloendothelial cells.

As mentioned earlier, the LNA family of chemistries provide unprecedented affinity, very high bio-stablity and the ability to modulate the exact molecular composition of the oligonucleotide. In one embodiment of the invention, LNA containing compounds enables the development of oligonucleotides which combine high potency with little- if any- phosphorothioate linkages and which are therefore likely to display better efficacy and safety than contemporary antisense compounds.

Manufacture

Oligo- and polynucleotides of the invention may be produced using the polymerisation techniques of nucleic acid chemistry well known to a person of ordinary skill in the art of organic chemistry. Generally, standard oligomerisation cycles of the phosphoramidite approach (S. L. Beaucage and R. P. Iyer, Tetrahedron, 1993, 49, 6123; S. L. Beaucage and R. P. Iyer, Tetrahedron, 1992, 48, 2223) is used, but e.g. H-phosphonate chemistry, phosphortriester chemistry can also be used.

For some monomers of the invention longer coupling time, and/or repeated couplings with fresh reagents, and/or use of more concentrated coupling reagents were used. The phosphoramidites employed coupled with satisfactory >95% step-wise coupling yields. Thiolation of the phosphate is performed by exchanging the normal, e.g. iodine/pyridine/H₂O, oxidation used for synthesis of phosphordiester oligomers with an oxidation using Beaucage's reagent (commercially available) other sulfurisation reagents are also comprised. The phosphorthioate LNA oligomers were efficiently synthesised with stepwise coupling yields >=98%.

The β-D-amino-LNA, β-D-thio-LNA oligonucleotides, α-L-LNA and β-D-methylamino-LNA oligonucleotides were also efficiently synthesised with step-wise coupling yields ≧98% using the phosphoramidite procedures.

Purification of LNA oligomeric compounds was done using disposable reversed phase purification cartridges and/or reversed phase HPLC and/or precipitation from ethanol or butanol. Capillary gel electrophoresis, reversed phase HPLC, MALDI-MS, and ESI-MS was used to verify the purity of the synthesized oligonucleotides. Furthermore, solid support materials having immobilised thereto an optionally nucleobase protected and optionally 5′-OH protected LNA are especially interesting as material for the synthesis of LNA containing oligomeric compounds where an LNA monomer is included in at the 3′ end. In this instance, the solid support material is preferable CPG, e.g. a readily (commercially) available CPG material or polystyrene onto which a 3′-functionalised, optionally nucleobase protected and optionally 5′-OH protected LNA is linked using the conditions stated by the supplier for that particular material.

Indications

TRX is involved in a number of basic biological mechanisms including red blood cell proliferation, cellular proliferation, ion metabolism, glucose and energy metabolism, pH regulation and matrix metabolism. The methods of the invention is preferably employed for treatment or prophylaxis against diseases caused by cancer, particularly for treatment of cancer as may occur in tissue such as lung, breast, colon, prostate, pancreas, liver, brain, testes, stomach, intestine, bowel, spinal cord, sinuses, urinary tract or ovaries cancer.

The invention described herein encompasses a method of preventing or treating cancer comprising a therapeutically effective amount of a TRX modulating oligomeric compound, including but not limited to high doses of the oligomer, to a human in need of such therapy. The invention further encompasses the use of a short period of administration of a TRX modulating oligomeric compound. Normal, non-cancerous cells divide at a frequency characteristic for the particular cell type. When a cell has been transformed into a cancerous state, uncontrolled cell proliferation and reduced cell death results, and therefore, promiscuous cell division or cell growth is a hallmark of a cancerous cell type. Examples of types of cancer, include, but are not limited to, non-Hodgkin's lymphoma, Hodgkin's lymphoma, leukemia (e.g., acute leukemia such as acute lymphocytic leukemia, acute myelocytic leukemia, chronic myeloid leukemia, chronic lymphocytic leukemia, multiple myeloma), colon carcinoma, rectal carcinoma, pancreatic cancer, breast cancer, ovarian cancer, prostate cancer, renal cell carcinoma, hepatoma, bile duct carcinoma, choriocarcinoma, cervical cancer, testicular cancer, lung carcinoma, bladder carcinoma, melanoma, head and neck cancer, brain cancer, cancers of unknown primary site, neoplasms, cancers of the peripheral nervous system, cancers of the central nervous system, tumors (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, seminoma, embryonal carcinoma, Wilms' tumor, small cell lung carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, and retinoblastoma), heavy chain disease, metastases, or any disease or disorder characterized by uncontrolled or abnormal cell growth.

Pharmaceutical Composition

It should be understood that the invention also relates to a pharmaceutical composition, which comprises a least one antisense oligonucleotide construct of the invention as an active ingredient. It should be understood that the pharmaceutical composition according to the invention optionally comprises a pharmaceutical carrier, and that the pharmaceutical composition optionally comprises further antisense compounds, chemotherapeutic compounds, anti-inflammatory compounds, antiviral compounds and/or immuno-modulating compounds.

Salts

The oligomeric compound comprised in this invention can be employed in a variety of pharmaceutically acceptable salts. As used herein, the term refers to salts that retain the desired biological activity of the herein identified compounds and exhibit minimal undesired toxicological effects. Non-limiting examples of such salts can be formed with organic amino acid and base addition salts formed with metal cations such as zinc, calcium, bismuth, barium, magnesium, aluminum, copper, cobalt, nickel, cadmium, sodium, potassium, and the like, or with a cation formed from ammonia, N,N-dibenzylethylene-diamine, D-glucosamine, tetraethylammonium, or ethylenediamine; or (c) combinations of (a) and (b); e.g., a zinc tannate salt or the like.

Prodrugs

In one embodiment of the invention the oligomeric compound may be in the form of a pro-drug. Oligonucleotides are by virtue negatively charged ions. Due to the lipophilic nature of cell membranes the cellular uptake of oligonucleotides are reduced compared to neutral or lipophilic equivalents. This polarity “hindrance” can be avoided by using the pro-drug approach (see e.g. Crooke, R. M. (1998) in Crooke, S. T. Antisense research and Application. Springer-Verlag, Berlin, Germany, vol. 131, pp. 103-140). In this approach the oligonucleotides are prepared in a protected manner so that the oligo is neutral when it is administered. These protection groups are designed in such a way that so they can be removed then the oligo is taken up be the cells. Examples of such protection groups are S-acetylthioethyl (SATE) or S-pivaloylthioethyl (t-butyl-SATE). These protection groups are nuclease resistant and are selectively removed intracellulary.

Conjugates

In one embodiment of the invention the oligomeric compound is linked to ligands/conjugates. It is way to increase the cellular uptake of antisense oligonucleotides. This conjugation can take place at the terminal positions 5′/3′-OH but the ligands may also take place at the sugars and/or the bases. In particular, the growth factor to which the antisense oligonucleotide may be conjugated, may comprise transferrin or folate. Transferrin-polylysine-oligonucleotide complexes or folate-polylysine-oligonucleotide complexes may be prepared for uptake by cells expressing high levels of transferrin or folate receptor. Other examples of conjugates/lingands are cholesterol moieties, duplex intercalators such as acridine, poly-L-lysine, “end-capping” with one or more nuclease-resistant linkage groups such as phosphoromonothioate, and the like.

Formulations

The invention also includes the formulation of one or more oligonucleotide compound as disclosed herein. Pharmaceutically acceptable binding agents and adjuvants may comprise part of the formulated drug. Capsules, tablets and pills etc. may contain for example the following compounds: microcrystalline cellulose, gum or gelatin as binders; starch or lactose as excipients; stearates as lubricants; various sweetening or flavouring agents. For capsules the dosage unit may contain a liquid carrier like fatty oils. Likewise coatings of sugar or enteric agents may be part of the dosage unit. The oligonucleotide formulations may also be emulsions of the active pharmaceutical ingredients and a lipid forming a micellular emulsion.

An oligonucleotide of the invention may be mixed with any material that do not impair the desired action, or with material that supplement the desired action. These could include other drugs including other nucleoside compounds.

For parenteral, subcutaneous, intradermal or topical administration the formulation may include a sterile diluent, buffers, regulators of tonicity and antibacterials. The active compound may be prepared with carriers that protect against degradation or immediate elimination from the body, including implants or microcapsules with controlled release properties. For intravenous administration the preferred carriers are physiological saline or phosphate buffered saline.

Preferably, an oligomeric compound is included in a unit formulation such as in a pharmaceutically acceptable carrier or diluent in an amount sufficient to deliver to a patient a therapeutically effective amount without causing serious side effects in the treated patient.

Administration

The pharmaceutical compositions of the present invention may be administered in a number of ways depending upon whether local or systemic treatment is desired and upon the area to be treated. Administration may be (a) oral (b) pulmonary, e.g., by inhalation or insufflation of powders or aerosols, including by nebulizer; intratracheal, intranasal, (c) topical including epidermal, transdermal, ophthalmic and to mucous membranes including vaginal and rectal delivery; or (d) parenteral including intravenous, intraarterial, subcutaneous, intraperitoneal or intramuscular injection or infusion; or intracranial, e.g., intrathecal or intraventricular, administration. In one embodiment the active oligo is administered IV, IP, orally, topically or as a bolus injection or administered directly in to the target organ.

Pharmaceutical compositions and formulations for topical administration may include transdermal patches, ointments, lotions, creams, gels, drops, sprays, suppositories, liquids and powders. Conventional pharmaceutical carriers, aqueous, powder or oily bases, thickeners and the like may be necessary or desirable. Coated condoms, gloves and the like may also be useful. Preferred topical formulations include those in which the oligonucleotides of the invention are in admixture with a topical delivery agent such as lipids, liposomes, fatty acids, fatty acid esters, steroids, chelating agents and surfactants. Compositions and formulations for oral administration include but is not restricted to powders or granules, microparticulates, nanoparticulates, suspensions or solutions in water or non-aqueous media, capsules, gel capsules, sachets, tablets or minitablets. Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Delivery

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids. Delivery of drug to tumour tissue may be enhanced by carrier-mediated delivery including, but not limited to, cationic liposomes, cyclodextrins, porphyrin derivatives, branched chain dendrimers, polyethylenimine polymers, nanoparticles and microspheres (Dass C R. J Pharm Pharmacol 2002; 54(1):3-27).

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, gel capsules, liquid syrups, soft gels and suppositories. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

Combination Drug

Oligonucleotides of the invention may also be conjugated to active drug substances, for example, aspirin, ibuprofen, a sulfa drug, an antidiabetic, an antibacterial or an antibiotic.

LNA containing oligomeric compound are useful for a number of therapeutic applications as indicated above. In general, therapeutic methods of the invention include administration of a therapeutically effective amount of an LNA-modified oligonucleotide to a mammal, particularly a human.

In a certain embodiment, the present invention provides pharmaceutical compositions containing (a) one or more antisense compounds and (b) one or more other chemotherapeutic agents which function by a non-antisense mechanism. When used with the compounds of the invention, such chemotherapeutic agents may be used individually (e.g. mithramycin and oligonucleotide), sequentially (e.g. mithramycin and oligonucleotide for a period of time followed by another agent and oligonucleotide), or in combination with one or more other such chemotherapeutic agents or in combination with radiotherapy. All chemotherapeutic agents known to a person skilled in the art are here incorporated as combination treatments with compound according to the invention.

Anti-inflammatory drugs, including but not limited to nonsteroidal anti-inflammatory drugs and corticosteroids, antiviral drugs, and immuno-modulating drugs may also be combined in compositions of the invention. Two or more combined compounds may be used together or sequentially.

In another embodiment, compositions of the invention may contain one or more antisense compounds, particularly oligonucleotides, targeted to a first nucleic acid and one or more additional antisense compounds targeted to a second nucleic acid target. Two or more combined compounds may be used together or sequentially.

Dosage

Dosing is dependent on severity and responsiveness of the disease state to be treated, and the course of treatment lasting from several days to several months, or until a cure is effected or a diminution of the disease state is achieved. Optimal dosing schedules can be calculated from measurements of drug accumulation in the body of the patient.

Optimum dosages may vary depending on the relative potency of individual oligonucleotides. Generally it can be estimated based on EC50s found to be effective in in vitro and in vivo animal models. In general, dosage is from 0.01 μg to 1 g per kg of body weight, and may be given once or more daily, weekly, monthly or yearly, or even once every 2 to 10 years or by continuous infusion for hours up to several months. The repetition rates for dosing can be estimated based on measured residence times and concentrations of the drug in bodily fluids or tissues. Following successful treatment, it may be desirable to have the patient undergo maintenance therapy to prevent the recurrence of the disease state.

Uses

The LNA containing oligomeric compounds of the present invention can be utilized for as research reagents for diagnostics, therapeutics and prophylaxis. In research, the antisense oligonucleotides may be used to specifically inhibit the synthesis of TRX genes in cells and experimental animals thereby facilitating functional analysis of the target or an appraisal of its usefulness as a target for therapeutic intervention. In diagnostics the antisense oligonucleotides may be used to detect and quantitate TRX expression in cell and tissues by Northern blotting, in-situ hybridisation or similar techniques. For therapeutics, an animal or a human, suspected of having a disease or disorder, which can be treated by modulating the expression of TRX is treated by administering antisense compounds in accordance with this invention. Further provided are methods of treating an animal particular mouse and rat and treating a human, suspected of having or being prone to a disease or condition, associated with expression of TRX by administering a therapeutically or prophylactically effective amount of one or more of the antisense compounds or compositions of the invention.

### EXAMPLES

### Example 1

Monomer Synthesis

The LNA monomer building blocks and derivatives thereof were prepared following published procedures and references cited therein, see:

WO 03/095467 A1

D. S. Pedersen, C. Rosenbohm, T. Koch (2002) Preparation of LNA Phosphoramidites, Synthesis 6, 802-808.

M. D. Sørensen, L. Kværnø, T. Bryld, A. E. Håkansson, B. Verbeure, G. Gaubert, P. Herdewijn, J. Wengel (2002) α-L-ribo-configured Locked Nucleic Acid (α-l-LNA): Synthesis and Properties, J. Am. Chem. Soc., 124, 2164-2176.

S. K. Singh, R. Kumar, J. Wengel (1998) Synthesis of Novel Bicyclo[2.2.1] Ribonucleosides: 2′-Amino- and 2′-Thio-LNA Monomeric Nucleosides, J. Org. Chem. 1998, 63, 6078-6079.

Rosenbohm, S. M. Christensen, M. D. Sørensen, D. S. Pedersen, L. E. Larsen, J. Wengel, T. Koch (2003) Synthesis of 2′-amino-LNA: a new strategy, Org. Biomol. Chem. 1, 655-663.

Synthesis of the 2′-thio-LNA ribothymidine phosphoramidite. Reagents and conditions: i) Pd/C, H₂, acetone, MeOH; ii) BzCl, pyridine, DMF; iii) 0.25 M H₂SO₄ (aq), DMF, 80° C. (79% from 4; 3 steps); iv) Tf₂O, DMAP, CH₂Cl₂, 0° C.; v) Na₂S, DMF (72% from 7; 2 steps); vi) NaOBz, DMF, 100° C. (81%); vii) NH₃, MeOH (76%); viii) DMT-Cl, pyridine (88%); ix) P(OCH₂CH₂CN)(N(ⁱPr)₂)₂, 4,5-dicyanoimidazole, CH₂Cl₂ (99%). DMT=4,4′-dimethoxytrityl, PN₂=2-cyanoethoxy(diisopropylamino)phosphinoyl.

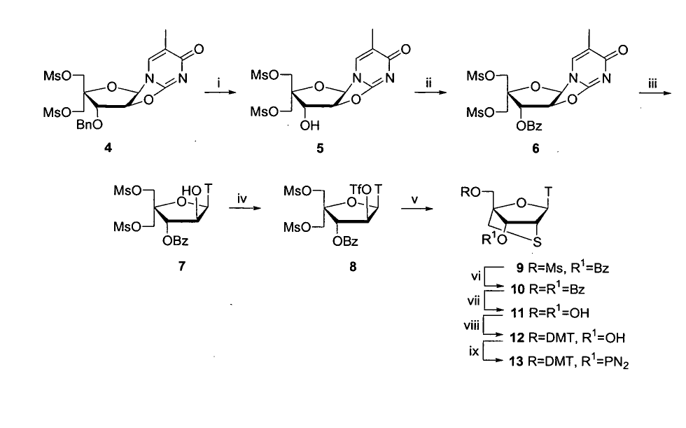

1-(3-O-Benzoyl-5-O-methanesulfonyl-4-C-methanesulfonyloxymethyl-□-D-threo-pentofuranosyl)thymine (7, FIG. 4)

Anhydro-nucleoside 4 (C. Rosenbohm, S. M. Christensen, M. D. Sørensen, D. S. Pedersen, L. E. Larsen, J. Wengel, T. Koch (2003) Synthesis of 2′-amino-LNA: a new strategy, Org. Biomol. Chem. 1, 655-663) (30.0 g, 58.1 mmol) was heated to 70° C. in a mixture of methanol (1000 cm³) and acetone (1000 cm³) until a clear solution was obtained and the solution was allowed to reach room temperature. The reaction flask was flushed with argon and Pd/C (10 wt. % Pd on carbon, 6.2 g, 5.8 mmol) was added. The mixture was stirred vigorously under an atmosphere of hydrogen gas (balloon). After 23 h the slurry was filtered through a pad of celite. The catalyst was recovered from the celite and refluxed in DMF (1000 cm³) for 1 h. The hot DMF slurry was filtered through a pad of celite and the organic layers combined and evaporated in vacuo to give nucleoside 5 as a yellow powder. Residual solvents were removed on a high vacuum pump overnight.

The crude nucleoside 5 (23 g) was heated to 70° C. in DMF (300 cm³) to give a clear yellow solution that was allowed to cool to room temperature. Benzoyl chloride (81.7 g, 581 mmol, 67.4 cm³) was added followed by pyridine (70 cm³). After 18 h the reaction was quenched with methanol (200 cm³) and excess methanol was removed in vacuo. To the dark brown solution of nucleoside 6 aqueous H₂SO₄ (0.25 M, 400 cm³) was added. The solution was heated to 80° C. on an oil bath (At approx 50° C. precipitation occurs. The solution becomes clear again at 80° C.). After 22 h at 80° C. the solution was allowed to cool to room temperature. The reaction mixture was transferred to a separatory funnel with ethyl acetate (1000 cm³). The organic layer was washed with sat. aq NaHCO₃ (2×1000 cm³). The combined aqueous layers were extracted with ethyl acetate (1000+500 cm³). The organic layers were combined and washed with sat. aq NaHCO₃ (1000 cm³), dried (Na₂SO₄), filtered and evaporated in vacuo to give a yellow liquid. Residual solvents were removed on a high vacuum pump overnight to give a yellow syrup. The product was purified by Dry Column Vacuum Chromatography (id 10 cm; 100 cm³ fractions; 50-100% EtOAc in n-heptane (v/v)—10% increments; 2-24% MeOH in EtOAc (v/v)—20% increments). Fractions containing the product were combined and evaporated in vacuo giving nucleoside 7 (25.1 g, 79%) as a white foam.

R_{f}=0.54 (5% MeOH in EtOAc, v/v);

ESI-MS m/z found 549.0 ([MH]⁺, calcd 549.1);

¹H NMR (DMSO-d₆) δ 11.39 (br s, 1H, NH), 8.10-8.08 (m, 2H, Ph), 7.74-7.70 (m, 1H, Ph), 7.60-7.56 (m, 2H, Ph), 7.51 (d, J=1.1 Hz, 1H, H6), 6.35 (d, J=4.9 Hz, 1H, H1′), 6.32 (d, J=5.3 Hz, 1H, 2′-OH), 5.61 (d, J=4.0 Hz, 1H, H3′), 4.69 (d, J=10.8 Hz, 1H), 4.59 (m, 1H, H2′), 4.55 (d, J=10.8 Hz, 1H), 4.52 (d, J=10.8 Hz, 1H), 4.46 (d, J=10.6 Hz, 1H) (H5′ and H1″), 3.28 (s, 3H, Ms), 3.23 (s, 3H, Ms), 1.81 (s, 3H, CH₃);

¹³C NMR (DMSO-d₆) δ 164.5, 163.6 (C4, PhC(O)), 150.3 (C2), 137.7 (C6), 133.8, 129.6, 128.7, 128.6 (Ph), 108.1 (C5), 84.8 (C1′), 81.1 (C4′), 78.0 (C3′), 73.2 (C2′), 68.0, 67.1 (C5′, C1″), 36.7, 36.6 (2×Ms), 11.9 (CH₃);

Elemental anal. calcd for C₂₀H₂₄N₂O₁₂S₂.0.33 H₂O (%): C, 44.34; H, 4.65; N, 4.85. Found: C, 44.32; H, 4.58; N, 4.77.

(1R,3R,4R,7R)-7-Benzoyloxy-1-methansulfonyloxymethyl-3-(thymin-1-yl)-2-oxa-5-thiabicyclo[2:2:1]heptane (9)

1-(3-O-Benzoyl-5-O-methanesulfonyl-4-C-methanesulfonyloxymethyl-□-D-threo-pentofuranosyl)thymine (7) (10.00 g, 18.23 mmol) was dissolved in dichloromethane (500 cm³) and cooled to 0° C. Pyridine (15 cm³) and DMAP (8.91 g, 72.9 mmol) was added followed by dropwise addition of trifluoromethanesulfonic anhydride (10.30 g, 36.5 mmol, 6.0 cm³). After 1 h the reaction was quenched with sat. aq NaHCO₃ (500 cm³) and transferred to a separatory funnel. The organic layer was washed with 1.0 M aq HCl (500 cm³), sat. aq NaHCO₃ (500 cm³) and brine (500 cm³). The organic layer was evaporated in vacuo with toluene (100 cm³) to give 1-(3-O-benzoyl-5-O-methanesulfonyl-4-C-methanesulfonyloxymethyl-2-O-trifluoromethanesulfonyl-β-D-threo-pentofuranosyl)thymine (8) as a yellow powder.

The crude nucleoside 8 was dissolved in DMF (250 cm³) and Na₂S (1.57 g, 20.1 mmol) was added to give a dark green slurry. After 3 h the reaction was quenched with half sat. aq NaHCO₃ (500 cm³) and extracted with dichloromethane (500+2×250 cm³). The combined organic layers were washed with brine (500 cm³), dried (Na₂SO₄), filtered and concentrated in vacuo to give a yellow liquid. Residual solvent was removed overnight on a high vacuum pump to give a yellow gum that was purified by Dry Column Vacuum Chromatography (id 6 cm: 50 cm³ fractions; 50-100% EtOAc in n-heptane (v/v)—10% increments; 2-20% MeOH in EtOAc (v/v)—2% increments) to give nucleoside 9 (6.15 g, 72%) as a yellow foam.

R_{f}=0.27 (20% n-heptane in EtOAc, v/v);

ESI-MS m/z found 469.0 ([MH]⁺, calcd 469.1);

¹H NMR (CDCl₃) δ 8.70 (br s, 1H, NH), 8.01-7.99 (m, 2H, Ph), 7.67 (d, J=1.1 Hz, 1H, H6), 7.65-7.61 (m, 1H, Ph), 7.50-7.46 (m, 2H, Ph), 5.98 (s, 1H, H1′), 5.34 (d, J=2.4 Hz, 1H, H3′), 4.66 (d, J=11.7 Hz, 1H, H5′a), 4.53 (d, J=11.5 Hz, 1H, H5′b), 4.12 (m (overlapping with residual EtOAc), 1H, H2′), 3.15-3.13 (m, 4H, H1″a and Ms), 3.06 (d, J=10.6 Hz, 1H, H1″b), 1.98 (d, J=1.1 Hz, 3H, CH₃);

¹³C NMR (CDCl₃) δ 165.2, 163.5 (C4, PhC(O)), 149.9 (C2), 134.1, 133.9, 129.8, 128.7, 128.3 (C6, Ph), 110.7 (C5), 91.1 (C1′), 86.8 (C4′), 72.6 (C3′), 65.8 (C5′), 50.5 (C2′), 37.9 (Ms), 35.1 (C1″), 12.5 (CH₃);

Elemental anal. calcd for C₁₉H₂₀N₂O₈S₂.0.33 EtOAc (%): C, 49.21; H, 4.72; N, 5.47.

Found: C, 49.25; H, 4.64; N, 5.48.

(1R,3R,4R,7R)-7-Benzoyloxy-1-benzoyloxymethyl-3-(thymin-1-yl)-2-oxa-5-thiabicyclo[2:2:1]heptane (10)

Nucleoside 9 (1.92 g, 4.1 mmol) was dissolved in DMF (110 cm³). Sodium benzoate (1.2 g, 8.2 mmol) was added and the mixture was heated to 100° C. for 24 h. The reaction mixture was transferred to a separatory funnel with half sat. brine (200 cm³) and extracted with ethyl acetate (3×100 cm³). The combined organic layers were dried (Na₂SO₄), filtered and evaporated in vacuo to give a brown liquid. The product was put on a high vacuum pump to remove residual solvent. The resulting brown gum was purified by Dry Column Vacuum Chromatography (id 4 cm; 50 cm³ fractions; 0-100% EtOAc in n-heptane (v/v)—10% increments; 2-10% MeOH in EtOAc (v/v) −2% increments) to give nucleoside 10 (1.64 g, 81%) as a slightly yellow foam.

R_{f}=0.57 (20% n-heptane in EtOAc, v/v);

ESI-MS m/z found 495.1 ([MH]⁺, calcd 495.1);

¹H NMR (CDCl₃) δ 9.02 (br s, 1H, NH), 8.07-7.99 (m, 4H, Ph), 7.62-7.58 (m, 2H, Ph), 7.47-7.42 (m, 5H, Ph and H6), 5.95 (s, 1H, H1′), 5.46 (d, J=2.2 Hz, 1H, H3′), 4.93 (d, J=12.8 Hz, 1H, H5′a), 4.60 (d, J=12.8 Hz, 1H, H5′b), 4.17 (d, J=2.2 Hz, 1H, H2′), 3.27 (d, J=10.6 Hz, 1H, H1″a), 3.16 (d, J=10.6 Hz, 1H, H1″b), 1.55 (d, J=1.1 Hz, 3H, CH₃);

¹³C NMR (CDCl₃) δ 165.8, 165.1, 163.7 (C4, 2×PhC(O)), 150.0 (C2), 133.9, 133.7, 133.6, 129.8, 129.6, 129.0, 128.8, 128.6, 128.5 (C6, 2×Ph), 110.3 (C5), 91.3 (C1′), 87.5 (C4′), 72.9 (C3′), 61.3 (C5′), 50.6 (C2′), 35.6 (C1″), 12.3 (CH₃);

Elemental anal. calcd for C₂₅H₂₂N₂O₇S (%): C, 60.72; H, 4.48; N, 5.66. Found: C, 60.34; H, 4.49; N, 5.35.

(1R,3R,4R,7R)-7-Hydroxy-1-hydroxymethyl-3-(thymin-1-yl)-2-oxa-5-thiabicyclo[2:2:1]heptane (11)

Nucleoside 10 (1.50 g, 3.0 mmol) was dissolved in methanol saturated with ammonia (50 cm³). The reaction flask was sealed and stirred at ambient temperature for 20 h. The reaction mixture was concentrated in vacuo to give a yellow gum that was purified by Dry Column Vacuum Chromatography (id 4 cm; 50 cm³ fractions; 0-16% MeOH in EtOAc (v/v)—1% increments) giving nucleoside 11 (0.65 9, 76%) as clear needles.

R_{f}=0.31 (10% MeOH in EtOAc, v/v);

ESI-MS m/z found 287.1 ([MH]⁺, calcd 287.1);

¹H NMR (DMSO-d₆) δ 11.32 (br s, 1H, NH), 7.96 (d, J=1.1 Hz, 1H, H6), 5.95 (s, 1H), H6), 5.70 (d, J=4.2 Hz, 1H, 3′-OH), 5.62 (s, 1H, H1′), 4.49 (t, J=5.3 Hz, 1H, 5′-OH), 4.20 (dd, J=4.1 and 2.1 Hz, 1H, H3′), 3.77-3.67 (m, 2H, H5′), 3.42 (d, J=2.0 Hz, 1H, H2′), 2.83 (d, J=10.1 Hz, 1H, H1″a), 2.64 (d, J=10.1 Hz, 1H, H1″b), 1.75 (d, J=1.1 Hz, 3H, CH₃);

¹³C NMR (DMSO-d₆) δ 163.8 (C4), 150.0 (C2), 135.3 (C6), 107.5 (C5), 90.2, 89.6 (C1′and C4′), 69.4 (C3′), 58.0 (C5′), 52.1 (C2′), 34.6 (C1″), 12.4 (CH₃);

Elemental anal. calcd for C₁₁H₁₄N₂O₅S (%): C, 46.15; H, 4.93; N, 9.78. Found: C, 46.35; H, 4.91; N, 9.54.

(1R,3R,4R,7R)-1-(4,4′-Dimethoxytrityloxymethyl)-7-hydroxy-5-methyl-3-(thymin-1-yl)-2-oxa-5-thiabicyclo[2:2:1]heptane (12)

Nucleoside 11 (0.60 g, 2.1 mmol) was dissolved in pyridine (10 cm³). 4,4′-Dimethoxytrityl chloride (0.88 g, 2.6 mmol) was added and the reaction was stirred at ambient temperature for 3 h. The reaction mixture was transferred to a separatory funnel with water (100 cm³) and extracted with ethyl acetate (100+2×50 cm³). The combined organic layers were washed with sat. aq NaHCO₃ (100 cm³), brine (100 cm³) and evaporated to dryness in vacuo to give a viscous yellow liquid. The product was redissolved in toluene (50 cm³) and concentrated in vacuo to give a yellow foam. The foam was dried on a high vacuum pump overnight and purified by Dry Column Vacuum Chromatography (id 4 cm; 50 cm³ fractions; 10-100% EtOAc in n-heptane (v/v)—10% increments) giving nucleoside 12 (1.08 g, 88%) as a white foam.

R_{f}=0.24 (20% n-heptane in EtOAc, v/v);

ESI-MS m/z found 587.1 ([M−H]⁺, calcd 587.2);

¹H NMR (CDCl₃) δ 8.96 (br s, 1H, NH), 7.74 (d, J=1.1 Hz, 1H, H6), 7.46-7.44 (m, 2H, Ph), 7.35-7.22 (m, 9H, Ph), 7.19-7.15 (m, 2H, Ph), 6.86-6.80 (m, 2H, Ph), 5.82 (s, 1H, H1′), 4.55 (dd, J=9.3 and 2.1 Hz, 1H, H3′), 3.79 (s, 6H, OCH₃), 3.71 (d, J=2.0 Hz, 1H, H2′), 3.50 (s, 2H, H5′), 2.81 (d, J=10.8 Hz, 1H, H1″a), 2.77 (d, J=10.8 Hz, 1H, H1″b), 2.69 (d, J=9.2 Hz, 1H, 3′-OH), 1.42 (s, 3H, CH₃);

¹³C NMR (CDCl₃) δ 158.7 (C4), 150.1 (C2), 144.1, 135.2, 135.1, 130.1, 129.1, 128.1, 128.0, 127.1, 127.0, 113.3 (C6, 3×Ph), 110.0 (C5), 90.2 (C(Ph)₃), 89.6 (C1′), 87.0 (C4′), 71.7 (C3′), 60.9 (C5′), 55.2 (C2′), 34.7 (C1″), 12.2 (CH₃);

Elemental anal. calcd for C₃₂H₃₂N₂O₇S.0.5 H₂O (%): C, 64.31; H, 5.57; N, 4.69. Found: C, 64.22; H, 5.67; N, 4.47.

(1R,3R,4R,7R)-7-(2-Cyanoethoxy(diisopropylamino)phosphinoxy)-1-(4,4′-dimethoxytrityloxymethyl)-3-(thymin-1-yl)-2-oxa-5-thiabicyclo[2.2.1]heptane (13)

According to the published method (D. S. Pedersen, C. Rosenbohm, T. Koch (2002) Preparation of LNA Phosphoramidites, Synthesis, 6, 802-808) nucleoside 12 (0.78 g, 1.33 mmol) was dissolved in dichloromethane (5 cm³) and a 1.0 M solution of 4,5-dicyanoimidazole in acetonitrile (0.93 cm³, 0.93 mmol) was added followed by dropwise addition of 2-cyanoethyl-N,N,N′,N′-tetraisopropylphosphorodiamidite (0.44 cm³, 1.33 mmol). After 2 h the reaction was transferred to a separatory funnel with dichloromethane (40 cm³) and washed with sat. aq NaHCO₃ (2×25 cm³) and brine (25 cm³). The organic layer was dried (Na₂SO₄), filtered and evaporated in vacuo to give nucleoside 13 (1.04 g, 99%) as a white foam. R_{f}=0.29 and 0.37-two diastereoisomers (20% n-heptane in EtOAc, v/v); ESI-MS m/z found 789.3 ([MH]⁺, calcd 789.3); ³¹P NMR (DMSO-d₆) δ 150.39, 150.26.

### Example 2

Oligonucleotide Synthesis

Oligonucleotides were synthesized using the phosphoramidite approach on an Expedite 8900/MOSS synthesizer (Multiple Oligonucleotide Synthesis System) at 1 or at 15 μmol. At the end of the synthesis (DMT-on) the oligonucleotides were cleaved from the solid support using aqueous ammonia for 1 h at room temperature, and further deprotected for 3 h at 65° C. The oligonucleotides were purified by reverse phase HPLC (RP-HPLC). After the removal of the DMT-group, the oligonucleotides were characterized by IE-HPLC or RP-HPLC. The identity of the oligonucleotides is confirmed by ESI-MS. See below for more details.

Preparation of the LNA Succinyl Hemiester

5′-O-Dmt-3′-hydroxy-LNA monomer (500 mg), succinic anhydride (1.2 eq.) and DMAP (1.2 eq.) were dissolved in DCM (35 mL). The reaction was stirred at room temperature overnight. After extractions with NaH₂PO₄ 0.1 M pH 5.5 (2×) and brine (1×), the organic layer was further dried with anhydrous Na₂SO₄ filtered and evaporated. The hemiester derivative was obtained in 95% yield and was used without any further purification.

Preparation of the LNA-support

The above prepared hemiester derivative (90 μmol) was dissolved in a minimum amount of DMF, DIEA and pyBOP (90 μmol) were added and mixed together for 1 min. This pre-activated mixture was combined with LCAA-CPG (500 Å, 80-120 mesh size, 300 mg) in a manual synthesizer and stirred. After 1.5 h at room temperature, the support was filtered off and washed with DMF, DCM and MeOH. After drying the loading was determined to be 57 μmol/g (see Tom Brown, Dorcas J. S. Brown, “Modern machine-aided methods of oligodeoxyribonucleotide synthesis”, in: F.Eckstein, editor. Oligonucleotides and Analogues A Practical Approach. Oxford: IRL Press, 1991: 13-14).

Elongation of the Oligonucleotide

The coupling of phosphoramidites (A(bz), G(ibu), 5-methyl-C(bz)) or T-β-cyanoethyl-phosphoramidite) is performed by using a solution of 0.1 M of the 5′-O-DMT-protected amidite in acetonitrile and DCI (4,5-dicyanoimidazole) in acetonitrile (0.25 M) as activator. The thiolation is carried out by using xanthane chloride (0.01 M in acetonitrile:pyridine 10%). The rest of the reagents are the ones typically used for oligonucleotide synthesis.

Purification by RP-HPLC:

| | |
|---|---|
| Column: | XTerra, RP18, 5 μm, 7.8 × 50 mm column. |
| Eluent: | Eluent A: 0.1 M NH₄OAc, pH: 10. |
| | Eluent B: Acetonitrile |
| Flow: | 5 ml/min. |

Gradient:

| Time (min.) | Eluent A | Eluent B |
|---|---|---|
| 0.05 min. | 95% | 5% |
| 5 min. | 95% | 5% |
| 12 min. | 65% | 35% |
| 16 min. | 0% | 100% |
| 19 min. | 0% | 100% |
| 21 min | 100% | 0% |

Analysis by IE-HPLC:

| | |
|---|---|
| Column: | Dionex, DNAPac PA-100, 2 × 250 mm column. |
| Eluent: | Eluent A: 20 mM Tris-HCl, pH 7.6; 1 mM EDTA; |
| | 10 mM NaClO₄. |
| | Eluent B: 20 mM Tris-HCl, pH 7.6; 1 mM EDTA; |
| | 1 M NaClO₄. |
| Flow: | 0.25 ml/min. |

Gradient:

| Time (min.) | Eluent A | Eluent B |
|---|---|---|
| 1 min. | 95% | 5% |
| 10 min. | 65% | 35% |
| 11 min. | 0% | 100% |
| 15 min. | 0% | 100% |
| 16 min | 95% | 5% |
| 21 min. | 95% | 5% |

Abbreviations

| | |
|---|---|
| DMT: | Dimethoxytrityl |
| DCI: | 4,5-Dicyanoimidazole |
| DMAP: | 4-Dimethylaminopyridine |
| DCM: | Dichloromethane |
| DMF: | Dimethylformamide |
| THF: | Tetrahydrofurane |
| DIEA: | N,N-diisopropylethylamine |
| PyBOP: | Benzotriazole-1-yl-oxy-tris-pyrrolidino-phosphonium |
| | hexafluorophosphate |
| Bz: | Benzoyl |
| Ibu: | Isobutyryl |

### Example 3

Test of Design of the Oligomeric Compound

It was of our interest to evaluate the antisense activity of oligonucleotides with different designs, in order to prove the importance of choosing the best design for an oligonucleotide targeting TRX. For this purpose, we set up an in vitro assay that would allow us to screen many different oligonucleotide designs by measuring the activity of the firefly (Photinus pyralis) luciferase after down-regulation by antisense oligonucleotides. FIG. 1 contains an illustration of the designs mentioned in the text.

In a first screen, designs containing β-D-oxy-LNA, which were all targeting the same motif within the mRNA were evaluated. Designs consisting of gapmers with a different gap-size, a different load of phosphorothioate internucleoside linkages, and a different load of LNA were tested. Headmers and tailmers with a different load of β-D-oxy-LNA, a different load of phosphorothioate internucleoside linkages and a different load of DNA were prepared. Mixmers of various compositions, which means that bear an alternate number of units of β-D-oxy-LNA, α-L-LNA and DNA, were also analysed in the in vitro assay. Moreover, LNA derivatives were also included in different designs, and their antisense activity was assessed. The importance of a good design is reflected by the data that can be obtained in a luciferase assay. The luciferase expression levels are measured in %, and give an indication of the antisense activity of the different designs containing β-D-oxy-LNA and LNA derivatives. We can easily see that some designs are potent antisense oligonucleotides, while others give moderate to low down-regulation levels. Therefore, a close correlation between good antisense activity and optimal design of an oligonucleotide is very evident. We appreciated good levels of down-regulation with various designs. Gapmers with gaps of 7-10 nt DNA and thiolation all over the backbone or with thiolation exclusively in the gap and PO in the flanks showed good results. These designs contain β-D-oxy-LNA or LNA derivatives. Headmers of 6 nt and 8 nt β-D-oxy-LNA also presented good levels of down-regulation, when the phosphorothioate internucleoside linkages are all over the backbone or only in the DNA-segment. Different mixmers gave good antisense activity in the luciferase assay. The alternate number of units of each α-L-oxy-LNA, β-D-oxy-LNA or DNA composition defines the mixmers, see FIG. 1. A mixmer 3-9-3-1, which has a deoxynucleoside residue at the 3′-end showed significant levels of down-regulation. In a mixmer 4-1-1-5-1-1-3, we placed two α-L-oxy-LNA residues interrupting the gap, being the flanks β-D-oxy-LNA. Furthermore, we interrupted the gap with two α-L-oxy-LNA residues, and substituted both flanks with α-L-oxy-LNA. Both designs presented significant levels of down-regulation. The presence of α-L-oxy-LNA might introduce a flexible transition between the North-locked flanks (oxy-LNA) and the α-L-oxy-LNA residue by spiking in deoxynucleotide residues. It is also interesting to study design 4-3-1-3-5 where a α-L-oxy-LNA residue interrupts the DNA stretch. In addition to the α-L-oxy-LNA in the gap, we also substituted two oxy-LNA residues at the edges of the flanks with two α-L-oxy-LNA residues. The presence of just one β-D-oxy-LNA residue (design 4-3-1-3-5) interrupting the stretch of DNAs in the gap results in a dramatic loss of down-regulation. Just by using α-L-oxy-LNA instead, the design shows significant down-regulation at 50 nM oligonucleotide concentration. The placement of α-L-oxy-LNA in the junctions and one α-L-oxy-LNA in the middle of the gap also showed down-regulation.

α-L-oxy-LNA reveals to be a potent tool enabling the construction of different mixmers, which are able to present high levels of antisense activity. Other mixmers such as 4-1-5-1-5 and 3-3-3-3-3-1 can also be prepared. We can easily see that some designs are potent antisense oligonucleotides, while others give moderate to low down-regulation levels. Therefore, again a close correlation between good antisense activity and optimal design of an oligonucleotide is very evident. Other preferred designs are (1-3-8-3-1) where DNA residues are located in the flanks with 3 β-D-oxy-LNA monomers at each side of the gap. A further preferred design is (4-9-3-1) with D-oxy-LNA flanks and a 9 gap with a DNA at the 3′-end.

### Example 4

In vitro Model: Cell Culture

The effect of antisense compounds on target nucleic acid expression can be tested in any of a variety of cell types provided that the target nucleic acid is present at measurable levels. Target can be expressed endogenously or by transient or stable transfection of a nucleic acid encoding said nucleic acid. The expression level of target nucleic acid can be routinely determined using, for example, Northern blot analysis, Real-Time PCR, Ribonuclease protection assays. The following cell types are provided for illustrative purposes, but other cell types can be routinely used, provided that the target is expressed in the cell type chosen.

Cells were cultured in the appropriate medium as described below and maintained at 37° C. at 95-98% humidity and 5% CO₂. Cells were routinely passaged 2-3 times weekly.

15PC3: The human prostate cancer cell line 15PC3 was kindly donated by Dr. F. Baas, Neurozintuigen Laboratory, AMC, The Netherlands and was cultured in DMEM (Sigma)+10% fetal bovine serum (FBS)+Glutamax I+gentamicin

A549: The human non-small cell lung cancer cell line A549 was purchased from ATCC, Manassee and was cultured in DMEM (Sigma)+10% FBS+Glutamax I+gentamicin

MCF7: The human breast cancer cell line MCF7 was purchased from ATCC and was cultured in Eagle MEM (Sigma)+10% FBS+Glutamax I+gentamicin

SW480: The human colon cancer cell line SW480 was purchased from ATCC and was cultured in L-15 Leibovitz (Sigma)+10% FBS+Glutamax I+gentamicin

SW620: The human colon cancer cell line SW620 was purchased from ATCC and was cultured in L-15 Leibovitz (Sigma)+10% FBS+Glutamax I+gentamicin

HT29: The human prostate cancer cell line HT29 was purchased from ATCC and was cultured in McCoy's 5a MM (Sigma)+10% FBS+Glutamax I+gentamicin

NCI H23: The human non-small-cell lung cancer cell line was purchased from ATCC and was cultured in RPMI 1640 with Glutamax I (Gibco)+10% FBS+HEPES+gentamicin

HCT-116: The human colon cancer cell line HCT-116 was purchased from ATCC and was cultured in McCoy's 5a MM (Sigma)+10% FBS+Glutamax I+gentamicin

MDA-MB-231: The human breast cancer cell line MDA-MB-231 was purchased from ATCC and was cultured in L-15 Leibovitz (Sigma)+10% FBS+Glutamax I+gentamicin

MDA-MB-435s: The human breast cancer cell line MDA-MB-435s was purchased from ATCC and was cultured in L-15 Leibovitz (Sigma)+10% FBS+Glutamax I+gentamicin

DMS273: The human small-cell lung cancer cell line DMS273 was purchased from ATCC and was cultured in Waymouth with glutamine (Gibco)+10% FBS+gentamicin

PC3: The human prostate cancer cell line PC3 was purchased from ATCC and was cultured in F12 Coon's with glutamine (Gibco)+7% FBS+gentamicin

U373: The human glioblastoma astrocytoma cancer cell line U373 was purchased from ECACC and was cultured in EMEM+10% FBS+glutamax+NEAA+sodiumpyrovate+gentamicin.

HUVEC: The human umbilical vein endothelial cell line was purchased from ATCC. HUVEC-C human umbilical vein endothelial cells were purchased from ATCC and propagated according to the manufacturers instructions.

HMVEC-d (DMVEC's-dermal human microvascular endothelial cells) were purchased from Clonetics and cultured as described by manufacturer.

HMVEC human microvascular endothelial cells were purchased from Clonetics and cultured as stated by manufacturer

Human embryonic lung fibroblasts were purchased from ATCC and cultured as described by manufacturer

HMEC-1 Human mammary epithelial cells were purchased from Clonetics and maintained as recommended by the manufacturer

### Example 5

In vitro Model: Treatment with Antisense Oligonucleotide

The cells were treated with oligonucleotide using the cationic liposome formulation LipofectAMINE 2000 (Gibco) as transfection vehicle.

Cells were seeded in 12-well cell culture plates (NUNC) and treated when 80-90% confluent. Oligo concentrations used ranged from 125 nM to 0,2 nM final concentration. Formulation of oligo-lipid complexes were carried out essentially as described in Dean et al. (Journal of Biological Chemistry 1994, 269, 16416-16424) using serum-free OptiMEM (Gibco) and a final lipid concentration of 10 μg/mlLipofectAMINE 2000 in 500 μl total volume.

Cells were incubated at 37° C. for 4 hours and treatment was stopped by removal of oligo-containing culture medium. Cells were washed and serum-containing media was added. After oligo treatment cells were allowed to recover for 18 hours (otherwise as stated in the figure legends) before they were harvested for RNA or protein analysis.

### Example 6

In vitro Model: Extraction of RNA and cDNA Synthesis

Total RNA Isolation

Total RNA was isolated either using RNeasy mini kit (Qiagen cat. no. 74104) or using the Trizol reagent (Life technologies cat. no. 15596). For RNA isolation from cell lines, RNeasy is the preferred method and for tissue samples Trizol is the preferred method. Total RNA was isolated from cell lines using the Qiagen RNA OPF Robot—BIO Robot 3000 according to the protocol provided by the manufacturer. Tissue samples were homogenised using an Ultra Turrax T8 homogeniser (IKA Analysen technik) and total RNA was isolated using the Trizol reagent protocol provided by the manufacturer.

First Strand Synthesis

First strand synthesis was performed using OmniScript Reverse Transcriptase kit (cat# 205113, Qiagen) according to the manufacturers instructions. For each sample 0.5 μg total RNA was adjusted to 12 μl each with RNase free H₂O and mixed with 2 μl poly (dT)₁₂₋₁₈ (2.5 μg/ml) (Life Technologies, GibcoBRL, Roskilde, DK), 2 μl dNTP mix (5 mM each dNTP), 2 μl 10× Buffer RT, 1 μl RNAguard™Rnase INHIBITOR (33.3 U/ml), (cat# 27-0816-01, Amersham Pharmacia Biotech, Hørsholm, DK) and 1 μl OmniScript Reverse Transcriptase (4 U/μl) followed by incubation at 37° C. for 60 minutes and heat inactivation of the enzyme at 93° C. for 5 minutes.

### Example 7

In vitro Model: Analysis of Oligonucleotide Inhibition of TRX Expression by Real-time PCR

Antisense modulation of TRX expression can be assayed in a variety of ways known in the art. For example, TRX mRNA levels can be quantitated by, e.g., Northern blot analysis, competitive polymerase chain reaction (PCR), or real-time PCR. Real-time quantitative PCR is presently preferred. RNA analysis can be performed on total cellular RNA or mRNA.

Methods of RNA isolation and RNA analysis such as Northern blot analysis is routine in the art and is taught in, for example, Current Protocols in Molecular Biology, John Wiley and Sons.

Real-time quantitative (PCR) can be conveniently accomplished using the commercially iQ Multi-Color Real Time PCR Detection System available from BioRAD.

Real-time Quantitative PCR Analysis of TRX mRNA Levels

Quantitation of mRNA levels was determined by real-time quantitative PCR using the iQ Multi-Color Real Time PCR Detection System (BioRAD) according to the manufacturers instructions. Real-time Quantitative PCR is a technique well known in the art and is taught in for example Heid et al. Real time quantitative PCR, Genome Research (1996), 6: 986-994.

Platinum Quantitative PCR SuperMix UDG 2× PCR master mix was obtained from Invitrogen cat# 11730. Primers and TaqMan® probes were obtained from MWG-Biotech AG, Ebersberg, Germany

Probes and primers to human TRX were designed to hybridise to a human TRX sequence, using published sequence information (GenBank accession number NM 003329, incorporated herein as SEQ ID NO:1).

For human TRX the PCR primers were:

forward primer: 5′ aagcctttctttcattccctctc 3′ (final concentration in the assay; 0.3 μM) reverse primer: 5′ cttcttaaaaaactggaatgttggc 3′ (final concentration in the assay; 0.3 μM) (SEQ ID NO: 59) and the PCR probe was: 5′ FAM-gatgtggatgactgtcaggatgttgcttc-TAMRA 3′ (final concentration in the assay; 0.1 μM)

Glyceraldehyde-3-phosphate dehydrogenase (GAPDH) mRNA quantity was used as an endogenous control for normalizing any variance in sample preparation.

The sample content of human GAPDH mRNA was quantified using the human GAPDH ABI Prism Pre-Developed TaqMan Assay Reagent (Applied Biosystems cat. no. 4310884E) according to the manufacturers instructions.

For quantification of mouse GAPDH mRNA the following primers and probes were designed: Sense primer 5′aaggctgtgggcaaggtcatc 3′ (0.3 μM final concentration), antisense primer 5′ gtcagatccacgacggacacatt (0.6 μM final concentration), TaqMan probe 5′ FAM-gaagctcactggcatggcatggccttccgtgtt c-TAMRA 3′ (0.2 μM final concentration).

Real Time PCR

The cDNA from the first strand synthesis performed as described in example 8 was diluted 2-20 times, and analyzed by real time quantitative PCR. The primers and probe were mixed with 2× Platinum Quantitative PCR SuperMix UDG (cat. # 11730, Invitrogen) and added to 3.3 μl cDNA to a final volume of 25 μl. Each sample was analysed in triplicates. Assaying 2 fold dilutions of a cDNA that had been prepared on material purified from a cell line expressing the RNA of interest generated standard curves for the assays. Sterile H₂O was used instead of cDNA for the no template control. PCR program: 50° C. for 2 minutes, 95° C. for 10 minutes followed by 40 cycles of 95° C., 15 seconds, 60° C., 1 minutes.

Relative quantities of target mRNA sequence were determined from the calculated Threshold cycle using the icycler iQ Real-time Detection System software.

### Example 8

In vitro Analysis: Northern Blot Analysis of TRX mRNA Levels

Northern blot analysis was carried out by procedures well known in the art essentially as described in Current Protocols in Molecular Biology, John Wiley & Sons.

The hybridisation probe was obtained by PCR-amplification of a TRX bp fragment from TRX cDNA obtained by reverse transcription PCR as described in example 8. The reaction was carried out using primers 5′ ggatccatttccatcggtcc 3′ (forward) and 5′ gcagatggcaactggttatgtct 3′ (reverse) at 0,5 μM final concentration each, 200 nM each dNTP, 1,5 mM MgCl₂ and Platinum Taq DNA polymerase (Invitrogen cat. no. 10966-018). The DNA was amplified for 40 cycles on a Perkin Elmer 9700 thermocycler using the following program: 94° C. for 2 min. then 40 cycles of 94° C. for 30 sec. and 72° C. for 30 sec. with a decrease of 0.5° C. per cycle followed by 72° C. for 7 min.

The amplified PCR product was purified using S-400 MicroSpin columns (Amersham Pharmacia Biotech cat. no. 27-5140-01) according to the manufacturers instructions and quantified by spectrophotometry.

The hybridisation probe was labelled using Redivue™ [α-³²P]dCTP 3000 Ci/mmol (Amersham Pharmacia Biotech cat. no. AA 0005) and Prime-It RmT labeling kit (Stratagene cat. no. 300392) according to the manufacturers instructions and the radioactively labeled probe was purified using S-300 MicroSpin columns (Amersham Pharmacia Biotech cat. no. 27-5130-01). Before use, the probe was denatured at 96° C. and immediately put on ice.

Samples of 1-5 μg of total RNA purified as described in example 7 were denatured and size separated on a 2,2 M formaldehyde/MOPS agarose gel.

RNA was transferred to positively charged nylon membrane by downward capillary transfer using the TurboBlotter (Schleicher & Schuell) and the RNA was immobilised to the membrane by UV crosslinking using a Stratagene crosslinker.

The membrane was prehybridised in ExpressHyb Hybridization Solution (Clontech cat. No. 8015-1) at 60° C. and the probe was subsequently added for hybridisation. Hybridisation was carried out at 60° C. and the blot was washed with low stringency wash buffer (2×SSC, 0,1% SDS) at room temperature and with high stringency wash buffer (0,1×SSC, 0,1% SDS) at 50° C.

The blot was exposed to Kodak storage phosphor screens and scanned in a BioRAD FX molecular imager. TRX mRNA levels were quantified by Quantity One software (BioRAD) Equality of RNA sample loading was assessed by stripping the blot in 0,5% SDS in H₂O at 85° C. and reprobing with a labelled GAPDH (glyceraldehyde-3-phosphate dehydrogenase) probe obtained essentially as described above using the primers 5′ aac gga ttt ggt cgt att 3′ (forward) and 5′ taa gca gtt ggt ggt gca 3′ (reverse).

FIG. 2 and 3 show TRX inhibition that were normalised to GAPDH. Intensity was monitored with phosphoimager Biorad, FX-scanner (see table 1). The tested oligomeric compounds are presented in Example 10.

TABLE 1
Percentage down regulation of mRNA estimated from
Trx Northern blotting (data is normalised to GAPDH).

| | Seq ID | | | | |
|---|---|---|---|---|---|
| Compound | 0 nM | 0.2 nM | 1 nM | 5 nM | 25 nM |
| Cur2675 | | 20% | 72% | 84% | 88% |
| Cur2676 | | 20% | 50% | 72% | 84% |
| Cur2677 | | 21% | 65% | 72% | 82% |
| Cur2681 | | 13% | 43% | 65% | 89% |
| Mock | 100% | | | | |

### Example 9

In vitro Analysis: Western Blot Analysis of TRX Protein Levels

Protein levels of TRX can be quantitated in a variety of ways well known in the art, such as immunoprecipitation, Western blot analysis (immunoblotting), ELISA, RIA (Radio Immuno Assay) or fluorescence-activated cell sorting (FACS). Antibodies directed to TRX can be identified and obtained from a variety of sources, such as Upstate Biotechnologies (Lake Placid, USA), Novus Biologicals (Littleton, Colo.), Santa Cruz Biotechnology (Santa Cruz, Calif.) or can be prepared via conventional antibody generation methods.

Western Blotting

To measure the effect of treatment with antisense oligonucleotides against TRX, protein levels of TRX in treated and untreated cells were determined using western blotting. After treatment with oligonucleotide as described in example 5, cells were harvested in ice-cold lysis buffer (50 mM Tris, pH 6,8, 10 mM NaF, 10% glycerol, 2,5% SDS, 0,1 mM natrium-orthovanadate, 10 mM β-glycerol phosphate, 10 mM dithiothreitol (DTT), Complete protein inhibitor cocktail (Boehringer Mannheim)). The lysate was stored at −80° C. until further use. Protein concentration of the protein lysate was determined using the BCA Protein Assay Kit (Pierce) as described by the manufacturer.

SDS Gel Electrophoresis

Protein samples prepared as described above were thawed on ice and denatured at 96° C. for 3 min. Samples were loaded on 1,0 mm 4-20% NuPage Tris-glycine gel (Invitrogen) and gels were run in TGS running buffer (BioRAD) in an Xcell II Mini-cell electrophoresis module (Invitrogen).

Semi-dry Blotting

After electrophoresis, the separated proteins were transferred to a polyvinyliden difluoride (PVDF) membrane by semi-dry blotting. The blotting procedure was carried out in a Semi-Dry transfer cell (CBS Scientific Co.) according to the manufacturers instructions. The membrane was stained with amidoblack to visualise transferred protein and was stored at 4° C. until further use.

Immunodetection

To detect the desired protein, the membrane was incubated with either polyclonal or monoclonal antibodies against the protein.

The membrane was blocked in blocking buffer (5% skim milk powder dissolved in PBST-buffer (150 mM NaCl, 10 mM Tris.base pH 7,4, 0,1% Tween-20)), washed briefly in PBS-buffer and incubated with primary antibody in blocking buffer at room temperature.

The following primary and secondary antibodies and concentrations/dilutions were used:

Monoclonal mouse anti-human thioredoxin antibody clone 2G11 (cat.# 559969, Pharmingen) 1:500

Monoclonal mouse anti-human tubulin Ab-4 (cat.# MS-719-P1, NeoMarkers)

Peroxidase-conjugated Goat Anti-Mouse Immunoglobulins (code no. P0447, DAKO) 1:1000

After incubation with the primary antibody the membrane was washed in PBS and incubated with a peroxidase conjugated secondary antibody at room temperature. The membrane was then washed in PBS followed by 3 additional 10 minutes washes in PBST with agitation at room temperature. After the last wash the membrane was incubated with ECL⁺ Plus reagent (Amersham) and chemiluminescens was detected using VersaDoc chemiluminescens detection system (BioRAD) or X-omat film (Kodak). The membrane was stripped in ddH₂O by incubation for 1 minutes at 96° C. After stripping, the membrane was put in PBS and stored at 4° C. (see FIG. 6 and 7. Compounds see Example 10)

### Example 10

In vitro Analysis: Antisense Inhibition of Human TRX Expression by Oligomeric Compound

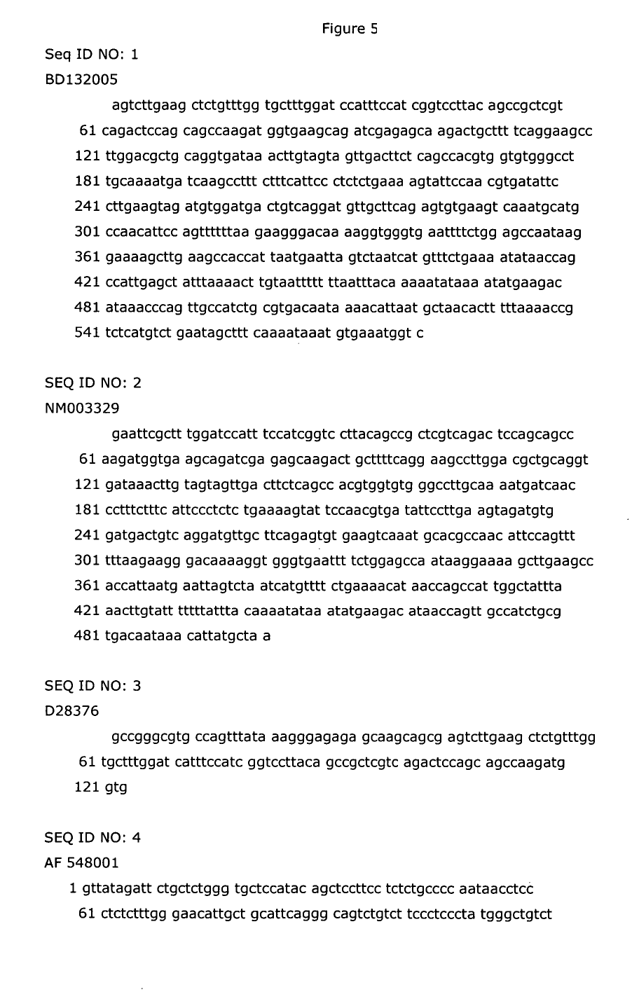

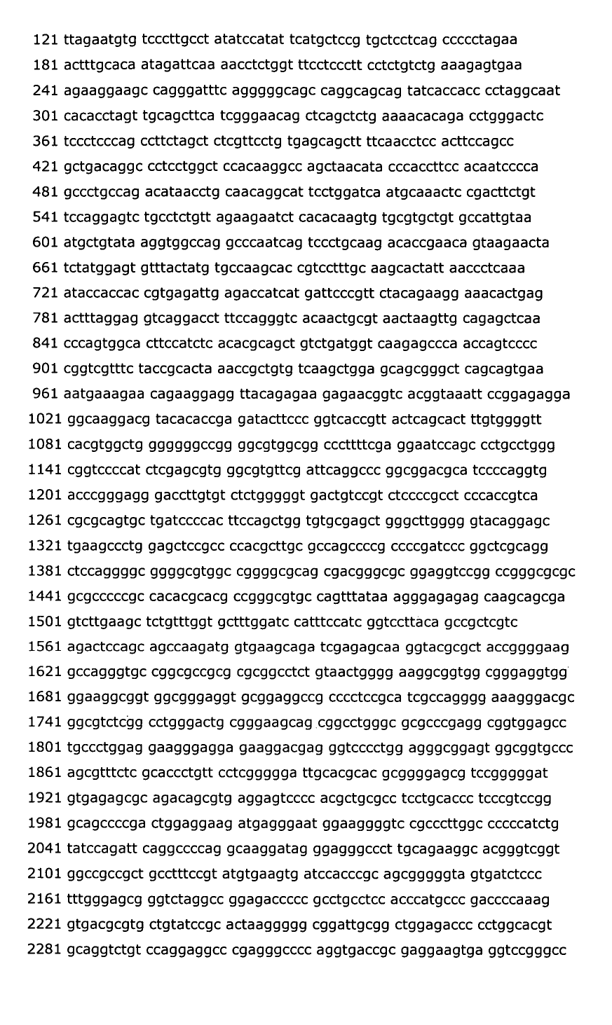

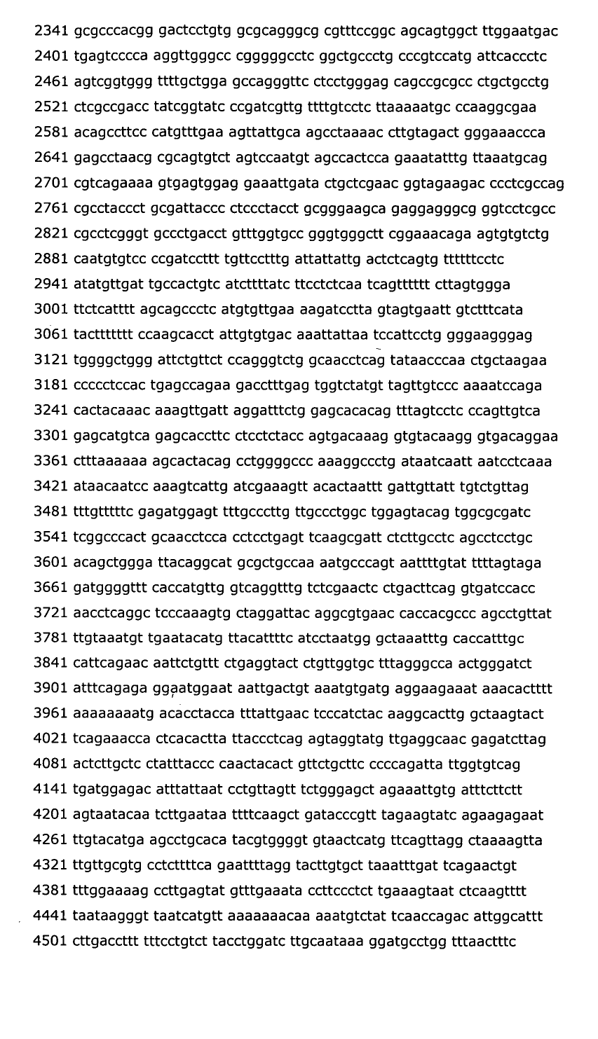

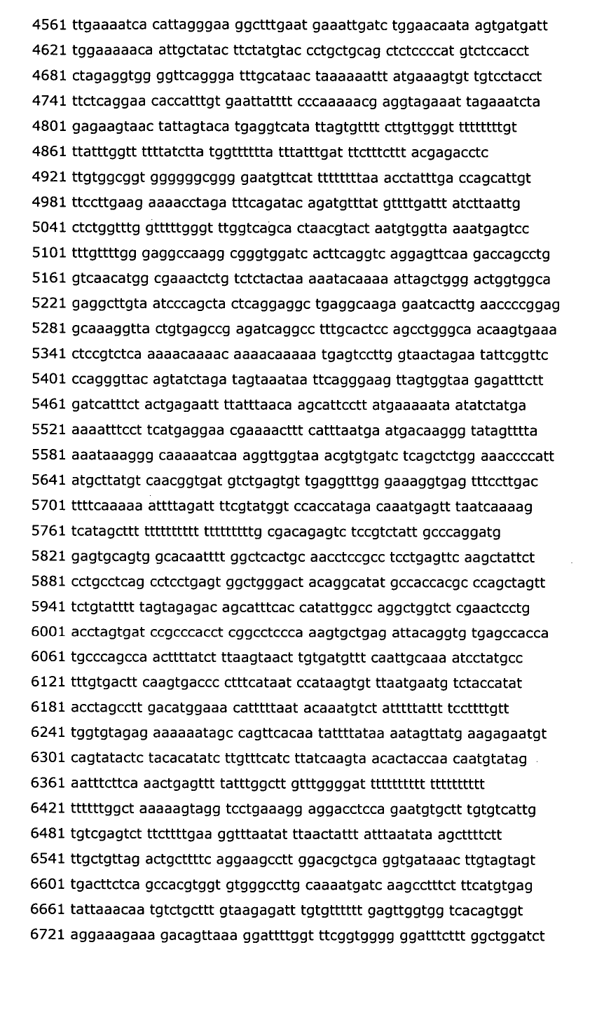

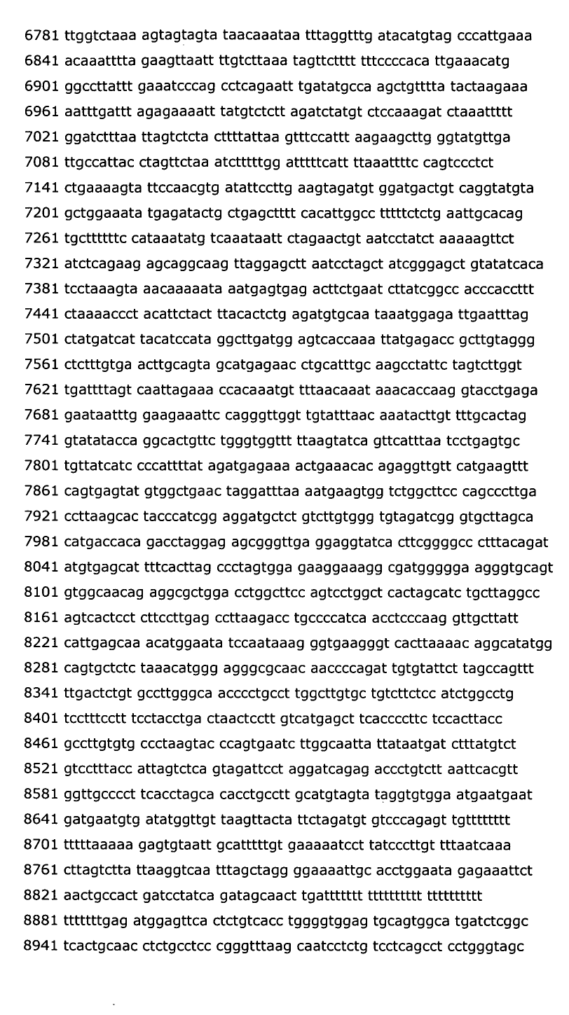

In accordance with the present invention, a series of oligonucleotides were designed to target different regions of the human TRX mRNA, using the published sequences (GenBank accession number, BD132005 incorporated herein as SEQ ID NO: 1, NM 003329 incorporated herein as SEQ ID NO: 2, D28376 incorporated herein as SEQ ID NO: 3, AF 548001 incorporated herein as SEQ ID NO: 4) (see FIG. 5). The oligonucleotides 16 nucleotides in length are shown in Table 2 having a CUR NO and a SEQ ID NO. “Target site” indicates the first nucleotide number on the particular target sequence to which the oligonucleotide binds. Table 3 shows IC50 of four compounds.

TABLE 2

Oligomeric compounds of the invention
Oligomeric compounds were evaluated for their potential to knockdown TRX mRNA in
15PC3 cells. The data are presented as percentage downregulation relative to mock
transfected cells. Transcript steady state was monitored by Real-time PCR and normalised
to the GAPDH transcript steady state. Note that all LNA C are 5′-Methyl-Cytosine.

| | | | | | |
|---|---|---|---|---|---|
| | | | | Specific design of Oligomeric | |
| | | | | compound | |
| | | Oligomeric | | Capital letters β-D-oxy-LNA | |
| | Positions | compound | Seq ID + Desing | S = phosphorthioate | |
| | (complementary) | Sequence | & | O = —O—P(O)₂—O— | % Inhibition |
| SEQ ID NO | to SEQ ID | 5′-3′ | Internal NO | Small letters DNA sugar | at 25 nM oligo |

| | | | | | |
|---|---|---|---|---|---|
| 5 | 14/1 | TCCAAAGCACCAAACA | 5A | TₛCₛCₛAₛaₛaₛgₛcₛaₛcₛcₛaₛAₛAₛCₛA | 72 |
| | | | CUR2672 | | |
| | | | 5B | TₛCₛCₛAₛaₛaₛgₛcₛaₛcₛcₛaₛAₛAₛCₛa | |
| | | | 5C | T_{O}C_{O}C_{O}A_{O}aₛaₛgₛcₛaₛcₛcₛaₛA_{O}A_{O}C_{O}A | |
| 6 | 33/1 | AAGGACCGATGGAAAT | 6A | AₛAₛGₛGₛaₛcₛcₛgₛcₛtₛgₛgₛAₛAₛAₛT | 68 |
| | | | CUR2673 | | |
| | | | 6B | AₛAₛGₛGₛaₛcₛcₛgₛaₛtₛgₛgₛAₛAₛAₛt | |
| | | | 6C | A_{O}A_{O}G_{O}G_{O}aₛcₛcₛgₛaₛtₛgₛgₛA_{O}A_{O}A_{O}T | |
| 7 | 206/1 | TTTTCAGAGAGGGAAT | 7A | TₛTₛTₛTₛcₛaₛgₛaₛgₛaₛgₛgₛGₛAₛAₛT | 49 |
| | | | CUR2674 | | |
| | | | 7B | TₛTₛTₛTₛcₛaₛgₛaₛgₛaₛgₛgₛGₛAₛAₛt | |
| | | | 7C | T_{O}T_{O}T_{O}T_{O}cₛaₛgₛaₛgₛaₛgₛgₛG_{O}A_{O}A_{O}T | |
| 8 | 229/1 | CAAGGAATATCACGTT | 8A | CₛAₛAₛGₛgₛaₛaₛtₛaₛtₛcₛaₛCₛGₛTₛT | >95 |
| | | | CUR2675 | | |
| | | | 8B | CₛAₛAₛGₛgₛaₛaₛtₛaₛtₛcₛaₛCₛGₛTₛt | 93 |
| | | | CUR2766 | | |
| | | | 8C | C_{O}A_{O}A_{O}G_{O}gₛaₛaₛtₛaₛtₛcₛaₛC_{O}G_{O}T_{O}T | |
| 9 | 281/2 | TGGAATGTTGGCGTGC | 9A | TₛGₛGₛAₛaₛtₛgₛtₛtₛgₛgₛcₛGₛTₛGₛC | >95 |
| | | | CUR2676 | | |
| | | | 9B | TₛGₛGₛAₛaₛtₛgₛtₛtₛgₛgₛcₛGₛTₛGₛc | |
| | | | 9C | T_{O}G_{O}G_{O}A_{O}aₛtₛgₛtₛtₛgₛgₛcₛG_{O}T_{O}G_{O}C | |
| 10 | 347/1 | TCCTTATTGGCTCCAG | 10A | TₛCₛCₛTₛtₛaₛtₛtₛgₛgₛcₛtₛCₛCₛAₛG | 84 |
| | | | CUR2677 | | |
| | | | 10B | TₛCₛCₛTₛtₛaₛtₛtₛgₛgₛcₛtₛCₛCₛAₛG | |
| | | | 10C | T_{O}C_{O}C_{O}T_{O}tₛaₛtₛtₛgₛgₛcₛtₛC_{O}C_{O}A_{O}G | |
| 11 | 73/1 | GCTTCACCATCTTGGC | 11A | GₛCₛTₛTₛcₛaₛcₛcₛaₛtₛcₛtₛTₛGₛGₛC | 31 |
| | | | CUR2678 | | |
| | | | 11B | GₛCₛTₛTₛcₛaₛcₛcₛaₛtₛcₛtₛTₛGₛGₛc | |
| | | | 11C | G_{O}C_{O}T_{O}T_{O}cₛaₛcₛcₛaₛtₛcₛtₛT_{O}G_{O}G_{O}C | |
| 12 | 46/1 | GACGAGCGGCTGTAAG | 12A | GₛAₛCₛGₛaₛgₛcₛgₛgₛcₛtₛgₛTₛAₛAₛG | 74 |
| | | | CUR2679 | | |
| | | | 12B | GₛAₛCₛGₛaₛgₛcₛgₛgₛcₛtₛgₛTₛAₛAₛg | |
| | | | 12C | G_{O}A_{O}C_{O}G_{O}aₛgₛcₛgₛgₛcₛtₛgₛT_{O}A_{O}A_{O}G | |
| 13 | 167/1 | CAAGGCCCACACCACG | 13A | CₛAₛAₛGₛgₛcₛcₛcₛaₛcₛaₛcₛCₛAₛCₛG | 71 |
| | | | CUR2680 | | |
| | | | 13B | CₛAₛAₛGₛgₛcₛcₛcₛaₛcₛaₛcₛCₛAₛCₛg | |
| | | | 13C | C_{O}A_{O}A_{O}G_{O}gₛcₛcₛcₛaₛcₛaₛcₛC_{O}A_{O}C_{O}G | |
| 14 | 136/1 | CTACTACAAGTTTATC | 14A | CₛTₛAₛCₛtₛaₛcₛaₛaₛgₛtₛtₛTₛAₛTₛC | 78 |
| | | | CUR2681 | | |
| | | | 14B | CₛTₛAₛCₛtₛaₛcₛaₛaₛgₛtₛtₛTₛAₛTₛc | |
| | | | 14C | C_{O}T_{O}A_{O}C_{O}tₛaₛcₛaₛaₛgₛtₛtₛT_{O}A_{O}T_{O}C | |
| 15 | 91/1 | CAGTCTTGCTCTCGAT | 15A | CₛAₛGₛTₛcₛtₛtₛgₛcₛtₛcₛtₛCₛGₛAₛT | 61 |
| | | | CUR2682 | | |
| | | | 15B | CₛAₛGₛTₛcₛtₛtₛgₛcₛtₛcₛtₛCₛGₛAₛT | |
| | | | 15C | C_{O}A_{O}G_{O}T_{O}cₛtₛtₛgₛcₛtₛcₛtₛC_{O}G_{O}A_{O}T | |
| 16 | 262/1 | AAGCAACATCCTGACA | 16A | AₛAₛGₛCₛaacatcctGₛAₛCₛA | |
| | | | 16B | AₛAₛGₛCₛaacatcctGₛAₛCₛa | |
| | | | 16C | A_{O}A_{O}G_{O}C_{O}aacatcctG_{O}A_{O}C_{O}A | |
| 17 | 1815/4 | CTCGTCCTTCTCCTCC | 17A | CₛTₛCₛGₛtₛcₛcₛtₛtₛcₛtₛcₛCₛTₛCₛC | 49 |
| | (intron) | | CUR2767 | | |
| | | | 17B | CₛTₛCₛGₛtₛcₛcₛtₛtₛcₛtₛcₛCₛTₛCₛc | |
| | | | 17C | C_{O}T_{O}C_{O}G_{O}tₛcₛcₛtₛtₛcₛtₛcₛC_{O}T_{O}C_{O}C | |
| 18 | 1988/4 | CATCTTCCTCCAGTCG | 18A | CₛAₛTₛCₛtₛtₛcₛcₛtₛcₛcₛaₛGₛTₛCₛG | 45 |
| | (intron) | | CUR2768 | | |
| | | | 18B | CₛAₛTₛCₛtₛtₛcₛcₛtₛcₛcₛaₛGₛTₛCₛg | |
| | | | 18C | C_{O}A_{O}T_{O}C_{O}tₛtₛcₛcₛtₛcₛcₛaₛG_{O}T_{O}C_{O}G | |
| 19 | 1/1 | ACAGAGCTTCAAGACT | | | |
| 20 | 17/1 | GGATCCAAAGCACCAA | | | |
| 21 | 33/1 | AAGGACCGATGGAAAT | | | |
| 22 | 49/1 | TCTGACGAGCGGCTGT | | | |
| 23 | 65/1 | ATCTTGGCTGCTGGAG | | | |
| 24 | 81/1 | CTCGATCTGCTTCACC | | | |
| 25 | 97/1 | GAAAAGCAGTCTTGCT | | | |
| 26 | 113/1 | GCGTCCAAGGCTTCCT | | | |
| 27 | 129/1 | AAGTTTATCACCTGCA | | | |
| 28 | 145/1 | AGAAGTCAACTACTAC | | | |
| 29 | 161/1 | CCACACCACGTGGCTG | | | |
| 30 | 177/1 | GATCATTTTGCAAGGC | | | |
| 31 | 193/1 | AATGAAAGAAAGGCTT | | | |
| 32 | 209/1 | TACTTTTCAGAGAGGG | | | |
| 33 | 225/1 | GAATATCACGTTGGAA | | | |
| 34 | 241/1 | CCACATCTACTTCAAG | | | |
| 35 | 257/1 | ACATCCTGACAGTCAT | | | |
| 36 | 273/1 | TTCACACTCTGAAGCA | | | |
| 37 | 289/1 | TTGGCATGCATTTGAC | | | |
| 38 | 305/1 | TTAAAAAACTGGAATG | | | |
| 39 | 321/1 | CACCTTTTGTCCCTTC | | | |
| 40 | 337/1 | CTCCAGAAAATTCACC | | | |
| 41 | 353/1 | AGCTTTTCCTTATTGG | | | |
| 42 | 369/1 | ATTAATGGTGGCTTCA | | | |
| 43 | 385/1 | ATGATTAGACTAATTC | | | |
| 44 | 401/1 | TTATATTTTCAGAAAC | | | |
| 45 | 417/1 | ATAGCTCAATGGCTGG | | | |
| 46 | 433/1 | AAATTACAAGTTTTAA | | | |
| 47 | 449/1 | TTTTTGTAAATTAAAA | | | |
| 48 | 465/1 | GTCTTCATATTTTATA | | | |
| 49 | 481/1 | TGGCAACTGGGTTTAT | | | |
| 50 | 497/1 | TTTATTGTCACGCAGA | | | |
| 51 | 513/1 | GTGTTAGCATTAATGT | | | |
| 52 | 529/1 | GAGACGGTTTTAAAAA | | | |
| 53 | 545/1 | AAAGCTATTCAGACAT | | | |
| 54 | 561/1 | TTTCACATTTATTTTG | | | |
| 55 | 25/3 | CGCTGCTTGCTCTCTC | | | |
| 56 | 9/3 | CCTTTATAAACTGGCA | | | |
| 57 | 1/3 | AACTGGCACGCCCGGC | | | |

TABLE 3
IC₅₀ (nM) in two cell lines of different origin.
Oligomeric compounds were evaluated for their potential to
knockdown TRX mRNA in 15PC3 cells. Transcript steady
state was monitored by Real-time PCR and normalised to
the GAPDH transcript steady state. Note that all
LNA C are 5′-Methyl-Cytosine.

| Cell line/Oligo | MCF7 | 15PC3 |
|---|---|---|
| CUR2675 (8A) | <2 | <1 |
| CUR2676 (9A) | <3 | <0.5 |
| CUR2677 (10A) | <5 | <0.5 |
| CUR2681 (14A) | <35 | <2 |
| CUR2766 (8B) | | <1 |

As showed in table 2 and 3, SEQ ID NO 5, 6, 7, 8, 9, 10, 11, 12, 13, 14, 15, 17 and 18 demonstrated at least 30% inhibition of survivin expression in at 25 nM and are therefore preferred. Compounds of particular interest are 8A, 9A, 10A and 14A, which have shown a low IC₅₀.

Specificity of LNA oligomeric compounds targeting TRX were also tested. 15PC3 cells were transfected with LNA oligos targeting either human survivin (4LNA/PS+8PS+4LNA/PS) (named LNA survivin) or human thioredoxin (CUR2766) at 5 nM and 25 nM (see FIG. 8)

### Example 11

Apoptosis Induction by LNA Antisense Oligomeric Compounds Targeting Trx

Measurement of apoptosis using BD™ cytometric bead array (CBA) (cat 557816). Cells were transfected using lipofectamine 2000 as described (see Example 5). 24 h following transfection, the cells from the supernatant was spun down and the adherent cells were trypsionised and spun down. The cell pellet was resuspended/washed in PBS and counted to bring cell concentration to 2×10⁶ cells/ml lysis buffer containing protease inhibitors. The procedure was proceeded as described by manufacturer with the following modifications. When cells were lysed, they were lysed for 40 min and vortexed with a 10 min interval. 1×10⁵ cells were incubated with Caspase 3 beads, mixed briefly and incubated for 1 h at room temperature, before they were analysed by flow cytometri. The data were analysed using the BDTM CBA software, transferred to Excel where all data were related to mock (which is set to one). (see FIG. 9).

### Example 12

Antisense Oligonucleotide Inhibition of TRX in Proliferating Cancer Cells

Cells were seeded to a density of 12000 cells per well in white 96 well plate (Nunc 136101) in DMEM the day prior to transfection. The next day cells were washed once in prewarmed OptiMEM followed by addition of 72 □l OptiMEM containing 5 □g/ml Lipofectamine2000 (In vitrogen). Cells were incubated for 7 min before adding 18 □l oligonucleotides diluted in OptiMEM. The final oligonucleotide concentration ranged from 5 nM to 100 nM. After 4 h of treatment, cells were washed in OptiMEM and 100 □l serum containing DMEM was added. Following oligo treatment cells were allowed to recover for the period indicated, viable cells were measured by adding 20 □l the tetrazolium compound [3-(4,5-dimethyl-2-yl)-5-(3-carboxymethoxyphenyl)-2-(4-sulfophenyl)-2H-tetrazolium, inner salt; MTS] and an electron coupling reagent (phenazine ethosulfate; PES) (CellTiter 96® AQᵤₑₒᵤₛ One Solution Cell Proliferation Assay, Promega). Viable cells were measured at 490 nm in a Powerwave (Biotek Instruments). Growth rate (□OD/h) were plotted against oligo concentration.

### Example 13

Measurement of Ploidy (Cell Cycle) and DNA Degradation (apoptosis) of Cells Following Treatment with Oligomeric Compounds Targeting Trx

The late stage in the apoptotic cascade leads to large numbers of small fragments of DNA which can be analysed by propidium iodide staining of the cells, furthermore, propidium iodide staining can be used to asses ploidy in treated cells. To assess ploidy/apoptosis of cells treated with oligomeric compound directed against TRX, cells were washed in PBA and fixed for 1 h in 70% EtOH at 4° C. After treatment with 50 μg/ml RNAse (Sigma) for 20 min at room temperature cells were washed with PBS and incubated with 40 μg/ml propidium iodide (Sigma or BD) for 30 min. All samples were analysed using fluorescence activated cell sorter (FACSCalibur, Becton Dickinson) and Cell Quest software. In the DNA histogram the hypodiploid or the sub-G1 peak represented the apoptotic cells.

### Example 14

Measurement of Changes in the Mitochondrial Membrane Potential of Cells Following Treatment with Oligomeric Compounds Targeting Trx

To measure changes in the mitochondrial membrane potential the MitoSensor™ reagent method (Becton Dickinson, Cat # K2017-1) was used. MitoSensor™ reagent is taken up by healthy cells, in which it forms aggregates that emit red fluorescence. Upon apoptosis the mitochondrial membrane potential changes and does not allow the reagent to aggregate within the mitochondria and therefore it remains in the cytoplasm in its monomeric form where it emits green fluorescence. Cells treated with oligomeric compounds directed against TRX were washed and incubated in MitoSensor Reagent diluted in Incubation buffer as described by manufacturer. Changes in membrane potential following oligo treatment was detected by fluorescence activated cell sorter (FACSCalibur, Becton Dickinson) and by the use of Cell Quest software.

### Example 15

Inhibition of Capillary Formation of Endothelial Cells Following Antisense Oligo Treatment

Endothelial monolayer cells (e.g. HUVEC) were incubated with antisense oligos directed against survivin. Tube formation was analysed by either of the two following methods. The first method was the BD BioCoat angiogenesis tube formation system. Cells were transfected with oligos as described (example 5). Transfected cells were seeded at 2×10⁴ cells/96 well onto matrigel polymerized BD Biocoat angiogeneis plates. The plates were incubated for the hours/days indicated with or without PMA (5-50 nM), VEGF (20-200 ng/ml), Suramin or vechicle. The plates were stained with Cacein AM as stated by the manufacturer and images were taken. Total tube length was measured using MetaMorph.

Althernatively, cells were seeded in rat tail type I collagen (3 mg/ml, Becton Dickinson) in 0.1 volumen of 10×DMEM, neutralised with sterile 1 M NaOH and kept on ice or in matrigel. Cells were added to the collagen suspension at a final concentration of 1×10⁶ cells/ml collagen. The cell-collagen mixture was added to 6-well or 35 mm plates and placed in a humidified incubator at 37° C. When geled 3 ml of culture medium plus an extra 10% FBS were added and cells were allow to form capillary-like vascular tubes over the period indicated in the presence or absence of PMA (16 nM), VEGF (50 ng/ml). Tube formation was quantified following cryostat sectioning of the gels and examination of sections by phase-contrast microscopy.

### Example 16

Measurement of in vitro Cytotoxicity Following Treatment with Oligomeric Compounds Targeting Trx

Cells were seeded (0.3-1.2×10⁴) and treated with antisense oligos as described (example for MTS assay Exampel 12). At the times indicated, 20-50 □l medium from the antisense treated cells were transferred to 96-well plates in order to measure the release of LDH to the medium. An equal volume of LHD substrate was added as described by the manufacturer. Released LDH was measured using a 30-minute coupled enzymatic assay, which results in the conversion of a tetrazolium salt (INT) into a red formazan product. The amount of colour formed is proportional to the number of lysed cells. Visible wavelength absorbance data (measured at 490 nm) were collected using a standard 96-well plate reader (Powerwave, Bio-Tek Instruments). As positive control cells were treated for about 45 minutes with 0,9% Triton X-100 (=100% lysis). Cytotoxicity was plotted relative to mock and Triton-x 100 treated cells (100% lysis=100% cytotoxicity).

### Example 17

In vivo Model: Tumour Growth Inhibition of Human Tumour Cells Grown in vivo by Systemic Treatment with Antisense Oligonucleotides

Female NMRI athymic nude mice of 6 weeks old were purchased from M&B, Denmark and allowed to acclimatize for at least one week before entering experiments. Human cancer cells typically 10⁶ cells suspended in 300 μl matrigel (BD Bioscience), were subcutaneously injected into the flanks of 7-8 week old NMRI athymic female nude mice. When the tumour growth was established, typically 7-12 days post tumour cell injection; different antisense oligonucleotides were administrated at 5 mg/kg/day for up to 28 days using ALZET osmotic pumps implanted subcutaneously. Prior to dorsal implantation the pumps were incubated overnight at room temperature in sterile PBS to start the pumps. Control animals received saline alone for the same period. Each experimental group included at least 5 mice. Anti-tumour activities were estimated by the inhibition of tumour volume. Tumour growth was followed regularly by measuring 2 perpendicular diameters. Tumour volumes were calculated according to the formula (π×L×D²/6), where L represents the largest diameter and D the tumour diameter perpendicular to L. At the end of treatment the animals were sacrificed and tumour weights were measured. Mean tumour volume and weights of groups were compared using Mann-Whitney's test. All analysis was made in SPSS version 11.0 for windows. Optimally a Western blot analysis may also be performed to measure if the antisense oligonucleotides have an inhibitory effect on protein levels. At the end of treatment period mice were therefore anaesthetised and the tumours were excised and immediately frozen in liquid nitrogen. The tumours were homogenized in lysis buffer (i.e. 20 mM Tris-Cl [pH 7.5]; 2% Triton X-100; 1/100 vol. Protease Inhibitor Cocktail Set III (Calbiochem); 1/100 vol. Protease Inhibitor Cocktail Set II (Calbiochem)) at 4° C. with the use of a motor-driven homogeniser. 500 μl lysis buffer was applied per 100 mg tumour tissue. Tumour lysates from each group of mice were pooled and centrifuged at 13.000 g for 5 min at 4° C. to remove tissue debris. Protein concentrations of the tumour extracts were determined using the BCA Protein Assay Reagent Kit (Pierce, Rockford).

The protein extracts (50-100 μg) were fractionated on a gradient SDS-PAGE gel spanning from 4-20% and transferred to PVDF membranes and visualized by aminoblack staining. The expression of TRX was detected with anti-human TRX antibody followed by horseradish peroxidase-conjugated anti-goat IgG (DAKO). Immunoreactivity was detected by the ECL Plus (Amersham biotech) and quantitated by a Versadoc 5000 lite system (Bio-Rad).

### Example 17a

In vivo Model: Tumor Growth Inhibition in a HT29 Human Colon Cancer Xenograft Model in Nude Mice Treated with LNA Oligomeric Compounds

Female NMRI athymic nude mice of 6 weeks old were purchased from M&B, Denmark and allowed to acclimatize for at least one week before entering experiments. Human cancer cells 3×10⁶ cells suspended in 300 μl matrigel (BD Bioscience), were subcutaneously injected into the flanks of 7-8 week old NMRI athymic female nude mice (at day 0). Each experimental group included at least 5 mice. The present study was performed to test the single effect of Cur2681 targeting thioredoxin in a HT29 human colon cancer xenograft model in nude mice. The antisense oligonucleotide administered 10 and 20 mg/kg s.c. day 7-20 by osmotic mini pumps. Efficacy was evaluated by measurement of tumour volume during the treatment period day 21. HT29, human colon cancer xenograft, BALB/c female nude mice. Mean/SEM. Mean tumour volumes and mean tumour weight observed in the different treatment groups were statistically compared by using the Mann Whitney test. (see FIG. 10)

### Example 18

In vivo Model: Tumor Growth Inhibition of Human Tumour Fragments Transplanted in Nude Mice after Intraperetoneal Treatment with LNA Oligomeric Compounds

Tumour growth inhibiting activity of LNA antisense oligonucleotides was tested in xenotransplanted athymic nude mice, NMRI nu/nu, from Oncotest's (Freiburg, Germany) breeding colony. Human tumour fragments from breast (MDA MB 231), prostate (PC3) or lung tumours (LXFE 397, Oncotest) were obtained from xenografts in serial passage in nude mice. After removal of tumors from donor mice, they were cut into fragments (1-2 mm diameter) and placed in RPMI 1640 culture medium until subcutaneous implantation. Recipient mice were anaesthetized by inhalation of isoflurane. A small incision was made in the skin of the back. The tumor fragments (2 fragments per mouse) were transplanted with tweezers. MDA MB 231 and LXFE 397 tumors were tarnsplanted in female mice, PC3 tumors were transplanted in male mice. When a mean tumour diameter 4-6 mm was reached, animals were randomized and treated with oligonucleotides at 20 mg/kg intraperetoneally once a day for three weeks excluding weekends. A vehicle (saline) and positive control group (Taxol, 20 mg/kg/day) were included in all experiments. All groups consisted of 6 mice. The tumour volume was determined by two-dimensional measurement with a caliper on the day of randomization (Day 0) and then twice weekly. Tumor volumes were calculated according to the formula: (a×b²)×0.5 where a represents the largest and b the perpendicular tumor diameter. Mice were observed daily for 28 days after randomization until tumour volume was doubled. Mice were sacrificed when the tumour diameters exceeded 1.6 cm. For the evaluation of the statistical significance of tumour inhibition, the U-test by Mann-Whitney-Wilcoxon was performed. By convention, p-values <0.05 indicate significance of tumor inhibition.

### Example 19

Biodistribution of Oligonucleotides in Mice

Female NMRI athymic nude mice of 6 weeks old were purchased from M&B, Denmark and allowed to acclimatize for at least one week before entering experiments. Human cancer cells typically 10⁶ cells suspended in 300 μl matrigel (BD Bioscience) were subcutaneously injected into the flanks of 7-8 week old NMRI athymic female nude mice. When tumour growth was evident, tritium labelled oligonucleotides were administrated at 5 mg/kg/day for 14 days using ALZET osmotic pumps implanted subcutaneously. The oligonucleotides were tritium labeled as described by Graham M J et al. (J Pharmacol Exp Ther 1998; 286(1): 447-458). Oligonucleotides were quantitated by scintillation counting of tissue extracts from all major organs (liver, kidney, spleen, heart, stomach, lungs, small intestine, large intestine, lymph nodes, skin, muscle, fat, bone, bone marrow) and subcutaneous transplanted human tumour tissue.

The present invention has been described with specificity in accordance with certain of its preferred embodiments. Therefore, the following examples serve only to illustrate the invention and are not intended to limit the same.

1. A compound consisting of a total of 8-50 nucleotides and/or nucleotidee analogues, wherein said compound comprises a subsequence of at least 8 nucleotides or nucleotide analogues, said subsequence being located within a sequence selected from the group consisting of SEQ ID NO: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56 or 57.

2. A compound of claim 1, which modulates the expression of thioredoxin.

3. A compound of claim 1 consisting of 8-50 nucleotides and/or nucleotidee analogues targeted to a nucleic acid molecule encoding TRX, wherein said compound specifically hybridises with a nucleic acid encoding TRX and inhibits the expression of TRX and wherein said compound comprises a subsequence of at least 8 nucleotides or nucleotide analogues, said subsequence being located within a sequence selected from SEQ ID NO: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57.

4. The compound according to claim 1, which is an antisense oligonucleotide.

5. The compound according to claim 1, comprising at least one nucleotide analogue.

6. The compound according to claim 1, comprising at least one Locked Nucleic Acid (LNA) unit.

7. The compound according to claim 6, wherein the Locked Nucleic Acid (LNA) unit has the structure of the general formula 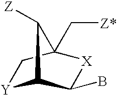
X and Y are independently selected among the groups —O—, —S—, —N(H)—, N(R)—, —CH₂— or —CH— (if part of a double bond), —CH₂—O—, —CH₂—S—, —CH₂—N(H)—, —CH₂—N(R)—, —CH₂—CH₂— or —CH₂—CH— (if part of a double bond), —CH═CH—, where R is selected form hydrogen and C₁₋₄-alkyl;
Z and Z* are independently absent, selected among an intemucleoside linkage, a terminal group or a protecting group;
B constitutes a natural or non-natural nucleobase;
and the asymmetric groups may be found in either orientation.

8. The compound according to claim 6 or 7, wherein at least one nucleotide comprises a Locked Nucleic Acid (LNA) unit according any of the formulas 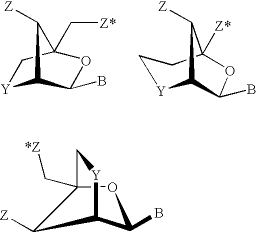
wherein Y is independently selected from —O—, —S—, —NH—, and N(R^{H});
Z and Z* are independently absent, selected among an intemucleoside linkage, a terminal group or a protecting group; and
B constitutes a natural or non-natural nucleobase.

9. The compound according to claim 1, wherein the nucleotide analogue comprises an intemucleoside linkage selected from the group consisting of —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —S—P(O)₂—S—, —O—PO(R H)—O—, O—PO(OCH₃)—O—, —O—PO(NR^{H})—O—, —O—PO(OCH₂CH₂S-R)—O—, —O—PO(BH₃)—O—, —O—PO(NHR^{H})—O—, —O—P(O)₂—NR^{H}—, —NR^{H}—P(O)₂—O—, —NR^{H}—CO—O, where R^{H} is selected form hydrogen and C₁₋₄-alkyl.

10. The compound according to claim 1, wherein the nucleotide analogue comprises a modified nucleobases selected from the group consisting of 5-methylcytosine, isocytosine, pseudoisocytosine, 5-bromouracil, 5-propynyluracil, 6-aminopurine, 2-aminopurine, inosine, diaminopurine, 2-chloro-6-aminopurine.

11. The compound according to claim 5, wherein the LNA is oxy-LNA, thio-LNA, amino-LNA in either the D-β or L-α configurations or combinations thereof.

12. A compound consisting of a total of 8-50 nucleotides and/or nucleotidee analogues, targeted to a nucleic acid molecule encoding TRX, wherein said compound specifically hybridises with a nucleic acid encoding TRX and inhibits the expression of TRX and wherein said compound comprises a subsequence of at least 8 nucleotides or nucleotide analogues SEQ ID NO: 5, 6, 7, 8, 9, 10, 11, 12, 13, 14, 15, 16, 17, or 18.

13. The compound according to claim 4, wherein the antisense oligonucleotide is a design according to any of the designs presented in FIG. 1.

14. The compound according to claim 13, wherein the antisense oligonucleotide is a gapmer.

15. The compound according to claim 1, wherein the antisense oligonucleotide is a 13, 14, 15, 16, 17, 18, 19, 20 or 21-mer in length.

16. The compound according to claim 1, comprising at least 2 LNA units, such as 2, 3, 4, 5, 6, 7, 8, 9, 10, 11, 12, 13, 14, 15, 16, 17, 18, 19, 20 or 21 LNA units.

17. The compound according to claim 1, wherein the subsequence is SEQ ID NO: 2.

18. The compound according to claim 1, wherein the subsequence is SEQ ID NO: 3.

19. The compound according to claim 1, wherein the subsequence is SEQ ID NO: 4.

20. The compound according to claim 1, wherein the subsequence is SEQ ID NO: 68.

21. The compound according to claim 1, wherein the subsequence is SEQ ID NO: 71.

22. The compound according to claim 1, wherein the subsequence is SEQ ID NO: 74.

23. The compound according to claim 1, wherein the subsequence is SEQ ID NO: 77.

24. The compound according to claim 1, wherein the subsequence is SEQ ID NO: 80.

25. The compound according to claim 1, wherein the subsequence is SEQ ID NO: 83.

26. The compound according to claim 1, wherein the subsequence is SEQ ID NO: 86.

27. The compound according to claim 1, wherein the subsequence is SEQ ID NO: 89.

28. The compound according to claim 1, wherein the subsequence is SEQ ID NO: 92.

29. The compound according to claim 1, wherein the subsequence is SEQ ID NO: 95.

30. The compound according to claim 1, wherein the subsequence is SEQ ID NO: 98.

31. The compound according to claim 1, wherein the 3′ end LNA is replaced by the corresponding natural nucleoside.

32. A compound consisting of SEQ ID NO: 2.

33. A compound consisting of SEQ ID NO: 3.

34. A compound consisting of SEQ ID NO: 4.

35. A compound consisting of SEQ ID NO: 68.

36. A compound consisting of SEQ ID NO: 71.

37. A compound consisting of SEQ ID NO: 74.

38. A compound consisting of SEQ ID NO: 77.

39. A compound consisting of SEQ ID NO: 80.

40. A compound consisting of SEQ ID NO: 83.

41. A compound consisting of SEQ ID NO: 86.

42. A compound consisting of SEQ ID NO: 89.

43. A compound consisting of SEQ ID NO: 92.

44. A compound consisting of SEQ ID NO: 95.

45. A compound consisting of SEQ ID NO: 98.

50. The compound according to any of claims 34-45, wherein the 3′ end LNA is replaced by the corresponding nucleotide.

51. A conjugate comprising the compound according to claim 1 and at least one non-nucleotide or non-polynucleotide moiety covalently attached to said compound.

52. A pharmaceutical composition comprising a compound as defined in claim 1 or a conjugate as defined in claim 59, and a pharmaceutically acceptable diluent, carrier or adjuvant.

53. The pharmaceutical composition according to claim 51 further comprising at least one chemotherapeutic agent.

54. The pharmaceutical composition according to claim 52, wherein said chemotherapeutic compound is selected from the group consisting of adrenocorticosteroids, such as prednisone, dexamethasone or decadron; altretamine (hexalen, hexamethylmelamine (HMM)); amifostine (ethyol); aminoglutethimide (cytadren); amsacrine (M-AMSA); anastrozole (arimidex); androgens, such as testosterone; asparaginase (elspar); bacillus calmette-gurin; bicalutamide (casodex); bleomycin (blenoxane); busulfan (myleran); carboplatin (paraplatin); carmustine (BCNU, BiCNU); chlorambucil (leukeran); chlorodeoxyadenosine (2-CDA, cladribine, leustatin); cisplatin (platinol); cytosine arabinoside (cytarabine); dacarbazine (DTIC); dactinomycin (actinomycin-D, cosmegen); daunorubicin (cerubidine); docetaxel (taxotere); doxorubicin (adriomycin); epirubicin; estramustine (emcyt); estrogens, such as diethylstilbestrol (DES); etopside (VP-16, VePesid, etopophos); fludarabine (fludara); flutamide (eulexin); 5-FUDR (floxuridine); 5-fluorouracil (5-FU); gemcitabine (gemzar); goserelin (zodalex); herceptin (trastuzumab); hydroxyurea (hydrea); idarubicin (idamycin); ifosfamide; IL-2 (proleukin, aldesleukin); interferon alpha (intron A, roferon A); irinotecan (camptosar); leuprolide (lupron); levamisole (ergamisole); lomustine (CCNU); mechlorathamine (mustargen, nitrogen mustard); melphalan (alkeran); mercaptopurine (purinethol, 6-MP); methotrexate (mexate); mitomycin-C (mutamucin); mitoxantrone (novantrone); octreotide (sandostatin); pentostatin (2-deoxycoformycin, nipent); plicamycin (mithramycin, mithracin); prorocarbazine (matulane); streptozocin; tamoxifin (nolvadex); taxol (paclitaxel); teniposide (vumon, VM-26); thiotepa; topotecan (hycamtin); tretinoin (vesanoid, all-trans retinoic acid); vinblastine (valban); vincristine (oncovin) and vinorelbine (navelbine).

55. A pharmaceutical composition comprising the compound of claim 1, which further comprises a pharmaceutically acceptable carrier.

56. A pharmaceutical composition comprising the compound of claim 1, which is employed in a pharmaceutically acceptable salt.

57. A pharmaceutical composition comprising the compound of claim 1, which is constitutes a pro-drug.

58. A pharmaceutical composition comprising the compound of claim 1, which further comprises an antiinflamatory compounds and/or antiviral compounds.

59. Use of a compound as defined in claim 1 or as conjugate as defined in claim 51 for the manufacture of a medicament for the treatment of cancer.

60. Use according to claim 59, wherein said cancer is in the form of a solid tumor.

61. Use according to claim 59 or 60, wherein said cancer is a carcinoma.

62. Use according to claim 61, wherein said carcinoma is selected from the group consisting of malignant melanoma, basal cell carcinoma, ovarian carcinoma, breast carcinoma, non-small cell lung cancer, renal cell carcinoma, bladder carcinoma, recurrent superficial bladder cancer, stomach carcinoma, prostatic carcinoma, pancreatic carcinoma, lung carcinoma, cervical carcinoma, cervical dysplasia, laryngeal papillomatosis, colon carcinoma, colorectal carcinoma and carcinoid tumors.

63. Use according to claim 62 wherein said carcinoma is selected from the group consisting of malignant melanoma, non-small cell lung cancer, breast carcinoma, colon carcinoma and renal cell carcinoma.

64. Use according to claim 63, wherein said malignant melanoma is selected from the group consisting of superficial spreading melanoma, nodular melanoma, lentigo maligna melanoma, acral melagnoma, amelanotic melanoma and desmoplastic melanoma.

65. Use according to claim 60 or 61, wherein said cancer is a sarcoma.

66. Use according to claim 65, wherein said sarcoma is selected from the group consisting of osteosarcoma, Ewing's sarcoma, chondrosarcoma, malignant fibrous histiocytoma, fibrosarcoma and Kaposi's sarcoma.

67. Use according to claim 60 or 61, wherein said cancer is a glioma.

68. A method for treating cancer, said method comprising administering a compound as defined in claim 1, a conjugate as defined in claim 51 or a pharmaceutical composition as defined in any of claims 52-58 to a patient in need thereof.

69. The method according to claim 68, wherein said cancer is in the form of a solid tumor.

70. The method according to claim 68 or 69, wherein said cancer is a carcinoma.

71. The method according to claim 70, wherein said carcinoma is selected from the group consisting of malignant melanoma, basal cell carcinoma, ovarian carcinoma, breast carcinoma, non-small cell lung cancer, renal cell carcinoma, bladder carcinoma, recurrent superficial bladder cancer, stomach carcinoma, prostatic carcinoma, pancreatic carcinoma, lung carcinoma, cervical carcinoma, cervical dysplasia, laryngeal papillomatosis, colon carcinoma, colorectal carcinoma and carcinoid tumors.

72. The method according to claim 71, wherein said carcinoma is selected from the group consisting of malignant melanoma, non-small cell lung cancer, breast carcinoma, colon carcinoma and renal cell carcinoma.

73. The method according to claim 72, wherein said malignant melanoma is selected from the group consisting of superficial spreading melanoma, nodular melanoma, lentigo maligna melanoma, acral melagnoma, amelanotic melanoma and desmoplastic melanoma.

74. The method according to claim 68, wherein said cancer is a sarcoma.

75. The method according to claim 74, wherein said sarcoma is selected from the group consisting of osteosarcoma, Ewing's sarcoma, chondrosarcoma, malignant fibrous histiocytoma, fibrosarcoma, artherosclerosis, psoriasis, diabetic retinopathy, rheumatoid arthritis, asthma, warts, allergic dermatitis and Kaposi's sarcoma.

75. The method according to claim 68, wherein said cancer is a glioma.

76. A method of inhibiting the expression of TRX, in cells or tissues comprising contacting said cells or tissues with the compound according to claim 1 so that expression of TRX is inhibited.

77. A method of modulating expression of a gene involved in a cancer disease comprising contacting the gene or RNA from the gene with an oligomeric compound wherein said compound has a sequence comprising at least an 8 nucleobase portion of SEQ ID NO: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56 or 57 whereby gene expression is modulated.

78. A method according to claim 77, wherein the compounds comprises one or more LNA units.

79. The method of claim 77 or 78, wherein the compound hybridizes with messenger RNA of the gene to inhibit expression thereof.

80. A method of treating a mammal suffering from or susceptible from an cancer disease, comprising:
administering to the mammal an therapeutically effective amount of an oligonucleotide targeted to TRX that comprises one or more LNA units.

81. The method according to any of the claims 77-80, wherein the cancer diseases is a lung, breast, colon, prostate, pancreas, lung, liver, thyroid, kidney, brain, testes, stomach, intestine, bowel, spinal cord, sinuses, bladder, urinary tract or ovaries cancer.

82. A method of modulating the red blood cell proliferation, cellular proliferation, ion metabolism, glucose and energy metabolism, pH regulation or matrix metabolism comprising contacting a cell with the antisense compound of claim 1 so that the cell is modulated.

83. A method of inhibiting the proliferation of cells comprising contacting cells in vitro with an effective amount of the antisense compound of claim 1, so that proliferation of the cells is inhibited.

84. The method of claim 83 wherein said cells are cancer cells.

85. A method of inhibiting the expression of TRX in human cells or tissues comprising contacting human cells or tissues with the compound of claim 1 so that expression of TRX is inhibited.

86. A method of treating an animal having a disease or condition associated with TRX comprising administering to an animal having a disease or condition associated with TRX a therapeutically or prophylactically effective amount of the antisense compound of claim 1 so that expression of TRX is inhibited.

87. The method of claim 86 wherein the disease or condition is a hyperproliferative condition.

88. The method of claim 87 wherein the hyperproliferative condition is cancer.

89. A method of treating a human having a disease or condition characterized by a reduction in apoptosis comprising administering to a human having a disease or condition characterized by a reduction in apoptosis a prophylactically or therapeutically effective amount of the antisense compound of claim 1.

90. A method of modulating apoptosis in a cell comprising contacting a cell with the antisense compound of claim 1 so that apoptosis is modulated.

91. A method of modulating cytokinesis in a cell comprising contacting a cell with the antisense compound of claim 1 so that cytokinesis is modulated.

92. A method of modulating the cell cycle in a cell comprising contacting a cell with the antisense compound of claim 1 so that the cell cycle is modulated.

93. A method of inhibiting the proliferation of cells comprising contacting cells with an effective amount of the antisense compound of claim 1, so that proliferation of the cells is inhibited.


SEQUENCE LISTING (150 sequences)

SEQ ID NO: 1 - DNA, 581 bp, Homo sapiens
agtcttgaagctctgtttggtgctttggatccatttccatcggtccttacagccgctcgtcagactccagcagccaagatggtgaagcagatcgagagcaagactgcttttcaggaagccttggacgctgcaggtgataaacttgtagtagttgacttctcagccacgtggtgtgggccttgcaaaatgatcaagcctttctttcattccctctctgaaaagtattccaacgtgatattccttgaagtagatgtggatgactgtcaggatgttgcttcagagtgtgaagtcaaatgcatgccaacattccagttttttaagaagggacaaaaggtgggtgaattttctggagccaataaggaaaagcttgaagccaccattaatgaattagtctaatcatgtttctgaaaatataaccagccattgagctatttaaaacttgtaatttttttaatttacaaaaatataaaatatgaagacataaacccagttgccatctgcgtgacaataaaacattaatgctaacactttttaaaaccgtctcatgtctgaatagctttcaaaataaatgtgaaatggtc

SEQ ID NO: 2 - DNA, 501 bp, Homo sapiens
gaattcgctttggatccatttccatcggtccttacagccgctcgtcagactccagcagccaagatggtgaagcagatcgagagcaagactgcttttcaggaagccttggacgctgcaggtgataaacttgtagtagttgacttctcagccacgtggtgtgggccttgcaaaatgatcaaccctttctttcattccctctctgaaaagtattccaacgtgatattccttgaagtagatgtggatgactgtcaggatgttgcttcagagtgtgaagtcaaatgcacgccaacattccagttttttaagaagggacaaaaggtgggtgaattttctggagccaataaggaaaagcttgaagccaccattaatgaattagtctaatcatgttttctgaaaacataaccagccattggctatttaaacttgtatttttttatttacaaaatataaatatgaagacataaccagttgccatctgcgtgacaataaacattatgctaa

SEQ ID NO: 3 - DNA, 123 bp, Homo sapiens
gccgggcgtgccagtttataaagggagagagcaagcagcgagtcttgaagctctgtttggtgctttggatcatttccatcggtccttacagccgctcgtcagactccagcagccaagatggtg

SEQ ID NO: 4 - DNA, 15100 bp, Homo sapiens
gttatagattctgctctgggtgctccatacagctccttcctctctgccccaataacctccctctctttgggaacattgctgcattcagggcagtctgtcttccctccctatgggctgtctttagaatgtgtcccttgcctatatccatattcatgctccgtgctcctcagccccctagaaactttgcacaatagattcaaaacctctggtttcctcccttcctctgtctgaaagagtgaaagaaggaagccagggatttcagggggcagccaggcagcagtatcaccacccctaggcaatcacacctagttgcagcttcatcgggaacagctcagctctgaaaacacagacctgggactctccctcccagccttctagctctcgttcctgtgagcagcttttcaacctccacttccagccgctgacaggccctcctggctccacaaggccagctaacatacccaccttccacaatccccagccctgccagacataacctgcaacaggcattcctggatcaatgcaaactccgacttctgttccaggagtctgcctctgttagaagaatctcacacaagtgtgcgtgctgtgccattgtaaatgctgtataaggtggccaggcccaatcagtccctgcaagacaccgaacagtaagaactatctatggagtgtttactatgtgccaagcaccgtcctttgcaagcactattaaccctcaaaataccaccaccgtgagattgagaccatcatgattcccgttctacagaaggaaacactgagactttaggaggtcaggacctttccagggtcacaactgcgtaactaagttgcagagctcaacccagtggcacttccatctcacacgcagctgtctgatggtcaagagcccaaccagtcccccggtcgtttctaccgcactaaaccgctgtgtcaagctggagcagcgggctcagcagtgaaaatgaaagaacagaaggaggttacagagaagagaacggtcacggtaaattccggagaggaggcaaggacgtacacaccgagatacttcccggtcaccgttactcagcactttgtggggttcacgtggctgggggggccggggcgtggcggcccttttcgaggaatccagccctgcctgggcggtccccatctcgagcgtgggcgtgttcgattcaggcccggcggacgcatccccaggtgacccgggagggaccttgtgtctctgggggtgactgtccgtctccccgcctcccaccgtcacgcgcagtgctgatccccacttccagctggtgtgcgagctgggcttgggggtacaggagctgaagccctggagctccgccccacgcttgcgccagccccgccccgatcccggctcgcaggctccaggggcggggcgtggccggggcgcagcgacgggcgcggaggtccggccgggcgcgcgcgcccccgccacacgcacgccgggcgtgccagtttataaagggagagagcaagcagcgagtcttgaagctctgtttggtgctttggatccatttccatcggtccttacagccgctcgtcagactccagcagccaagatggtgaagcagatcgagagcaaggtacgcgctaccggggaaggccagggtgccggcgccgcgcgcggcctctgtaactggggaaggcggtggcgggaggtggggaaggcggtggcgggaggtgcggaggccgcccctccgcatcgccaggggaaagggacgcggcgtctcggcctgggactgcgggaagcagcggcctgggcgcgcccgaggcggtggagcctgccctggaggaagggaggagaaggacgagggtcccctggagggcggagtggcggtgcccagcgtttctcgcaccctgttcctcgggggattgcacgcacgcggggagcgtccgggggatgtgagagcgcagacagcgtgaggagtccccacgctgcgcctcctgcaccctcccgtccgggcagccccgactggaggaagatgagggaatggaaggggtccgcccttggccccccatctgtatccagattcaggccccaggcaaggatagggagggcccttgcagaaggcacgggtcggtggccgccgctgcctttccgtatgtgaagtgatccacccgcagcgggggtagtgatctccctttgggagcgggtctaggccggagacccccgcctgcctccacccatgcccgaccccaaaggtgacgcgtgctgtatccgcactaagggggcggattgcggctggagaccccctggcacgtgcaggtctgtccaggaggcccgagggccccaggtgaccgcgaggaagtgaggtccgggccgcgcccacgggactcctgtggcgcagggcgcgtttccggcagcagtggctttggaatgactgagtccccaaggttgggcccgggggcctcggctgccctgcccgtccatgattcaccctcagtcggtgggttttgctggagccagggttcctcctgggagcagccgcgccctgctgcctgctcgccgacctatcggtatcccgatcgttgttttgtcctcttaaaaatgcccaaggcgaaacagccttcccatgtttgaaagttattgcaagcctaaaaccttgtagactgggaaacccagagcctaacgcgcagtgtctagtccaatgtagccactccagaaatatttgttaaatgcagcgtcagaaaagtgagtggaggaaattgatactgctcgaacggtagaagacccctcgccagcgcctaccctgcgattacccctccctacctgcgggaagcagaggagggcgggtcctcgcccgcctcgggtgccctgacctgtttggtgccgggtgggcttcggaaacagaagtgtgtctgcaatgtgtccccgatccttttgttcctttgattattattgactctcagtgttttttcctcatatgttgattgccactgtcatcttttatcttcctctcaatcagttttttcttagtgggattctcattttagcagccctcatgtgttgaaaagatccttagtagtgaattgtctttcatatactttttttccaagcacctattgtgtgacaaattattaatccattcctggggaagggagtggggctgggattctgttctccagggtctggcaacctcagtataacccaactgctaagaaccccctccactgagccagaagacctttgagtggtctatgttagttgtcccaaaatccagacactacaaacaaagttgattaggatttctggagcacacagtttagtcctcccagttgtcagagcatgtcagagcaccttcctcctctaccagtgacaaaggtgtacaagggtgacaggaactttaaaaaaagcactacagcctggggcccaaaggccctgataatcaattaatcctcaaaataacaatccaaagtcattgatcgaaagttacactaatttgattgttatttgtctgttagtttgtttttcgagatggagttttgcccttgttgccctggctggagtacagtggcgcgatctcggcccactgcaacctccacctcctgagttcaagcgattctcttgcctcagcctcctgcacagctgggattacaggcatgcgctgccaaaatgcccagtaattttgtatttttagtagagatggggtttcaccatgttggtcaggtttgtctcgaactcctgacttcaggtgatccaccaacctcaggctcccaaagtgctaggattacaggcgtgaaccaccacgcccagcctgttatttgtaaatgttgaatacatgttacattttcatcctaatgggctaaatttgcaccatttgccattcagaacaattctgtttctgaggtactctgttggtgctttagggccaactgggatctatttcagagaggaatggaataattgactgtaaatgtgatgaggaagaaataaacacttttaaaaaaaatgacacctaccatttattgaactcccatctacaaggcacttggctaagtacttcagaaaccactcacacttattaccctcagagtaggtatgttgaggcaacgagatcttagactcttgctcctatttaccccaactacactgttctgcttcccccagattattggtgtcagtgatggagacatttattaatcctgttagtttctgggagctagaaattgtgatttcttcttagtaatacaatcttgaataattttcaagctgatacccgtttagaagtatcagaagagaatttgtacatgaagcctgcacatacgtggggtgtaactcatgttcagttaggctaaaagttattgttgcgtgcctcttttcagaattttaggtacttgtgcttaaatttgattcagaactgttttggaaaagccttgagtatgtttgaaataccttccctcttgaaagtaatctcaagtttttaataagggttaatcatgttaaaaaaacaaaaatgtctattcaaccagacattggcatttcttgaccttttttcctgtcttacctggatcttgcaataaaggatgcctggtttaactttcttgaaaatcacattagggaaggctttgaatgaaattgatctggaacaataagtgatgatttggaaaaacaattgctatacttctatgtaccctgctgcagctctccccatgtctccacctctagaggtggggttcagggatttgcataactaaaaaatttatgaaagtgttgtcctacctttctcaggaacaccatttgtgaattattttcccaaaaacgaggtagaaattagaaatctagagaagtaactattagtacatgaggtcatattagtgttttcttgttgggtttttttttgtttatttggttttttatcttatggttttttatttatttgatttctttctttacgagacctcttgtggcggtggggggcggggaatgttcatttttttttaaacctatttgaccagcattgtttccttgaagaaaacctagatttcagatacagatgtttatgttttgatttatcttaattgctctggtttggtttttgggtttggtcagcactaacgtactaatgtggttaaaatgagtcctttgttttgggaggccaaggcgggtggatcacttcaggtcaggagttcaagaccagcctggtcaacatggcgaaactctgtctctactaaaaatacaaaaattagctgggactggtggcagaggcttgtaatcccagctactcaggaggctgaggcaagagaatcacttgaaccccggaggcaaaggttactgtgagccgagatcaggcctttgcactccagcctgggcaacaagtgaaactccgtctcaaaaacaaaacaaaacaaaaatgagtccttggtaactagaatattcggttcccagggttacagtatctagatagtaaataattcagggaagttagtggtaagagatttcttgatcatttctactgagaattttatttaacaagcattccttatgaaaaataatatctatgaaaaatttccttcatgaggaacgaaaactttcatttaatgaatgacaagggtatagttttaaaataaagggcaaaaatcaaaggttggtaaacgtgtgatctcagctctggaaaccccattatgcttatgtcaacggtgatgtctgagtgttgaggtttgggaaaggtgagtttccttgacttttcaaaaaattttagattttcgtatggtccaccatagacaaatgagtttaatcaaaagtcatagcttttttttttttttttttttttgcgacagagtctccgtctattgcccaggatggagtgcagtggcacaattttggctcactgcaacctccgcctcctgagttcaagctattctcctgcctcagcctcctgagtggctgggactacaggcatatgccaccacgcccagctagtttctgtatttttagtagagacagcatttcaccatattggccaggctggtctcgaactcctgacctagtgatccgcccacctcggcctcccaaagtgctgagattacaggtgtgagccaccatgcccagccaacttttatctttaagtaacttgtgatgtttcaattgcaaaatcctatgcctttgtgacttcaagtgacccctttcataatccataagtgtttaatgaatgtctaccatatacctagccttgacatggaaacatttttaatacaaatgtctatttttattttccttttgtttggtgtagagaaaaaatagccagttcacaatattttataaaatagttatgaagagaatgtcagtatactctacacatatcttgtttcatcttatcaagtaacactaccaacaatgtatagaatttcttcaaactgagttttatttggcttgtttggggatttttttttttttttttttttttttttggctaaaaagtaggtcctgaaaggaggacctccagaatgtgctttgtgtcattgtgtcgagtctttcttttgaaggtttaatatttaactatttatttaatataagcttttcttttgctgttagactgcttttcaggaagccttggacgctgcaggtgataaacttgtagtagttgacttctcagccacgtggtgtgggccttgcaaaatgatcaagcctttctttcatgtgagtattaaacaatgtctgctttgtaagagatttgtgttttttgagttggtggtcacagtggtaggaaagaaagacagttaaaggattttggtttcggtggggggatttctttggctggatctttggtctaaaagtagtagtataacaaataatttaggtttgatacatgtagcccattgaaaacaaattttagaagttaattttgtcttaaatagttctttttttccccacattgaaacatgggccttatttgaaatcccagcctcagaatttgatatgccaagctgttttatactaagaaaaatttgatttagagaaaatttatgtctcttagatctatgtctccaaagatctaaatttttggatctttaattagtctctacttttattaagtttccatttaagaagcttgggtatgttgattgccattacctagttctaaatctttttggatttttcattttaaattttccagtccctctctgaaaagtattccaacgtgatattccttgaagtagatgtggatgactgtcaggtatgtagctggaaatatgagatactgctgagcttttcacattggcctttttctctgaattgcacagtgcttttttccataaatatgtcaaataattctagaactgtaatcctatctaaaaagttctatctcagaagagcaggcaagttaggagcttaatcctagctatcgggagctgtatatcacatcctaaagtaaacaaaaataaatgagtgagacttctgaatcttatcggccacccacctttctaaaaccctacattctactttacactctgagatgtgcaataaatggagattgaatttagctatgatcattacatccataggcttgatggagtcaccaaattatgagaccgcttgtagggctctttgtgaacttgcagtagcatgagaacctgcatttgcaagcctattctagtcttggttgattttagtcaattagaaaccacaaatgttttaacaaataaacaccaaggtacctgagagaataatttggaagaaattccagggttggttgtatttaacaaatacttgttttgcactaggtatataccaggcactgttctgggtggtttttaagtatcagttcatttaatcctgagtgctgttatcatccccattttatagatgagaaaactgaaacacagaggttgttcatgaagtttcagtgagtatgtggctgaactaggatttaaaatgaagtggtctggcttcccagcccttgaccttaagcactacccatcggaggatgctctgtcttgtgggtgtagatcgggtgcttagcacatgaccacagacctaggagagcgggttgaggaggtatcacttcggggccctttacagatatgtgagcattttcacttagccctagtggagaaggaaaggcgatgggggaagggtgcagtgtggcaacagaggcgctggacctggcttccagtcctggctcactagcatctgcttaggccagtcactcctcttccttgagccttaagacctgccccatcaacctcccaaggttgcttattcattgagcaaacatggaatatccaataaagggtgaagggtcacttaaaacaggcatatggcagtgctctctaaacatgggagggcgcaacaaccccagattgtgtattcttagccagtttttgactctgtgccttgggcaacccctgccttggcttgtgctgtcttctccatctggcctgtcctttcctttcctacctgactaactccttgtcatgagcttcaccccttctccacttaccgccttgtgtgccctaagtacccagtgaatcttggcaattattataatgatctttatgtctgtcctttaccattagtctcagtagattcctaggatcagagaccctgtcttaattcacgttggttgccccttcacctagcacacctgccttgcatgtagtataggtgtggaatgaatgaatgatgaatgtgatatggttgttaagttactattctagatgtgtcccagagttgtttttttttttttaaaaagagtgtaattgcatttttgtgaaaaatccttatcccttgttttaatcaaacttagtcttattaaggtcaatttagctaggggaaaattgcacctggaatagagaaattctaactgccactgatcctatcagatagcaacttgattttttttttttttttttttttttttttttttttgagatggagttcactctgtcacctggggtggagtgcagtggcatgatctcggctcactgcaacctctgcctcccgggtttaagcaatcctctgtcctcagcctcctgggtagctgggattacaggcatgcaccaccatgcctggctaattttgtatttttacaaaattaaaaccccagtagagacggggtttcaccatgttggtcaggctggtctcgaactcctgacttcaggtgatccacccacctcggcctcccaaagtgctgggattatccaccacgcccggccttgatttttatttgaaagcaataataggtgccagatgccatgataagccctttgcatgcactatgtcatttaatcctcacgataactatacgagtattttttattagcaccctcattgaacaggtaatggcactgcagcacagaaggtaaagtcagtctcttgaggcagaccaatgcaccatactgtactgaggacaggtcttcttactgcctttaggaagtacagtcatgcatcacttaatgatgaggatactttctgagaaatgttaggcaattttgttgtgcacacatagagtgtacttacacaacctagatggcatagcctcctgtacacctaggctatgtggtaaagcctgttgctcctaggccacaaacaggtaaggcatgttactgtactgaatactgtaggcagttataacacaacagtaagtatttgtgtatctaaacatagaaaaggtatggtgaaaacatgatatgaaagattaaaaaatggtatgcctttataaggtacttgccataaacgggacttgcaggactgagaggtgctctgggcgagtcagtgagtgggtggtgagtgaatgtgaaggcctagaacctgtagacgttataaacactgtatgcttacaaattttatttttaaaatttcttttttcagcaataaatttattgtaacttttttactttatagtttttttatttttttaactctttaactcttgtaataacacttagcttaaaacacgaatgcattgtacagctctacaaaaatattttctttatatccttagtctataagctttttttaaaaagactttttaaactttttgttacaaactaagattcaaacacatacattagcctagaccaacacagggtcaggatcatcagtatcactgtctcccatccccacatcttgtctcacagaaaggtcttcagggacagtaacatgcatggacctttcatctcctatgataacagtgccttctcctggaatgcttcctgaagggcctgcctgagcctgttgtatagtaactgtctttttaaaaaaataagtaggagtacactctaaattaataatgaaattaaagtaaatacaaaaaccagtaacgtgggtgtttattatcaagtagtatatactgtccataattgtagtgatatgcttttttaagtgaaagcaagtttattaagaaagtaaaggaacaaaagaatggctattccgcaggtaaagcagtctgtagtggtatactttgtatgtaattgcagcgcagatttgtttgcaccagctaatgcgatgggctatgacattaacccatcactaggtgagaggaatttttcaacttcattataatcttatgggaccaccacatatatgcaatctgttgtcgatctaaatgttatatggtgcattactataggtgtgcaaagcactcgaggacttccgtatgacagagctcctccttcatgtctgcttggtgcaccctgatcaccctgaatgtatcttttttttttttttttttgagacagagtctcactctgtcacccaggctggagtgcagtggtaccatctctgctcactgcaacctccacctcccgggttcaagcgattcttctgcctcagcctcctgagtagctgggactacaggcagccgctaccacaccaggctaaatttcaactttttagtagagacagcattttgccatgttggccaggctggtctcgaactcctcacctcaagtgatccacccgtcttggcctcccaaagtgctgggattagaggcatgagctaccgtgcctggcctgaaagtgtcttttaaaaccttgaagtgaccctctgacaaactgaggaactttaactttgcctccatagattgatagaaaagtatgagtagtagcccttttgaaaatgatagaccaaccttatttctctgacagccaacagggttatgatacttattttataaatggtaacctccctctgacccttacttggagtgagttttcaatagtatgcattcaataaacattcaccatttttattccagccattactgtccttgtgcctcttactggaacctgtactttcatgctcagcaggtgtccagcattaaaagaaaaagtaaagattacctagaaagaactcctcaacagtagtgccacccaccatcctagaggtcgtcatagtgtttgtagctggcctttcttccccttgagaattctccgttggtttccgtgatttggttatcaacagtcctgcctgctcgcttgctgtcctgtgtagcttttgctgcttaggtgctgagtggttctatatttctttcccagtcctcttttgagtgcctggctgacattttcaatctctattgggctccaaaccaaaccagtttcgtggtattgtcctccaaaccttgccctcttatagcatgaacaatgtgttgagcatggggtattataagagttctcatttagcattccacagttgaggaatgtgtgttacttcaattacctttgagctgtagaaaaatctttagctgtggtaacagccacttctaggagaggagaaaatacggatcaactagcccaatttgcgatgttaggaatttgtcgattttcttagtaggatggctttcaaaggttagagcatcagagtcacctgaagcccgactttaactgtaatggtttaagatggggttgatggggaaacttgtagtacccctcaggtaattctgatactgcagcaaggtttgagaattcacaaagtctttttatttttcctcccgagatagtctcattctgtcgcccaggctggaatacagtggcatgatctcagctcactgcaacctccgcctcccaggttcaagcaattgtcctgtctcaacctcctgagtagctgggatgacagatgtgtgccaccacacctggctaatttttgtatttttagtagagatggggtttctccgtgttagccaggctagtctcgaactcctgacctcaggtgacccaccggccttggcctcacaaatcagtttttaattaaaaataagcaggaggctgagtgtggtggctcacacctgtaatcctagcactttgggagcccgaggaaggtggatcacttgagctcatgagtttgagaccagcctgggcaacatggagagaccttgtccctataaaaaaaaaaaaaaaaaaatatatatatatatatatatatatatatatatatatagtgtgtgtgtgtgtgtatatatatacacacacacacacaaaattagccaggtgtggtggcgtgtgcctgtagtcccagctactggggaggctgaggagggaggatggcttgagtctgggaagtggagattgcagtgagctgagactatgcccctgcattccatccagcctgggtgacacagccagaccctgtctcaaaaataataataatcagtaaacccagtgtggggttattcctttagattactattattttgttcttgaacaattgatttttatttttttagactttttagcctttatataatcattctgtgtactctgccttcataataaaactggaaaaattatgagcaagaaataagaggtactagttctgaggaatagttaagattatcatactgagtccaattgtagcagaattttttgttgcttctttgtatgatacttaaaatagttgaaaatttgattggattaaagagcatattggatcgctggagtatctgatgctagtaacattctgaacattctgcctgttaatgtgcccgtcaaaggaagtaaatattaataaaacttcttcattgagaatataaccggtttggcttttgtactgccattatattcattatattaattttcatatgctgaaaaatgtcctcatgcggaaatgtggggtacatgacagggaaaagtttctggttttggattacttctgtcaaagctcagtactcgcagtcttgtatttaatcctctccctttgctactttccctaccaggatgttgcttcagagtgtgaagtcaaatgcatgccaacattccagttttttaagaagggacaaaaggtacgtacatctgacctttaaaactctaacttctaactgggcaataggaaacccagtataagtgaataaatcactggagtgatgttccctttaaagattgaggcatatcaccaagttctgcttttaagaatttttaaatatgccaaaattcattggcttaagtacataatgtgacagctaactgaaaatcaatctttcctagaactagtcctatttatatcataaagcacatagaatttcttagacttgggcagttcatttgttgttaagtattgtgtaaaagaaaatttgtacttgagccttttgacttttctcttgatattttttctttgtttataacttaaatgaactgtatgttattcagggaagtttattttaaataagattatacttctttttccctccacccctattcttccttcattctatgctgaatacatatttatacatatgtatatatatacatatgtatatgtatatatataaatacatatttatacatattttatgtataaaacagtgctacagtgctacgtctaatgtcaattcaatattctcttaacaggtgggtgaattttctggagccaataaggaaaagcttgaagccaccattaatgaattagtctaatcatgttttctgaaaacataaccagccattggctatttaaaacttgtaatttttttaatttacaaaaatataaaatatgaagacataaacccagttgccatctgcgtgacaataaaacattaatgctaacactttttaaaaccgtctcgtgtctgaatagctttcaaaataaatgtgaaatggtcatttaatgtattttcctatattctcaatcactttttagtaaccttgtaggccactgattattttaagattttaaaaattattattgctaccttaatgtattgctacaaaaatctcttgttgggggcaatgcaggtaataaagtagtatgttgttatttgttatcttttgacagagaaaatagcattctctgttttagcaggtgaatcctctatgctctccaaaagatcagcatgaccaaaattgatgtccactcatgaaggacttgttcgttttgtttgtttgttttgccacgaggatcggatcttgattctcctcgaagtatctgagaaagtctagttgtataggccagacataggttctgtcttgagtggtaaaagttgtgggaaattattacttatatcattcaaagaacattgtttcttgtgttctaagcacagtaaggggttgggggtttgaagaaactttttgagtttacataataatgaagagcagaattatcatatgccaggtatcatctttctattaattcacttaatctttgtaacaatcctattagatatagacactataatccccatttacctgtgaggaaacagacaaagggtggtaatttccccagcatcacaactagtcattggaggagctgggatttgaaccaaagaagtctggtaccagaatatgtgcctttaactactacattgccctcagtgcaacaatctgagtaagcaggaaaatgatgggcccttagttgagtttctttcctcatgtgaaattaggatgccaaacttagatgatctctcaaaatactaatggaatgcctgttatgtgccaggcatcatgctaggcttgggataaagctgtgactaagacaccctcatcctcatacagcttacattctagaagcagagacaaactggtgaataatagactggtatattctacaaggcaaaaccaaaaactgggcgcagtaatgagaagaataggggaacccgctgtgcatcaggtggttggacctgggttctttgaggtgactgtaaacagagctgttgg

SEQ ID NO: 5 - DNA, 16 bp, Artificial Sequence
  Description of Artificial Sequence Synthetic oligonucleotide
tccaaagcaccaaaca

SEQ ID NO: 6 - DNA, 16 bp, Artificial Sequence
  Description of Artificial Sequence Synthetic oligonucleotide
aaggaccgatggaaat

SEQ ID NO: 7 - DNA, 16 bp, Artificial Sequence
  Description of Artificial Sequence Synthetic oligonucleotide
ttttcagagagggaat

SEQ ID NO: 8 - DNA, 16 bp, Artificial Sequence
  Description of Artificial Sequence Synthetic oligonucleotide
caaggaatatcacgtt

SEQ ID NO: 9 - DNA, 16 bp, Artificial Sequence
  Description of Artificial Sequence Synthetic oligonucleotide
tggaatgttggcgtgc

SEQ ID NO: 10 - DNA, 16 bp, Artificial Sequence
  Description of Artificial Sequence Synthetic oligonucleotide
tccttattggctccag

SEQ ID NO: 11 - DNA, 16 bp, Artificial Sequence
  Description of Artificial Sequence Synthetic oligonucleotide
gcttcaccatcttggc

SEQ ID NO: 12 - DNA, 16 bp, Artificial Sequence
  Description of Artificial Sequence Synthetic oligonucleotide
gacgagcggctgtaag

SEQ ID NO: 13 - DNA, 16 bp, Artificial Sequence
  Description of Artificial Sequence Synthetic oligonucleotide
caaggcccacaccacg

SEQ ID NO: 14 - DNA, 16 bp, Artificial Sequence
  Description of Artificial Sequence Synthetic oligonucleotide
ctactacaagtttatc

SEQ ID NO: 15 - DNA, 16 bp, Artificial Sequence
  Description of Artificial Sequence Synthetic oligonucleotide
cagtcttgctctcgat

SEQ ID NO: 16 - DNA, 16 bp, Artificial Sequence
  Description of Artificial Sequence Synthetic oligonucleotide
aagcaacatcctgaca

SEQ ID NO: 17 - DNA, 16 bp, Artificial Sequence
  Description of Artificial Sequence Synthetic oligonucleotide
ctcgtccttctcctcc

SEQ ID NO: 18 - DNA, 16 bp, Artificial Sequence
  Description of Artificial Sequence Synthetic oligonucleotide
catcttcctccagtcg

SEQ ID NO: 19 - DNA, 16 bp, Artificial Sequence
  Description of Artificial Sequence Synthetic oligonucleotide
acagagcttcaagact

SEQ ID NO: 20 - DNA, 16 bp, Artificial Sequence
  Description of Artificial Sequence Synthetic oligonucleotide
ggatccaaagcaccaa

SEQ ID NO: 21 - DNA, 16 bp, Artificial Sequence
  Description of Artificial Sequence Synthetic oligonucleotide
aaggaccgatggaaat

SEQ ID NO: 22 - DNA, 16 bp, Artificial Sequence
  Description of Artificial Sequence Synthetic oligonucleotide
tctgacgagcggctgt

SEQ ID NO: 23 - DNA, 16 bp, Artificial Sequence
  Description of Artificial Sequence Synthetic oligonucleotide
atcttggctgctggag

SEQ ID NO: 24 - DNA, 16 bp, Artificial Sequence
  Description of Artificial Sequence Synthetic oligonucleotide
ctcgatctgcttcacc

SEQ ID NO: 25 - DNA, 16 bp, Artificial Sequence
  Description of Artificial Sequence Synthetic oligonucleotide
gaaaagcagtcttgct

SEQ ID NO: 26 - DNA, 16 bp, Artificial Sequence
  Description of Artificial Sequence Synthetic oligonucleotide
gcgtccaaggcttcct

SEQ ID NO: 27 - DNA, 16 bp, Artificial Sequence
  Description of Artificial Sequence Synthetic oligonucleotide
aagtttatcacctgca

SEQ ID NO: 28 - DNA, 16 bp, Artificial Sequence
  Description of Artificial Sequence Synthetic oligonucleotide
agaagtcaactactac

SEQ ID NO: 29 - DNA, 16 bp, Artificial Sequence
  Description of Artificial Sequence Synthetic oligonucleotide
ccacaccacgtggctg

SEQ ID NO: 30 - DNA, 16 bp, Artificial Sequence
  Description of Artificial Sequence Synthetic oligonucleotide
gatcattttgcaaggc

SEQ ID NO: 31 - DNA, 16 bp, Artificial Sequence
  Description of Artificial Sequence Synthetic oligonucleotide
aatgaaagaaaggctt

SEQ ID NO: 32 - DNA, 16 bp, Artificial Sequence
  Description of Artificial Sequence Synthetic oligonucleotide
tacttttcagagaggg

SEQ ID NO: 33 - DNA, 16 bp, Artificial Sequence
  Description of Artificial Sequence Synthetic oligonucleotide
gaatatcacgttggaa

SEQ ID NO: 34 - DNA, 16 bp, Artificial Sequence
  Description of Artificial Sequence Synthetic oligonucleotide
ccacatctacttcaag

SEQ ID NO: 35 - DNA, 16 bp, Artificial Sequence
  Description of Artificial Sequence Synthetic oligonucleotide
acatcctgacagtcat

SEQ ID NO: 36 - DNA, 16 bp, Artificial Sequence
  Description of Artificial Sequence Synthetic oligonucleotide
ttcacactctgaagca

SEQ ID NO: 37 - DNA, 16 bp, Artificial Sequence
  Description of Artificial Sequence Synthetic oligonucleotide
ttggcatgcatttgac

SEQ ID NO: 38 - DNA, 16 bp, Artificial Sequence
  Description of Artificial Sequence Synthetic oligonucleotide
ttaaaaaactggaatg

SEQ ID NO: 39 - DNA, 16 bp, Artificial Sequence
  Description of Artificial Sequence Synthetic oligonucleotide
caccttttgtcccttc

SEQ ID NO: 40 - DNA, 16 bp, Artificial Sequence
  Description of Artificial Sequence Synthetic oligonucleotide
ctccagaaaattcacc

SEQ ID NO: 41 - DNA, 16 bp, Artificial Sequence
  Description of Artificial Sequence Synthetic oligonucleotide
agcttttccttattgg

SEQ ID NO: 42 - DNA, 16 bp, Artificial Sequence
  Description of Artificial Sequence Synthetic oligonucleotide
attaatggtggcttca

SEQ ID NO: 43 - DNA, 16 bp, Artificial Sequence
  Description of Artificial Sequence Synthetic oligonucleotide
atgattagactaattc

SEQ ID NO: 44 - DNA, 16 bp, Artificial Sequence
  Description of Artificial Sequence Synthetic oligonucleotide
ttatattttcagaaac

SEQ ID NO: 45 - DNA, 16 bp, Artificial Sequence
  Description of Artificial Sequence Synthetic oligonucleotide
atagctcaatggctgg

SEQ ID NO: 46 - DNA, 16 bp, Artificial Sequence
  Description of Artificial Sequence Synthetic oligonucleotide
aaattacaagttttaa

SEQ ID NO: 47 - DNA, 16 bp, Artificial Sequence
  Description of Artificial Sequence Synthetic oligonucleotide
tttttgtaaattaaaa

SEQ ID NO: 48 - DNA, 16 bp, Artificial Sequence
  Description of Artificial Sequence Synthetic oligonucleotide
gtcttcatattttata

SEQ ID NO: 49 - DNA, 16 bp, Artificial Sequence
  Description of Artificial Sequence Synthetic oligonucleotide
tggcaactgggtttat

SEQ ID NO: 50 - DNA, 16 bp, Artificial Sequence
  Description of Artificial Sequence Synthetic oligonucleotide
tttattgtcacgcaga

SEQ ID NO: 51 - DNA, 16 bp, Artificial Sequence
  Description of Artificial Sequence Synthetic oligonucleotide
gtgttagcattaatgt

SEQ ID NO: 52 - DNA, 16 bp, Artificial Sequence
  Description of Artificial Sequence Synthetic oligonucleotide
gagacggttttaaaaa

SEQ ID NO: 53 - DNA, 16 bp, Artificial Sequence
  Description of Artificial Sequence Synthetic oligonucleotide
aaagctattcagacat

SEQ ID NO: 54 - DNA, 16 bp, Artificial Sequence
  Description of Artificial Sequence Synthetic oligonucleotide
tttcacatttattttg

SEQ ID NO: 55 - DNA, 16 bp, Artificial Sequence
  Description of Artificial Sequence Synthetic oligonucleotide
cgctgcttgctctctc

SEQ ID NO: 56 - DNA, 16 bp, Artificial Sequence
  Description of Artificial Sequence Synthetic oligonucleotide
cctttataaactggca

SEQ ID NO: 57 - DNA, 16 bp, Artificial Sequence
  Description of Artificial Sequence Synthetic oligonucleotide
aactggcacgcccggc

SEQ ID NO: 58 - DNA, 23 bp, Artificial Sequence
  Description of Artificial Sequence Synthetic oligonucleotide primer
aagcctttctttcattccctctc

SEQ ID NO: 59 - DNA, 25 bp, Artificial Sequence
  Description of Artificial Sequence Synthetic oligonucleotide primer
cttcttaaaaaactggaatgttggc

SEQ ID NO: 60 - DNA, 29 bp, Artificial Sequence
  Description of Artificial Sequence Synthetic oligonucleotide probe
gatgtggatgactgtcaggatgttgcttc

SEQ ID NO: 61 - DNA, 21 bp, Artificial Sequence
  Description of Artificial Sequence Synthetic oligonucleotide primer
aaggctgtgggcaaggtcatc

SEQ ID NO: 62 - DNA, 23 bp, Artificial Sequence
  Description of Artificial Sequence Synthetic oligonucleotide primer
gtcagatccacgacggacacatt

SEQ ID NO: 63 - DNA, 34 bp, Artificial Sequence
  Description of Artificial Sequence Synthetic oligonucleotide probe
gaagctcactggcatggcatggccttccgtgttc

SEQ ID NO: 64 - DNA, 20 bp, Artificial Sequence
  Description of Artificial Sequence Synthetic oligonucleotide primer
ggatccatttccatcggtcc

SEQ ID NO: 65 - DNA, 23 bp, Artificial Sequence
  Description of Artificial Sequence Synthetic oligonucleotide primer
gcagatggcaactggttatgtct

SEQ ID NO: 66 - DNA, 18 bp, Artificial Sequence
  Description of Artificial Sequence Synthetic oligonucleotide primer
aacggatttggtcgtatt

SEQ ID NO: 67 - DNA, 18 bp, Artificial Sequence
  Description of Artificial Sequence Synthetic oligonucleotide primer
taagcagttggtggtgca

SEQ ID NO: 68 - DNA, 16 bp, Artificial Sequence
  Description of Artificial Sequence Synthetic oligonucleotide
tccaaagcaccaaaca

SEQ ID NO: 69 - DNA, 16 bp, Artificial Sequence
  Description of Artificial Sequence Synthetic oligonucleotide
tccaaagcaccaaaca

SEQ ID NO: 70 - DNA, 16 bp, Artificial Sequence
  Description of Artificial Sequence Synthetic oligonucleotide
tccaaagcaccaaaca

SEQ ID NO: 71 - DNA, 16 bp, Artificial Sequence
  Description of Artificial Sequence Synthetic oligonucleotide
aaggaccgatggaaat

SEQ ID NO: 72 - DNA, 16 bp, Artificial Sequence
  Description of Artificial Sequence Synthetic oligonucleotide
aaggaccgatggaaat

SEQ ID NO: 73 - DNA, 16 bp, Artificial Sequence
  Description of Artificial Sequence Synthetic oligonucleotide
aaggaccgatggaaat

SEQ ID NO: 74 - DNA, 16 bp, Artificial Sequence
  Description of Artificial Sequence Synthetic oligonucleotide
ttttcagagagggaat

SEQ ID NO: 75 - DNA, 16 bp, Artificial Sequence
  Description of Artificial Sequence Synthetic oligonucleotide
ttttcagagagggaat

SEQ ID NO: 76 - DNA, 16 bp, Artificial Sequence
  Description of Artificial Sequence Synthetic oligonucleotide
ttttcagagagggaat

SEQ ID NO: 77 - DNA, 16 bp, Artificial Sequence
  Description of Artificial Sequence Synthetic oligonucleotide
caaggaatatcacgtt

SEQ ID NO: 78 - DNA, 16 bp, Artificial Sequence
  Description of Artificial Sequence Synthetic oligonucleotide
caaggaatatcacgtt

SEQ ID NO: 79 - DNA, 16 bp, Artificial Sequence
  Description of Artificial Sequence Synthetic oligonucleotide
caaggaatatcacgtt

SEQ ID NO: 80 - DNA, 16 bp, Artificial Sequence
  Description of Artificial Sequence Synthetic oligonucleotide
tggaatgttggcgtgc

SEQ ID NO: 81 - DNA, 16 bp, Artificial Sequence
  Description of Artificial Sequence Synthetic oligonucleotide
tggaatgttggcgtgc

SEQ ID NO: 82 - DNA, 16 bp, Artificial Sequence
  Description of Artificial Sequence Synthetic oligonucleotide
tggaatgttggcgtgc

SEQ ID NO: 83 - DNA, 16 bp, Artificial Sequence
  Description of Artificial Sequence Synthetic oligonucleotide
tccttattggctccag

SEQ ID NO: 84 - DNA, 16 bp, Artificial Sequence
  Description of Artificial Sequence Synthetic oligonucleotide
tccttattggctccag

SEQ ID NO: 85 - DNA, 16 bp, Artificial Sequence
  Description of Artificial Sequence Synthetic oligonucleotide
tccttattggctccag

SEQ ID NO: 86 - DNA, 16 bp, Artificial Sequence
  Description of Artificial Sequence Synthetic oligonucleotide
gcttcaccatcttggc

SEQ ID NO: 87 - DNA, 16 bp, Artificial Sequence
  Description of Artificial Sequence Synthetic oligonucleotide
gcttcaccatcttggc

SEQ ID NO: 88 - DNA, 16 bp, Artificial Sequence
  Description of Artificial Sequence Synthetic oligonucleotide
gcttcaccatcttggc

SEQ ID NO: 89 - DNA, 16 bp, Artificial Sequence
  Description of Artificial Sequence Synthetic oligonucleotide
gacgagcggctgtaag

SEQ ID NO: 90 - DNA, 16 bp, Artificial Sequence
  Description of Artificial Sequence Synthetic oligonucleotide
gacgagcggctgtaag

SEQ ID NO: 91 - DNA, 16 bp, Artificial Sequence
  Description of Artificial Sequence Synthetic oligonucleotide
gacgagcggctgtaag

SEQ ID NO: 92 - DNA, 16 bp, Artificial Sequence
  Description of Artificial Sequence Synthetic oligonucleotide
caaggcccacaccacg

SEQ ID NO: 93 - DNA, 16 bp, Artificial Sequence
  Description of Artificial Sequence Synthetic oligonucleotide
caaggcccacaccacg

SEQ ID NO: 94 - DNA, 16 bp, Artificial Sequence
  Description of Artificial Sequence Synthetic oligonucleotide
caaggcccacaccacg

SEQ ID NO: 95 - DNA, 16 bp, Artificial Sequence
  Description of Artificial Sequence Synthetic oligonucleotide
ctactacaagtttatc

SEQ ID NO: 96 - DNA, 16 bp, Artificial Sequence
  Description of Artificial Sequence Synthetic oligonucleotide
ctactacaagtttatc

SEQ ID NO: 97 - DNA, 16 bp, Artificial Sequence
  Description of Artificial Sequence Synthetic oligonucleotide
ctactacaagtttatc

SEQ ID NO: 98 - DNA, 16 bp, Artificial Sequence
  Description of Artificial Sequence Synthetic oligonucleotide
cagtcttgctctcgat

SEQ ID NO: 99 - DNA, 16 bp, Artificial Sequence
  Description of Artificial Sequence Synthetic oligonucleotide
cagtcttgctctcgat

SEQ ID NO: 100 - DNA, 16 bp, Artificial Sequence
  Description of Artificial Sequence Synthetic oligonucleotide
cagtcttgctctcgat

SEQ ID NO: 101 - DNA, 16 bp, Artificial Sequence
  Description of Artificial Sequence Synthetic oligonucleotide
aagcaacatcctgaca

SEQ ID NO: 102 - DNA, 16 bp, Artificial Sequence
  Description of Artificial Sequence Synthetic oligonucleotide
aagcaacatcctgaca

SEQ ID NO: 103 - DNA, 16 bp, Artificial Sequence
  Description of Artificial Sequence Synthetic oligonucleotide
aagcaacatcctgaca

SEQ ID NO: 104 - DNA, 16 bp, Artificial Sequence
  Description of Artificial Sequence Synthetic oligonucleotide
ctcgtccttctcctcc

SEQ ID NO: 105 - DNA, 16 bp, Artificial Sequence
  Description of Artificial Sequence Synthetic oligonucleotide
ctcgtccttctcctcc

SEQ ID NO: 106 - DNA, 16 bp, Artificial Sequence
  Description of Artificial Sequence Synthetic oligonucleotide
ctcgtccttctcctcc

SEQ ID NO: 107 - DNA, 16 bp, Artificial Sequence
  Description of Artificial Sequence Synthetic oligonucleotide
catcttcctccagtcg

SEQ ID NO: 108 - DNA, 16 bp, Artificial Sequence
  Description of Artificial Sequence Synthetic oligonucleotide
catcttcctccagtcg

SEQ ID NO: 109 - DNA, 16 bp, Artificial Sequence
  Description of Artificial Sequence Synthetic oligonucleotide
catcttcctccagtcg

SEQ ID NO: 110 - DNA, 16 bp, Artificial Sequence
  Description of Artificial Sequence Synthetic oligonucleotide
acagagcttcaagact

SEQ ID NO: 111 - DNA, 16 bp, Artificial Sequence
  Description of Artificial Sequence Synthetic oligonucleotide
ggatccaaagcaccaa

SEQ ID NO: 112 - DNA, 16 bp, Artificial Sequence
  Description of Artificial Sequence Synthetic oligonucleotide
aaggaccgatggaaat

SEQ ID NO: 113 - DNA, 16 bp, Artificial Sequence
  Description of Artificial Sequence Synthetic oligonucleotide
tctgacgagcggctgt

SEQ ID NO: 114 - DNA, 16 bp, Artificial Sequence
  Description of Artificial Sequence Synthetic oligonucleotide
atcttggctgctggag

SEQ ID NO: 115 - DNA, 16 bp, Artificial Sequence
  Description of Artificial Sequence Synthetic oligonucleotide
ctcgatctgcttcacc

SEQ ID NO: 116 - DNA, 16 bp, Artificial Sequence
  Description of Artificial Sequence Synthetic oligonucleotide
gaaaagcagtcttgct

SEQ ID NO: 117 - DNA, 16 bp, Artificial Sequence
  Description of Artificial Sequence Synthetic oligonucleotide
gcgtccaaggcttcct

SEQ ID NO: 118 - DNA, 16 bp, Artificial Sequence
  Description of Artificial Sequence Synthetic oligonucleotide
aagtttatcacctgca

SEQ ID NO: 119 - DNA, 16 bp, Artificial Sequence
  Description of Artificial Sequence Synthetic oligonucleotide
agaagtcaactactac

SEQ ID NO: 120 - DNA, 16 bp, Artificial Sequence
  Description of Artificial Sequence Synthetic oligonucleotide
ccacaccacgtggctg

SEQ ID NO: 121 - DNA, 16 bp, Artificial Sequence
  Description of Artificial Sequence Synthetic oligonucleotide
gatcattttgcaaggc

SEQ ID NO: 122 - DNA, 16 bp, Artificial Sequence
  Description of Artificial Sequence Synthetic oligonucleotide
aatgaaagaaaggctt

SEQ ID NO: 123 - DNA, 16 bp, Artificial Sequence
  Description of Artificial Sequence Synthetic oligonucleotide
tacttttcagagaggg

SEQ ID NO: 124 - DNA, 16 bp, Artificial Sequence
  Description of Artificial Sequence Synthetic oligonucleotide
gaatatcacgttggaa

SEQ ID NO: 125 - DNA, 16 bp, Artificial Sequence
  Description of Artificial Sequence Synthetic oligonucleotide
ccacatctacttcaag

SEQ ID NO: 126 - DNA, 16 bp, Artificial Sequence
  Description of Artificial Sequence Synthetic oligonucleotide
acatcctgacagtcat

SEQ ID NO: 127 - DNA, 16 bp, Artificial Sequence
  Description of Artificial Sequence Synthetic oligonucleotide
ttcacactctgaagca

SEQ ID NO: 128 - DNA, 16 bp, Artificial Sequence
  Description of Artificial Sequence Synthetic oligonucleotide
ttggcatgcatttgac

SEQ ID NO: 129 - DNA, 16 bp, Artificial Sequence
  Description of Artificial Sequence Synthetic oligonucleotide
ttaaaaaactggaatg

SEQ ID NO: 130 - DNA, 16 bp, Artificial Sequence
  Description of Artificial Sequence Synthetic oligonucleotide
caccttttgtcccttc

SEQ ID NO: 131 - DNA, 16 bp, Artificial Sequence
  Description of Artificial Sequence Synthetic oligonucleotide
ctccagaaaattcacc

SEQ ID NO: 132 - DNA, 16 bp, Artificial Sequence
  Description of Artificial Sequence Synthetic oligonucleotide
agcttttccttattgg

SEQ ID NO: 133 - DNA, 16 bp, Artificial Sequence
  Description of Artificial Sequence Synthetic oligonucleotide
attaatggtggcttca

SEQ ID NO: 134 - DNA, 16 bp, Artificial Sequence
  Description of Artificial Sequence Synthetic oligonucleotide
atgattagactaattc

SEQ ID NO: 135 - DNA, 16 bp, Artificial Sequence
  Description of Artificial Sequence Synthetic oligonucleotide
ttatattttcagaaac

SEQ ID NO: 136 - DNA, 16 bp, Artificial Sequence
  Description of Artificial Sequence Synthetic oligonucleotide
atagctcaatggctgg

SEQ ID NO: 137 - DNA, 16 bp, Artificial Sequence
  Description of Artificial Sequence Synthetic oligonucleotide
aaattacaagttttaa

SEQ ID NO: 138 - DNA, 16 bp, Artificial Sequence
  Description of Artificial Sequence Synthetic oligonucleotide
tttttgtaaattaaaa

SEQ ID NO: 139 - DNA, 16 bp, Artificial Sequence
  Description of Artificial Sequence Synthetic oligonucleotide
gtcttcatattttata

SEQ ID NO: 140 - DNA, 16 bp, Artificial Sequence
  Description of Artificial Sequence Synthetic oligonucleotide
tggcaactgggtttat

SEQ ID NO: 141 - DNA, 16 bp, Artificial Sequence
  Description of Artificial Sequence Synthetic oligonucleotide
tttattgtcacgcaga

SEQ ID NO: 142 - DNA, 16 bp, Artificial Sequence
  Description of Artificial Sequence Synthetic oligonucleotide
gtgttagcattaatgt

SEQ ID NO: 143 - DNA, 16 bp, Artificial Sequence
  Description of Artificial Sequence Synthetic oligonucleotide
gagacggttttaaaaa

SEQ ID NO: 144 - DNA, 16 bp, Artificial Sequence
  Description of Artificial Sequence Synthetic oligonucleotide
aaagctattcagacat

SEQ ID NO: 145 - DNA, 16 bp, Artificial Sequence
  Description of Artificial Sequence Synthetic oligonucleotide
tttcacatttattttg

SEQ ID NO: 146 - DNA, 16 bp, Artificial Sequence
  Description of Artificial Sequence Synthetic oligonucleotide
cgctgcttgctctctc

SEQ ID NO: 147 - DNA, 16 bp, Artificial Sequence
  Description of Artificial Sequence Synthetic oligonucleotide
cctttataaactggca

SEQ ID NO: 148 - DNA, 16 bp, Artificial Sequence
  Description of Artificial Sequence Synthetic oligonucleotide
aactggcacgcccggc

SEQ ID NO: 149 - protein, 4 aa, Artificial Sequence
  Description of Artificial Sequence Synthetic peptide
CGPC

SEQ ID NO: 150 - DNA, 18 bp, Artificial Sequence
  Description of Artificial Sequence Synthetic poly-T oligonucleotide
tttttttttttttttttt